(19) United States
(12) Patent Application Publication
(10) Pub. No.: US 2019/0111423 A1
(43) Pub. Date: Apr. 18, 2019

(54) DEVICES AND METHODS FOR AUTONOMOUS MEASUREMENTS

(71) Applicant: CALIFORNIA INSTITUTE OF TECHNOLOGY, Pasadena, CA (US)

(72) Inventors: Rustem F. ISMAGILOV, Altadena, CA (US); Stefano BEGOLO, Pasadena, CA (US); Jesus Rodriguez MANZANO, Pasadena, CA (US)

(21) Appl. No.: 15/539,551

(22) PCT Filed: Dec. 23, 2015
(86) PCT No.: PCT/US15/00243

§ 371 Date: Jun. 23, 2017

Related U.S. Application Data
(60) Provisional application No. 62/096,131, filed on Dec. 23, 2014.
(60) Provisional application No. 62/135,041, filed on Mar. 18, 2015.

(51) Int. Cl. B01L 3/00; C12Q 1/686; B01L 7/00

(52) U.S. Cl. CPC B01L 3/50273; C12Q 1/686; B01L 3/502723; B01L 3/502746; B01L 3/502753; B01L 2400/0487; B01L 2200/025; B01L 2200/0647; B01L 2200/0689; B01L 2200/16; B01L 2300/048; B01L 7/52

(57) ABSTRACT

Disclosed herein are devices and methods for storing, processing, preparing and/or analyzing samples. The inventions herein also relate to strategies and methods for automating device operations and for combining multiple devices in an integrated platform.

### CROSS-REFERENCE

This application claims the benefit of U.S. Provisional Application No. 62/096,131, filed Dec. 23, 2014, and U.S. Provisional Application No. 62/135,041, filed Mar. 18, 2015, these applications incorporated herein by reference in their entirety.

### STATEMENT AS TO FEDERALLY SPONSORED RESEARCH

This invention was made with support of the United States government under contract number HR0011-11-2-0006 by the Defense Advanced Research Projects Agency. The government has certain rights in the invention.

### BACKGROUND OF THE INVENTION

Modern biological techniques, including nucleic acid analysis, offer powerful tools for the analysis of samples. Samples from subjects and environmental sources can be analyzed for the presence of various compounds and organisms. Patients can be diagnosed for diseases, including infectious diseases and genetic diseases.

However, many analysis techniques require centralized laboratory facilities, trained technicians, sample preparation, refrigeration, and other resources. Such requirements can limit the utility of these techniques in point-of-care settings, limited resource settings, and other environments with difficult or no access to necessary resources.

What is needed, therefore, is a simple, inexpensive, robust method for nucleic acid based diagnostics at the point of care with immediate availability of results while the health care providers are still with the patient. In addition, a method and device are needed that can provide accuracy results and methodological adherence to proper analytic techniques, as well as quality control measures, particularly those that will permit waiver under the Clinical Laboratory Improvement Amendments (CLIA) program.

### SUMMARY OF THE INVENTION

Disclosed herein are devices and methods for storing, processing, preparing and/or analyzing samples. The inventions herein also relate to strategies and methods for automating device operations and for combining multiple devices in an integrated platform.

In some embodiments, disclosed herein are devices and systems for multi-step fluid management automated by a driving module configured to provide torque to rotate a shaft in the device. In some embodiments, the driving module provides a constant torque. In some embodiments, the driving module comprises a constant torque spring. In some embodiments, the shaft is used to control pressure in a cavity and the speed of the movement of parts in the device. In some embodiments the pressure in the cavity can be reduced by creating reversible connections between the cavity and the external atmosphere. In some embodiments, when the connection is present, the air in the cavity is free to escape and the pressure is reduced. This situation can be referred to as “venting,” since the extra pressure in the cavity is released through a venting connection.

In some embodiments, the shaft is a central threaded shaft. In some embodiments, the central threaded shaft is rotated by the driving module. In some embodiments, the central threaded shaft is attached to a rotor layer and a pressure cap. Rotation of the central shaft rotates the rotor layer and moves the pressure cap down or up to increase or decrease pressure in a pressure chamber. An increased pressure slows the rotation of the rotor layer as continued rotation also further increases pressure. In some embodiments, the rotor layer and the device has via holes. In some embodiments, rotation of the rotor layer can align the via holes, thereby creating a fluidic channel to transfer fluid. In some embodiments, the aligned via holes also act as a vent, releasing pressure from the pressure chamber, and increasing the rotational velocity of the rotor layer and central threaded shaft under a constant torque.

In some embodiments of the device, the pressure build-up and venting strategy can be used to pre-program the speed of rotation, such as for example by producing venting holes that align with the moving part in different positions: speed can be reduced by building up pressure, and then increased by inducing venting of the extra pressure in the cavity. In some embodiments, a multistep processes can be pre-programmed by the device design, and actuated by a single spring.

In some embodiments, the device is configured to generate one or more disrupting fluid paths and for generating and releasing pressure, while affecting the rotational velocity of a layer in the device using a single mechanical movement. The pressure can serve as a motive force for transferring solution(s) from/to different locations in the device or system, and also as a means to control rotational velocity of a layer. When driving multiple solutions, the movement can simultaneously connect a first fluid path(s) and generate pressure for fluidic transfer, where the pressure is vented after fluidic transfer, then another movement can disconnect the first fluid path, connect a second fluid path and generate additional pressure for fluidic transfer.

One aspect of the invention provides sample preparation modules comprising a housing having an interior surface, a central shaft comprising a threaded section, wherein the central shaft rotates relative to the housing, a pressure cap and a plurality of coaxially arranged layers, at least one layer being a rotor and one being a stator (relative to the rotor).

In some embodiments, provided herein is a method of regulating fluid flow in a device, comprising providing a device comprising a pressure chamber, said pressure chamber comprising a pressure cap configured to increase or decrease the volume of the pressure chamber; a plurality of coaxially arranged layers, at least one being a rotor and one being a stator, said coaxially arranged layers having complementary facing surfaces assembled in a frictional, sealed engagement, each layer having at least one passageway with an upstream entry and a downstream outlet capable for successive selective placement in communication to establish plural dedicated flow paths within the assembly; and a central shaft, said central shaft engaged with the rotor layer, wherein said central shaft comprises threads engaged with the pressure cap to increase or decrease the volume of the pressure chamber, thereby increasing or decreasing pressure in the pressure chamber, and wherein rotation of said central shaft simultaneously rotates the rotor layer while increasing or decreasing pressure in the pressure chamber; loading a sample into a coaxially arranged layer of the device, wherein the rotor layer is in a first position in which all fluidic paths are occluded; and applying torque to the central shaft via a driving module engaged with the central shaft, wherein rotation of the central shaft also moves the pressure cap to reduce the volume of the pressure chamber, thereby increasing pressure in the pressure chamber, said increased pressure slowing the rate of rotation of the rotor layer until said rotor layer reaches a second position, wherein the second position provides an uninterrupted fluidic path, thereby pumping a fluid from a first coaxially arranged layer to a second coaxially arranged layer, and subsequently venting the pressurized pressure chamber, thereby reducing pressure after transfer of said fluid, increasing the rate of rotation of the rotor layer.

In some embodiments, the method of regulating fluid flow in a device further comprises rotating the rotor layer to a subsequent position by applying torque to the central shaft via a driving module engaged with the central shaft, wherein rotation of the central shaft also moves the pressure cap to reduce the volume of the pressure chamber, thereby increasing pressure in the pressure chamber, said increased pressure slowing the rate of rotation of the rotor layer until said rotor layer reaches the subsequent position, and wherein the subsequent position provides an uninterrupted fluidic path between two coaxially arranged layers, and wherein the fluidic path is linked to the pressure chamber so that the increased pressure in the pressure chamber pumps a fluid along the fluidic path, and subsequently vents the pressurized chamber, thereby reducing pressure after transfer of the fluid, increasing the rate of rotation of the rotor layer. In some embodiments, the subsequent position is a third, fourth, fifth, sixth, seventh, eighth, ninth, or tenth position.

In some embodiments, the fluid comprises the sample. In some embodiments, the fluid comprises a reagent. In some embodiments, the reagent is a lysis reagent, an extraction reagent, a purification reagent, an amplification reagent, or a detection reagent. In some embodiments, the sample is loaded into said at least one passageway of said coaxially arranged layer.

In some embodiments, the applied torque is constant. In some embodiments, the driving module comprises a constant torque spring. In some embodiments, the rotor stops at said second position, restarting after said fluid has transferred from said first coaxially arranged layer to said second coaxially arranged layer. In some embodiments, the angular velocity of said rotor layer is less than 90%, 80%, 70%, 60%, 50%, 40%, 30%, 20%, 10%, 9%, 8%, 7%, 6%, 5%, 4%, 3%, 2%, or 1% of the initial velocity of the rotor layer at equilibrium pressure when the rotor layer reaches the second position. In some embodiments, the rotor stops at said subsequent position, restarting after said fluid has transferred from said first coaxially arranged layer to said second coaxially arranged layer. In some embodiments, the angular velocity of said rotor layer is less than 90%, 80%, 70%, 60%, 50%, 40%, 30%, 20%, 10%, 9%, 8%, 7%, 6%, 5%, 4%, 3%, 2%, or 1% of the initial velocity of the rotor layer at equilibrium pressure when the rotor layer reaches the subsequent position.

In some embodiments, the device comprises a reagent layer comprising at least one reagent solution. In some embodiments, the reagent layer is the rotor layer. In some embodiments, the reagent layer is the first layer. In some embodiments, the reagent layer comprises a holding chamber. In some embodiments, the reagent layer comprises a surface on the interior of said pressure chamber. In some embodiments, the reagent layer comprises lysis buffer, a wash buffer, and an elution buffer, each buffer occupying a separate passageway in said reagent layer.

In some embodiments, the rotor layer is the first layer. In some embodiments, the rotor layer is the second layer. In some embodiments, the said rotor layer comprises a reagent solution. In some embodiments, the second layer comprises a collection chamber, an analytic chamber, or a detection chamber. In some embodiments, at least one of said coaxially arranged layers comprises a pre-loaded reagent. In some embodiments, the pressure chamber comprises a pre-loaded reagent. In some embodiments, the pre-loaded reagent is lyophilized.

Also provided herein is an automated device, comprising: a housing having an interior surface; a central shaft comprising a threaded section, wherein the central shaft rotates relative to the housing; a constant force spring engaged with said central shaft, wherein said constant force spring generates torque to rotate said central shaft; a pressure cap comprising a gasket capable of engaging with the interior surface of the housing to create an airtight seal and a central column having threads, wherein the threads engage the threaded section of the central shaft; and a plurality of coaxially arranged layers, at least one being a rotor and one being a stator, said coaxially arranged layers having complementary facing surfaces assembled in a frictional, sealed engagement, each layer having at least one passageway with an upstream entry and a downstream outlet capable for successive selective placement in communication to establish plural dedicated flow paths within the assembly, wherein rotation of said rotor selectively connects passageways of different coaxial layers, thereby serially forming and disrupting a plurality of fluid paths, wherein a first coaxially arranged layer engages with the interior surface of the housing to create an airtight seal, the surface of the first layer with the housing and the pressure cap thereby forming a compartment, wherein rotation of the central shaft relative to the housing compresses the compartment, thereby generating pressure against the upstream surface of the first coaxially arranged layer; and wherein rotation of the central shaft relative to the housing decreases in angular velocity due to increased pressure in said compartment until said rotor aligns with said stator to generate an aligned path for release of said pressure in said compartment.

In some embodiments, the aligned path provides a fluidic path for transfer of a fluid from a passageway in the rotor layer through a passageway in the stator layer. In some embodiments, the aligned path further provides a path for air pressure to be released from said compartment, thereby increasing the angular velocity of the rotation of the central shaft.

A method of detecting a target nucleic acid in a sample, comprising: providing an integrated device comprising a pressure chamber, an extraction module, an amplification module, and a driving module; adding a sample to said extraction module; initiating said driving module to generate torque to rotate a central shaft, thereby initiating isolation of nucleic acids from said sample and transfer of said nucleic acids to an amplification module, wherein said rotation results in increased pressure in said pressure chamber, thereby increasing resistance to said rotation until a fluid is transferred along a fluidic path created by said rotation, thereby alleviating said pressure through venting of said pressure chamber along the fluidic path; and detecting the presence or absence of a target nucleic acid in said amplification module.

In some embodiments, the integrated device comprises a plurality of coaxially-arranged layers, and wherein friction between said coaxially-arranged layers affects said resistance to said rotation of the central shaft. In some embodiments, the diver module comprises a constant torque spring motor. In some embodiments, the detection comprises performing an amplification reaction. In some embodiments, the amplification reaction is RT-LAMP. In some embodiments, the amplification module comprises lyophilized reagents.

In some embodiments, provided herein is a device (e.g., a microfluidic device, e.g., for sample preparation, sample treatment, sample volume quantification, and/or sample analysis) comprising a spring that can be used to actuate mobile parts in a device, including but not limited to: an integrated device, a multicomponent device or a SlipChip device.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

Described herein are a number of devices and methods that can be used individually or in various combinations for applications including but not limited to those listed herein. Furthermore, they can be used in various combinations with previously disclosed devices and methods for previously described applications.

The present application incorporates the following applications by reference in their entireties for any and all purposes: U.S. Application 61/262,375, “Slip Chip Device and Methods,” filed on Nov. 18, 2009; U.S. Application 61/162,922, “Slip Chip Device and Methods,” filed on Mar. 24, 2009; U.S. Application 61/340,872, “Slip Chip Device and Methods,” filed on Mar. 22, 2010; international application PCT/US2010/028361, “Slip Chip Device and Methods,” filed on Mar. 23, 2010; U.S. Application 61/516,628, “Digital Isothermal Quantification of Nucleic Acids Via Simultaneous Chemical Initiation of Recombinase Polymerase Amplification (RPA) Reactions on Slip Chip,” filed on Apr. 5, 2011; U.S. Application 61/518,601, “Quantification of Nucleic Acids With Large Dynamic Range Using Multivolume Digital Reverse Transcription PCR (RT-PCR) On A Rotational Slip Chip Tested With Viral Load,” filed on May 9, 2011; U.S. application Ser. No. 13/257,811, “Slip Chip Device and Methods,” filed on Sep. 20, 2011; U.S. application Ser. No. 13/440,371, “Analysis Devices, Kits, And Related Methods For Digital Quantification Of Nucleic Acids And Other Analytes,” filed on Apr. 5, 2012; U.S. application Ser. No. 13/467,482, “Multivolume Devices, Kits, Related Methods for Quantification and Detection of Nucleic Acids and Other Analytes,” filed on May 9, 2012; U.S. application Ser. No. 13/868,028, “Fluidic Devices and Systems for Sample Preparation or Autonomous Analysis,” filed on Apr. 22, 2013; U.S. application Ser. No. 13/868,009, “Fluidic Devices for Biospecimen Preservation,” filed on Apr. 22, 2013; international application PCT/US2013/0376, “Fluidic Devices for Biospecimen Preservation,” filed on Apr. 22, 2013; international application PCT/US2013/0376, “Fluidic Devices and Systems for Sample Preparation or Autonomous Analysis,” filed on Apr. 22, 2013; U.S. application Ser. No. 13/869,856, “Slip-Induced Compartmentalization,” filed Apr. 24, 2013; U.S. Provisional Application No. 61/856,155, filed Jul. 19, 2013, and the benefit of U.S. Provisional Application No. 61/880,399, filed Sep. 20, 2013; international application PCT/US2013/63594, “Methods and Systems for Microfluidics Imaging and Analysis,” filed on Oct. 4, 2013; U.S. Provisional Application No. 61/969,008, filed Mar. 21, 2014; international application PCT/US2014/0347, “Parallelized Sample Handling,” filed on Apr. 18, 2014; international application PCT/US2014/0470, “Digital Assay for Quantifying and Concentrating Analytes,” filed on Jul. 17, 2014; U.S. Application 62/038,036, “The Pumping Lid: Devices and Methods for Programmable Generation of Positive and Negative Pressures,” filed on Aug. 15, 2014; U.S. Application 62/050,647, “Digital Microfluidics Methods for Optimizing Isothermal Amplification Reactions,” filed on Sep. 15, 2014; international application PCT/US2014/056401, “System and Method for Movement and Timing Control,” filed on Sep. 18, 2014; International Application No. PCT/US2014/060977 “Enhanced Nucleic Acid Identification and Detection” filed on Oct. 16, 2014, and U.S. Application 62/075,648, “Microfluidic Measurements of the Responses of an Organism to a Drug, filed on Nov. 5, 2014; U.S. Application 62/096,131 “Devices and Methods for Autonomous Measurements,” filed on Dec. 23, 2014; U.S. Application 62/104,578, “Devices and methods of sensitive detection, analysis and quantification with wide dynamic range,” filed on Jan. 16, 2015, and U.S. Application 62/108,443, “Devices and Methods for Nucleic Acid Modification and Analysis,” filed Jan. 27, 2015.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an exemplary schematic of the alignment of two layers of a device.

FIG. 2A shows an exemplary exploded side view schematic of a device comprising a pressure cap.

FIG. 2B shows an exemplary exploded three-quarters view schematic of a device comprising a pressure cap and plurality of coaxially arranged layers.

FIG. 3 shows an exemplary automated actuation mechanism comprising a constant torque spring motor to actuate the movement of layers and or affect pressure in the automated device.

FIG. 4 depicts the use of a constant torque spring to generate pressure in a cavity, by using an actuator to pull on a threaded pressure cap.

FIG. 5 shows representative graphs of the expected trends for the angle of rotation (theta), pressure in the cavity and angular speed over time for the system described in FIG. 4, according to the equations of the model.

FIG. 6 depicts the effect of change in pressure in a chamber of the integrated device on the rotation of a moving component (e.g., an actuator) by the constant torque spring to control speed or rotation or movement in a device.

FIG. 7 diagrams an example of combination of pressure and venting to control speed of rotation of a moveable part in a device.

FIG. 8 diagrams an example of the combination of pressure and liquid pumping to control speed of rotation of a moveable part in a device.

FIG. 9 shows a graph of rotation angle over time in an extraction process controlled by a spring motor interacting with a moveable element that controls a pressure cap to apply pressure, and pumping and venting due to rotation of a layer. The traces show the predicted value for the rotation angle (θ(t)), rotation speed (ω(t)) and pressure in the cavity over time. Dashed lines indicate the moment in which pumping and venting happens for two different overlap positions due to the rotating layer.

FIG. 9B depicts an example of design for multistep processes requiring the pumping of lysed sample, washing buffer, and pump elution buffer at different times. The rotation angle versus time is reported in the graph. The shaded areas represent the layer rotation angle regions in which the holes overlap, and flow can be produced.

FIG. 10 depicts an example of a pressure chamber in a device for generating flow using the pressure produced by the constant torque approach.

FIG. 11A shows an example of the use of mixing chambers to mix a lysis buffer with a sample.

FIG. 11B shows an example of the use of mixing chambers to mix a sample with an amplification reagent.

FIG. 12 depicts photographs of sample blister packs for use with an reagent layer of the automated device disclosed herein.

FIG. 13 depicts a schematic of an embodiment of a two layer device for multiplex amplification in a loading position and an amplification position.

FIG. 14 is a photograph of an embodiment of a two layer device for multiplex amplification fabricated by 3D printing, showing the device in a loading position and an amplification position

FIG. 15 shows an example of layers of an amplification module for digital/single molecule detection and analyte quantification

FIG. 16 shows an example of an amplification module and positions for loading and mixing.

FIG. 17 depicts one example of an integrated device for detection of target nucleic acid sequences.

FIG. 18 shows a cross-sectional view of an embodiment of an integrated device comprising a pumping lid, blisters for reagent storage, a nucleic acid extraction module, an amplification module, a heating module, and a driving module.

FIG. 19 shows the interface between the base station comprising a driving module and other modules in the device. Separation is part of one method to detect reaction products.

FIG. 20 shows a photograph of modules of an embodiment of the device fabricated with 3D printing and having the same geometry as the embodiments depicted in FIG. 17-FIG. 19.

FIG. 21 depicts features of a nucleic acid extraction module for performing the extraction and for integration with other modules.

FIG. 22A depicts an example of a heating module with a temperature regulated by paraffin wax.

FIG. 22B shows a diagram of one embodiment of a heating module and circuit used for temperature control using a thermal switch.

FIG. 22C shows an embodiment of the heating module comprising an integrated circuit board and battery for heating an amplification module.

FIG. 23 shows the use of multiple materials of different rigidity in layers of an embodiment of the device or module.

FIG. 24 shows an embodiment of a 3D design of an integrated device including a DNA/RNA extraction module and a pumping lid. Panel 1 represents the designs of the 3 layers (components) that make up the DNA/RNA extraction module, and the middle layer (component) represents the rotating layer. Panel 2 shows an exploded view of how the abovementioned 3 layers (components) are assembled. Panel 3 shows the assembled DNA/RNA extraction module on the left-hand side and the pumping lid on the right-hand side.

FIG. 25 shows photographs of a manufactured integrated device according to the design of FIG. 24, the integrated device comprising a constant torque spring motor in a driving module, a DNA/RNA extraction module, and a pumping lid.

FIG. 26 shows photographs demonstrating pumping of food dye liquid solution by an integrated device.

FIG. 27 shows components for leak-proof attachment of an extraction module to an amplification module.

FIG. 28 shows photos of the operation of the integrated device to transfer liquids from a nucleic acid extraction module to an amplification module.

FIG. 29 is a diagram representing a cross-sectional view of a sample preparation and amplification module.

FIG. 30 shows a graph of amplification results of 16S rRNA from C. trachomatis at different concentrations for purification by an integrated device (Device) as compared to a standard protocol (Off-device) (Panel A). Also shown is a bar chart depicting the amplification results of 16S rRNA from C. trachomatis at 24 IFU/mL for purification by an integrated device (Device) as compared to a standard protocol (Off-device) (Panel B)

FIG. 31 shows the results of amplification of Chlamydia trachomatis (CT) and Neisseria gonorrhoeae (NG) 16S rRNA (Panels A and B) and amplification using primers alone or primers with the incorrect target to show cross reactivity (Panels C and D)

FIG. 32 shows the results of amplification reactions using lyophilized RT-LAMP reagents.

FIG. 33 is a photograph of the results of LAMP amplification performed in an embodiment of the multiplex amplification device shown. Fluorescent signal from the LAMP amplification is detected by stereoscope, and a cell phone image shows an embodiment of the device with the amplified product.

FIG. 34 shows the results of multiplex detection of Chlamydia trachomatis and Neisseria gonorrhoeae 16S RNA in an embodiment of the multiplex amplification device, including negative controls.

### DETAILED DESCRIPTION OF THE INVENTION

Unless defined otherwise, all technical and scientific terms used herein have the same meaning as is commonly understood by one of skill in the art to which the claimed subject matter belongs. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, the singular forms “a,” “an” and “the” include plural referents unless the context clearly dictates otherwise. In this application, the use of the singular includes the plural unless specifically stated otherwise. As used herein, the use of “or” means “and/or” unless stated otherwise. Furthermore, use of the term “including” as well as other forms (e.g., “include”, “includes”, and “included”) is not limiting.

As used herein, ranges and amounts can be expressed as “about” a particular value or range. About also includes the exact amount. Hence “about 10 degrees” means “about 10 degrees” and also “10 degrees.” Generally, the term “about” can include an amount that would be expected to be within experimental error.

The invention provides devices and methods for preparing, processing, storing, preserving, and/or analyzing samples. In particular, such devices allow for multiple reactions to be performed while minimizing contamination. Described herein are structural features for such devices, as well as methods for their use in sample preparation or storage.

### Overview

Disclosed herein are devices and systems for managing fluid flow, mixing, and reactions in an automated device. In some embodiments, the devices provided herein are actuated by a motor (e.g., a spring motor) providing torque. The torque provides rotation that is inhibited by increased pressure and friction. In some embodiments, the pressure and friction is balanced with the torque to provide controlled transfer of fluids in multi-step reactions. In some embodiments, provided herein are automated integrated devices comprising a driving module that rotates a shaft engaged with a layer in the device, and a pressure cap. The shaft is engaged with the layer to rotate the layer relative to a stationary layer, while the shaft is engaged with the pressure cap to increase pressure in a chamber.

The devices of the invention can include one or more structural features, such as one or more additional layers, a chamber (e.g., a well, a channel, a hole, a bridge, or a cavity, or any described herein), or a capture region. In particular, the chamber can be completed or partially enclosed (e.g., such as in an enclosed channel) or be open (e.g., such as in a well). The various structures described herein can have any useful dimension, cross-section, planarity, or surface characteristic. Any of the devices described herein can be used individually or in combination with the devices or with one or more features of the devices described in, e.g., U.S. Pub. Nos. 2006-0003439; 2007-0172954; 2010-0078077; 2010-0233026; 2011-0112503; 2011-0142734; 2011-0165037; 2011-0176966; 2011-0177586; 2012-0329171; and 2013-0309679; U.S. Pat. Nos. 7,129,091; 7,655,470; 7,901,939; 8,304,193; 8,273,573; and 8,329,407; U.S. patent application Ser. No. 13/648,922, filed Oct. 10, 2012; Int. Pub. Nos. WO 2004/038363; WO 2009/149257; WO 2008/079274; WO 2006/101851; and WO 2015/160419; and U.S. Provisional Pat. Appl. Nos. 60/379,927; 60/394,544; 60/585,801; 60/623,261; 60/763,574; 60/875,856; 60/881,012; 60/899,449; 60/930,316; 60/936,606; 60/962,426; 61/130,930; and 61/335,570. Further, any of these devices can be used in any method described herein, as well as those methods described in the above-mentioned U.S. Pat. Nos., U.S. Pub. Nos., U.S. Pat. Appl. No., Int. Pub. Nos., and U.S. Provisional Pat. Appl. Nos., which are incorporated herein by reference.

The devices and methods described herein can be used to perform processes for sample preparation and sample preservation, including liquid sample storage and preservation. A liquid sample (such as blood, saliva, urine, blood plasma, serum, purified protein or nucleic acid solution, cell culture medium, environmental sample etc., or any other described herein) can be loaded in the device. A sample, either before or after processing or analysis, as well as any substance described herein (e.g., a reagent, a buffer, etc.) can be preserved or stored either in the dry state or in the liquid state. In some instances, the sample is a liquid sample, and preservation in the liquid state may be preferable. In other instances, the sample is a liquid sample intended for long term storage (e.g., more than six months) and/or for storage at high temperatures (e.g., more than about 4° C.), and preservation in the dry state may be preferable. In yet other instances, the sample is a dried liquid sample (e.g., a dried blood spot, such as for DNA analysis, clinical testing, or any analysis described herein).

The devices and systems of the invention can be used to quantify volumes of a sample, a reagent, or any useful substance (e.g., any described herein). In particular, quantification of volumes can be used in combination with any of the other devices and methods described herein, such as for sample preservation, sample treatment, sample preparation, and/or sample analysis. In particular, such volume quantification techniques can be useful for screening of special populations (such as newborns, infants, or small animals, e.g., for screening inherited metabolic disorders or lysosomal storage disorders, such as Fabry, Gaucher, Krabbe, Niemann-Pick A/B, and Pompe disease; for screening viral infections, such as HIV or CMV; or for screening other disorders using useful diagnostic markers, such as screening for succinylacetone, acylcarnitines, and amino acids to detect tyrosinemia type I (TYR 1) in newborns or infants), for use with a dried blood spot (DBS) sample (e.g., in combination with one or more sample preservation and/or storage devices and methods, as described herein), for screening metabolites (e.g., for pharmacokinetic, pharmacodynamic, toxicokinetic, or other drug monitoring assessments), for use in clinical trials (e.g., for pharmacokinetic or pharmacodynamic assessment of investigational drugs in clinical trials), and for determining adherence with particular drugs (e.g., for pharmacokinetic, pharmacodynamic, toxicokinetic, or other drug monitoring assessments).

The device of the present invention can be used to study and perform a number of assays, including but not limited to coagulation or clotting assays, protein aggregation, protein crystallization (including the use of lipidic cubic phase), crystallization and analysis of small molecules, macromolecules, and particles, crystallization and analysis of polymorphs, crystallization of pharmaceuticals, drugs and drug candidates, biomineralization, nanoparticle formation, the environment (via aqueous and air sampling), culturing conditions (e.g., stochastic confinement, lysis of cells, etc.), drug susceptibility, drug interactions, high throughput screening (e.g., one first substance with many, different second substances, or many, different first substances with many, different second substances), multiplex assays (e.g. PCR, Taqman, immunoassays (e.g., ELISA, FISH, etc.)), amplification (e.g., flow through immunoassays (e.g. that use capture reagents or, capture regions), digital-single molecule ELISA and digital immunoassays, amplification (e.g., PCR, ligase chain reaction (LCR), transcription mediated amplification (TMA), reverse transcriptase initiated PCR, DNA or RNA hybridization techniques, sequencing, and the like), sandwich immunoassays, chemotaxis assays, ramification amplification (RAM), etc. Exemplary techniques for blood assays, crystallization assays, protein aggregation assays, culturing assays are described in U.S. Pat. Nos. 7,129,091, 6,949,575, 5,688,651, 7,329,485, 6,949,575, 5,688,651, 7,329,485, and 7,375,190; U.S. Pub. Nos. 2007/0172954, 2006/0003439, 2003/0022243, and 2005/0087122; and Int. Pub. Nos. WO 2007/089777 and WO 2009/015390, each of which is incorporated herein by reference in its entireties. The device of the present invention can be used for various syntheses, including catalysis, multistep reactions, immobilized multistep synthesis (e.g., small molecule, peptide and nucleic acid syntheses), solid state synthesis, radioisotope synthesis, etc. Finally, the device of the present invention can be used for purification and enrichment of samples.

### Devices

Generation of Pressure and Modification of Fluid Paths

Disclosed herein are devices and systems for creating and disrupting fluid paths and for generating pressure using a single movement. The pressure can serve as a motive force for transferring solution(s) or other fluid(s) from/to different locations in the device or system. When driving multiple solutions, the movement can simultaneously connect a first fluid path(s) and generate pressure for fluidic transfer; then another movement can disconnect the first fluid path, connect a second fluid path and generate additional pressure for fluidic transfer.

One implementation provides devices for simultaneously modifying fluid paths and generating pressure, said device comprising a pressure cap and a plurality of coaxially arranged layers, at least one being a rotor and at least one being a stator, said coaxially arranged layers having complementary facing surfaces assembled in a frictional, sealed engagement, each layer having at least one passageway with an upstream entry and a downstream outlet capable for successive selective placement in communication to establish plural dedicated flow paths within the assembly, wherein a first coaxially arranged layer engages with the pressure cap to create an airtight compartment that is compressible upon rotation of the rotor, and wherein rotation of said rotor selectively connects passageways of different coaxial layers thereby serially forming and disrupting a plurality of fluid paths.

Another implementation provides devices comprising a housing having an interior surface, a central shaft comprising a threaded section, wherein the central shaft rotates relative to the housing, a pressure cap, and a plurality of coaxially arranged layers.

Preferably the pressure cap comprises a gasket capable of engaging with the interior surface of the housing to create an airtight seal and a central column having threads, wherein the threads engage the treaded section of the shaft. The first coaxially arranged layer can engage with the interior surface of the housing to create an airtight seal, the surface of the first layer with the housing and pressure cap thereby forming a compartment, wherein rotation of the central shaft relative to the housing compresses the compartment by pulling down the pressure cap, thereby generating pressure against the upstream surface of the first coaxially arranged layer.

At least one of the plurality of coaxially arranged layers is a rotor. One or more of the remaining layers is a stator (relative to the rotor). The rotor(s) can be engaged directly or indirectly with the central shaft, whereas the stator(s) can be engaged directly or indirectly with the housing. In certain implementations, the stator is permanently affixed to the housing, thus acting effectively as a base for the housing. Alternatively, the stator(s) can be engaged directly or indirectly with the central shaft, whereas the rotor(s) can be engaged directly or indirectly with the housing. Thus, when the central shaft and housing rotated relative to one another, the rotor(s) and stator(s) also rotate relative to one another. The coaxially arranged layers have complementary facing surfaces and are assembled in a frictional, sealed engagement, each layer having at least one passageway with an upstream entry and a downstream outlet capable for successive selective placement in communication to establish plural dedicated flow paths within the assembly. During operation rotation of said rotor selectively connects passageways of different coaxially layers thereby serially forming and disrupting a plurality of fluid paths.

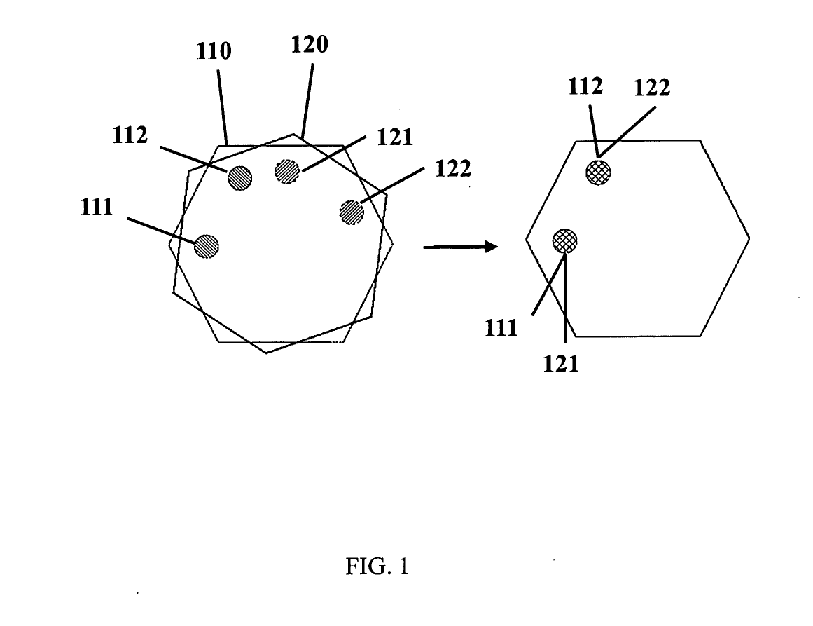

The devices of the invention can include layers arranged (e.g., coaxially) to allow for connection and disconnection of one or more fluid paths by movement of the layers (e.g., rotational movement). For example, in a first position, one or more passageways (e.g., 111, 112) in a first layer (e.g., 110) are not connected to (e.g., are not in fluidic communication with) one or more passageways (e.g., 121, 122) in a second layer (e.g., 120). Upon moving the first layer relative to the second layer, the downstream outlets of one or more passageways of the first layer align with the entries of the one or more passageways of the second layer and a connection is formed (see, e.g., FIG. 1). This movement can be accomplished by rotating the first layer having the first passageway relative to the second layer. Alternatively, this movement can include rotating the second layer having the second passageway relative to the first layer.

Devices can contain fluidic elements or structures, such as channels, reservoirs, chambers, blister packs, wells, filters, membranes, one-way valves, and sample tubes. The dimensions of any structure (e.g., one or more channels) may be chosen to maintain a particular volumetric or linear flow rate of a fluid in the device. For example, choice of such dimensions can be useful to control the filling of the device with particular fluids or the flow rate of such fluids through pathways, filters and/or wells or aeration to effect mixing of liquids.

The wells, channels, reservoirs, chambers, tubes or other structure can include any useful cross-section. Cross-sections can be of any useful shape (e.g., rectangular, square, circular, oval, trapezoidal, triangular, or irregular cross-sections). Cross-section shape or dimensions can vary along the axis of any structure. For instance, when the structure is a channel, the cross-section of the channel along the axis of fluid flow can change from one cross-sectional shape to another, such as from a circular to a rectangular cross-section. In another instance, the dimensions of the cross-section can be uniform or can vary along any axis, such as a channel that tapers or expands along the axis of fluid flow.

The housing, shaft, coaxially arranged layers, and pressure cap can be formed from any useful material or combination of materials. The materials used to form the components of devices of the invention are selected with regard to physical and chemical characteristics that are desirable for proper functioning of the component. Suitable, non-limiting examples of materials include plastics (e.g., cyclic olefin copolymer (COC), cyclic olefin polymer (COP), polyethylene terephthalate (PET), polyethylene (PE), high-density polyethylene (HDPE), low-density polyethylene (LDPE), polyvinyl chloride (PVC), polypropylene (PP), polystyrene (PS), high impact polystyrene (HIPS), polyamides (PA), acrylonitrile butadiene styrene (ABS), polyethylene/acrylonitrile butadiene styrene (PE/ABS), polycarbonate (PC), polycarbonate/acrylonitrile butadiene styrene (PC/ABS), polymethylmethacrylate (PMMA)), metals, elastomers (e.g., polydimethylsiloxane (PDMS)), glass (e.g., borosilicate), ceramics, and composite materials, such as carbon fiber composites. In some cases, devices are formed from metal or other magnetic materials.

The device or components thereof can be formed by any useful process, including but not limited to molding (e.g., injection molding, vacuum molding, or over-molding), machining (e.g., drilling, milling, or sanding), embossing (e.g., hot embossing) and etching (e.g., laser, deep reactive ion etching, KOH etching, or HF etching). In microfluidic applications, the layers can be fabricated from a material that enables formation of high resolution features such as microchannels, chambers, mixing features, and the like, that are of millimeter, micron, or submicron dimensions (e.g., PDMS, PMMA, glass). Applicable microfabrication techniques include but are not limited to dry etching, wet etching, laser etching, laser ablation, molding, embossing, photolithography, soft lithography, lamination or the like.

Pressure Cap

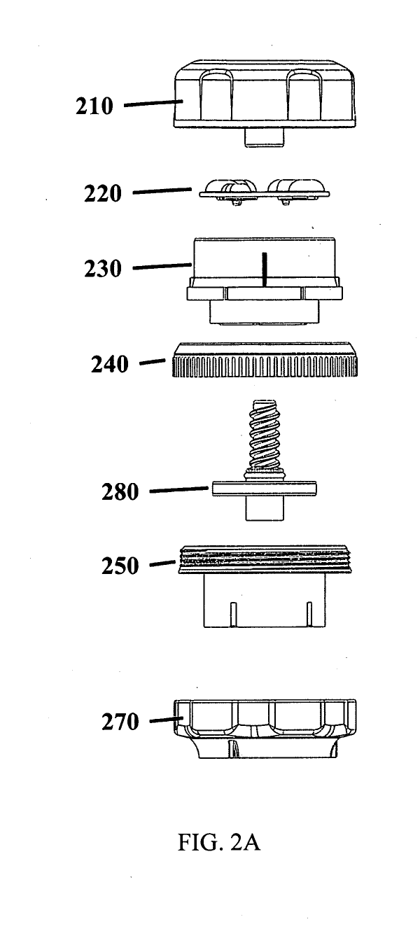

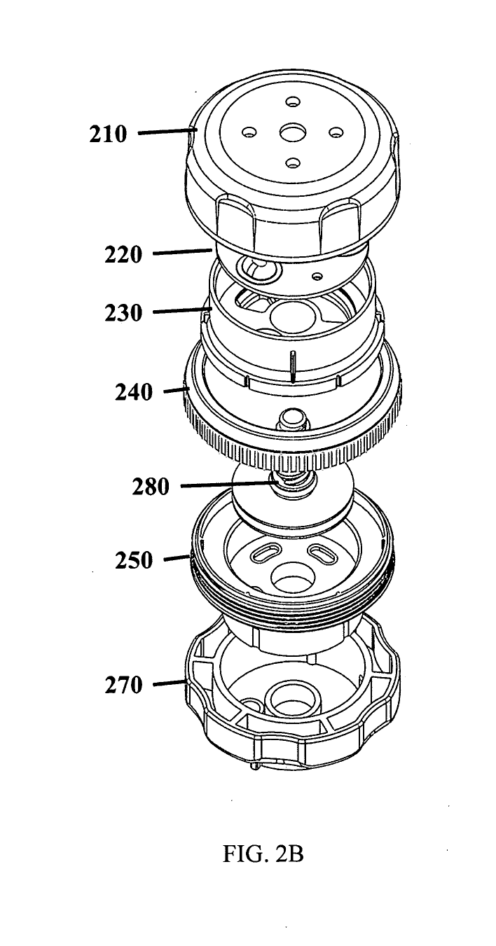

A rotational device can be configured to increase pressure within the device as the cap is rotated. This pressure can be generated by the decreasing internal volume of a compartment within the device as the cap lowers toward the base. In some examples, such a device comprises a center shaft (e.g., 280, FIG. 2A/2B) with a thread, allowing a central column on the cap to thread onto the central shaft and screw down with rotation. In other examples, an outer part of the housing or base can comprise threads onto which the cap can thread and screw down with rotation. In some cases, one or more posts, knobs, grooves, guiderails, or other features can guide the motion of the cap. In one implementation, the pressure cap comprises a key or keyseat that engages with a complementary keyseat or key in the housing thereby inhibiting rotation of the pressure cap relative to the housing. The motion of the cap can be characterized by a smooth motion of combined rotation and downward motion relative to the base. In other examples, the motion of the cap can be characterized by variable rate motion, e.g. a first 15° rotation resulting a quarter-inch downward motion of the cap and a second 15° rotation resulting in a half-inch downward motion of the cap.

FIGS. 2A and 2B show exemplary schematics of a device with a pressure cap and various layers. Device components can include but are not limited to a pressure cap 200, a housing 210, a reagent layer 220, a reagent layer storage 230, a separation layer 240, a receiving layer 250, a central threaded shaft 280, an analysis layer 260, a base 270, and a threaded connector 290 adapted to hold other components in place. Not all of these components are required to be present in a particular device. One or more of the layers can include heating, such as the reagent storage layer and/or the receiving layer. Layers can fulfill multiple functions; for example, a layer can comprise a membrane to serve as a separation layer and also comprise a well to serve as a receiving layer. Additionally, a single structure can comprise both a layer and additional structures, such as a housing or shaft. In a preferred implementation, the device comprises reagent layer, a separation layer and a receiving layer.

The housing can be formed as a single structure or can be comprised of two or more segments that, when assembled, form an airtight unit. In some implementations, the housing is permanently affixed to the first (i.e. most upstream) layer of the device. The permanent fixation can be achieved by forming the housing and first layer from a single material, e.g., co-molded in plastic. Alternatively, the housing and first layer can be permanently attached using any adhesive, preferably air and fluid resistant, known in the art. In those implementations in which the housing is formed of two or more segments, one or more of those segments can be permanently affixed to a coaxially arranged layer. In certain implementations, each of the two or more segments of the housing are permanently affixed to a coaxially arranged layer.

Pressure generated by the cap can be used to drive fluid flow within the device. For example, solution can be impelled through a matrix or filter, or air can be driven to dry a matrix or filter. The graduated application of pressure produced by a smooth motion of the cap can reduce the risk of leakage compared to a sudden application of pressure.

The cap can generate pressures of at least about 1 kilopascal (kPa), 2 kPa, 3 kPa, 4 kPa, 5 kPa, 6 kPa, 7 kPa, 8 kPa, 9 kPa, 10 kPa, 15 kPa, 20 kPa, 25 kPa, 30 kPa, 35 kPa, 40 kPa, 45 kPa, 50 kPa, 55 kPa, 60 kPa, 65 kPa, 70 kPa, 75 kPa, 80 kPa, 85 kPa, 90 kPa, 95 kPa, 100 kPa, 110 kPa, 120 kPa, 130 kPa, 140 kPa, 150 kPa, 160 kPa, 170 kPa, 180 kPa, 190 kPa, 200 kPa, 210 kPa, 220 kPa, 230 kPa, 240 kPa, 250 kPa, 260 kPa, 270 kPa, 280 kPa, 290 kPa, 300 kPa, 310 kPa, 320 kPa, 330 kPa, 340 kPa, 350 kPa, 360 kPa, 370 kPa, 380 kPa, 390 kPa, 400 kPa, 410 kPa, 420 kPa, 430 kPa, 440 kPa, 450 kPa, 460 kPa, 470 kPa, 480 kPa, 490 kPa, or 500 kPa.

The cap can generate a number of distinct pressures (e.g., for particular fluid operations or steps), including 1, 2, 3, 4, 5, 6, 7, 8, 9, 10, 11, 12, 13, 14, 15, 16, 17, 18, 19, 20, 25, or more distinct pressures.

In some cases, actuation of the device can create a vacuum or negative pressure in one region of the device relative to the rest of the device. For example, actuation of the device can cause actuation of piston to drop down and create a reduced or negative pressure, such as in a passageway or below a filter. Alternatively, the direction of rotation can be reversed, thereby expanding the compartment formed by the pressure cap and thus creating a negative pressure. Such negative pressure can be used to pause or reverse fluid flows. In certain implementations, the direction of rotation can be alternated between clockwise and counter-clockwise to generate a back and forth flow to, e.g. mix solutions or dissolve a solid reagent deposited in a passageway. In another implementation, the actuation of the device can cause shape change of a buckle pump or diaphragm. Locally different pressures can be achieved in different sections of the device through the use of one-way or check valves.

Coordinated pressurization and rotation can simplify the operation or manipulation of the device. The device can be driven by a single rotational movement, and the switching of fluidic path and pressurization can be achieved simultaneously. The device can be fully autonomous driven by a single movement powered by springs, or motors. By being coordinated, the present device can provide more reliability than systems requiring a plurality of motive forces, since the failure of any of the plurality of forces would render the device useless.

The motion of the layers can be around a shared motive axis. The motion around the motive axis can be around a central axis. An axis can comprise a single shaft. An axis can comprise multiple linked shafts.

A shaft can comprise threads (e.g., on its external surface) or other structures for engaging with another surface. A shaft can engage with another surface, such as a pressure cap. For example, threads on the external surface of a shaft can engage with threads on the internal surface of a pressure cap sleeve. Threads can extend around the full circumference of the central shaft, or there can be breaks in the threading around the shaft circumference. The surface with which the central shaft engages, such as the pressure cap sleeve, can have threads extending around the full or only a portion of its circumference. In one implementation, the externally threaded section of a shaft is distal to the location of engagement of the central shaft with a drive. In other implementations the pressure cap comprises external threads on cap and the shaft comprises internal threads along at least a portion of the length of the shaft.

Threads can have variable pitch, which can result in a variable compression per degree of rotation of a pressure cap. For example, the thread pitch can change along the length of a shaft, cap, or other surface. Similarly, the pitch of the threads of the cap need not be the same as the pitch of the shaft or other surface with which the cap's threads engage. For example, the threads of the central column of the pressure cap can have a different pitch that the threaded section of the shaft.

In some cases, the shaft comprises a plurality of linked sections, with each section engaged with its neighboring (e.g., proximal or distal) sections to form an arrangement of stacked shafts. Shaft sections can be linked directly, such that each section rotates at the same rate. Shaft sections can be linked via a geared linkage. Geared linkages can enable different rates of rotation between neighboring shaft sections. Rotation between neighboring shaft sections can be characterized by proportional movement, non-proportional movement, or discontinuous movement.

Rotatable layers can be engaged directly with a shaft or shaft section. Rotatable layers can be engaged indirectly with a shaft or shaft section, such as by engagement mediated via another rotatable layer, or by engagement with external threads on a surface such as a pressure cap sleeve. A rotatable layer can be permanently attached to a shaft.

The angular rotation of a layer can be the same as the angular rotation of a shaft (e.g., central shaft). Alternatively, the angular rotation of a layer can be different from the angular rotation of a shaft (e.g., central shaft). Some layers on a shaft can have an angular rotation that is the same as the shaft while other layers have a different angular rotation. Angular rotation between a layer and a shaft can be proportional (e.g. 3:1 or 1:3).

Automated Device Control

Rotation of a device can be performed with the aid of a driving module (e.g., a base station) providing a source of mechanical force, such as a motor, a spring (e.g., a linear spring, a spiral spring, a torsional spring, a constant force spring), or an elastic band. Sources of mechanical force, such as motors, can be driven by power supplies. Examples of power supplies include but are not limited to batteries, solar panels, connectors or adaptors for wall or grid power, connectors or adaptors for motor vehicle power (e.g., 12 volt adaptor), hand cranks, and capacitors. When using a mechanical force, e.g. contained within a driving module, the device can comprise one or more flanges, knobs, slots or other structures adapted to engage with a non-rotating portion of the driving module to keep certain portions of the device stationary, thus allowing different parts of the device to rotate relative to one another.

The energy source for various manipulations, which may include slipping, pumping, and timing control, may be created, for example, by using a standard mechanical structure that can store potential energy in its deformed state. In one non-limiting embodiment, a constant-force spring may be used to provide energy and a constant force for achieving autonomous operations. In this embodiment, once the potential energy is stored in the deformed spring and the user initiates the controller, the stored potential energy will be released to form a mechanical force that controls the position of the architecture for driving the SlipChip to pump and slip (or relatively move the layers of a device) to effect reagent and fluid movement through the device.

In one embodiment, the driving module is provided in a base station. The devices or modules of the present disclosure can be used in conjunction with a base station to form an automated integrated device. In some examples, the coaxially arranged layers have minimal or no active mechanical or electronic components. When carrying out an assay, such mechanical or electronic functionality can be provided by a base station.

Primarily, a base station comprising a driving module can engage with the central shaft of a device and provide the motive force and control of rotation. Rotation of a device can be performed with the aid of a source of mechanical force, such as a motor, a spring (e.g., a linear spring, a spiral spring, a torsional spring, a constant force spring), or an elastic band. Sources of mechanical force, such as motors, can be driven by power supplies.

Constant Torque Spring/Motor

In some embodiments, provided herein is a device comprising a spring that can be used to actuate mobile parts in a device. In some embodiments, the device is an integrated device, a multicomponent device, or a SlipChip device. Springs that can be used include but are not limited to rotational springs, linear springs, constant force springs, and constant torque springs. In some embodiments, a constant torque spring can be used, in which the spring can be wound around one or more support cylinders (or drums). This configuration can provide constant torque, independent of the degree of rotation of the drums, and in some embodiments can be used to drive mobile parts and/or layers in a device.

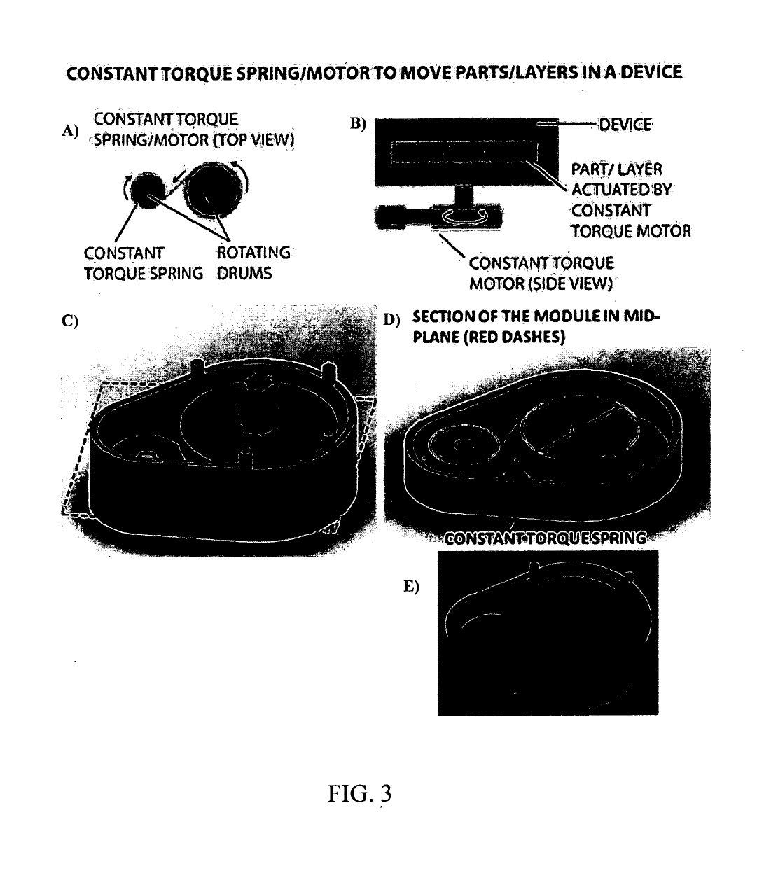

FIG. 3 depicts an embodiment of a constant torque spring used to move parts/layers in a device and/or provide pressure regulation. Panel A depicts a top view of schematics of the constant torque motor. Panel B depicts a side view of the interaction of the constant torque spring with the layers in the integrated device and its use to actuate mobile parts. Panel C depicts an embodiment of the enclosure of a constant torque spring motor, including tabs to attach the motor to another module to form an integrated device and a gear element to interact with and actuate a rotatable element in the attached module (e.g., a central threaded shaft) of an integrated device. Panel D shows a section of the exemplary torque spring motor along the mid plane (see dashed mid-plane in Panel C), showing the metal constant torque spring and the drums. Panel E shows a photo of an embodiment of a base station comprising a constant torque spring and drums, produced by 3D printing.

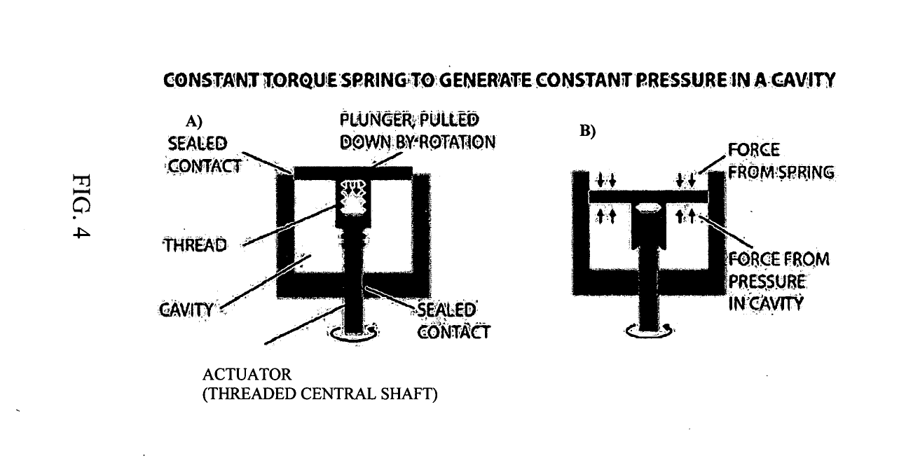

In some embodiments, a spring, e.g., a constant torque spring, can be used to generate constant pressure in a cavity of the device. In some embodiments the spring can be used to rotate a threaded component, connected to a separate part (such as for example a pressure cap) by the threads. In some embodiments, the pressure cap can be used to create a hermetic seal with a cavity. In some embodiments moving the pressure cap can change the volume in the cavity (FIG. 4). In some embodiments, the spring can be used to actuate the rotating part (actuator/threaded central shaft), thus moving the pressure cap and changing the pressure in the cavity. In some embodiments, the cavity may be hermetically sealed and rotation will stop when pressure reaches a maximum value, at which the force exerted on the pressure cap by the spring and by the pressure become equal. The actuator is actuated by a constant torque spring in this example. An example of this approach is described in panel B of FIG. 4.

In some embodiments, the spring will actuate in response to a pressure imbalance, such as for example in response to a pressure fluctuation in the cavity due to changes in temperature, volume, leaking from the sealing parts, or pumping of liquid. The maximum pressure can be tuned in a variety of ways, including but not limited to: changing the area of the pressure cap, the radius of the threaded region, the nature of the spring (torque) and the radius of the drum(s).

Without wishing to be bound by theory, it is believed that three main factors will influence the speed of rotation of a system similar to that shown in FIG. 4: the torque generated by the spring, the torque generated by friction of moving parts (generally modeled as proportional to the angular velocity) and the torque due to the pressure developed in the cavity (modeled as proportional to the rotation angle). Based on these assumption, the equations for predicting the behavior of a closed system similar to that described in FIG. 4 are:

$$3 TORQUES ACTING ON THE SYSTEM \\ 1 ) Spring torque ( T ) \to constant \\ 2 ) Friction coefficient ( F ) \to \\ {Torque }^{\sim} {F }^{*} angular velocity ( ω ) \\ 3 ) Torque due to pressure building up in cavity ( P ) \to \\ {Torque }^{\sim} {P }^{*} rotation angle ( θ )$$

$$Moment of inertia ( I )$$

$$Final equation \text{:} \begin{matrix}θ ( t ) = \frac{T}{P} ( 1 - {e}^{\frac{( F - \sqrt{4 A + {C}^{2}} ) t}{2 I}} ) \\ ω ( t ) = \frac{T}{P} \frac{( F - \sqrt{4 A + {C}^{2}} )}{2 I} \frac{( F - \sqrt{4 A + {C}^{2}} ) t}{2 I} .\end{matrix}$$

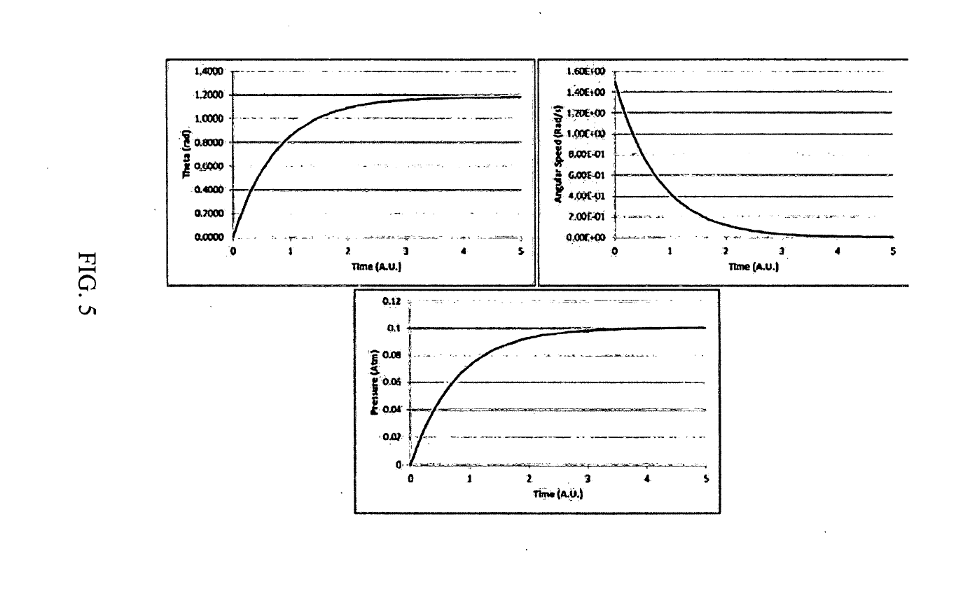

Graphs showing the expected trend for the pressure in this system over time according to the equations of the model is shown in FIG. 5. The graphs depict the expected angle of rotation (theta) (Panel A), angular speed (Panel B), and pressure in the cavity (Panel C) over time after release of the energy contained in the spring motor. When pressure reaches its equilibrium value with pressure cap force applied by the spring (0.1 atm in this case), the movement stops (angle is constant and angular speed approaches zero).

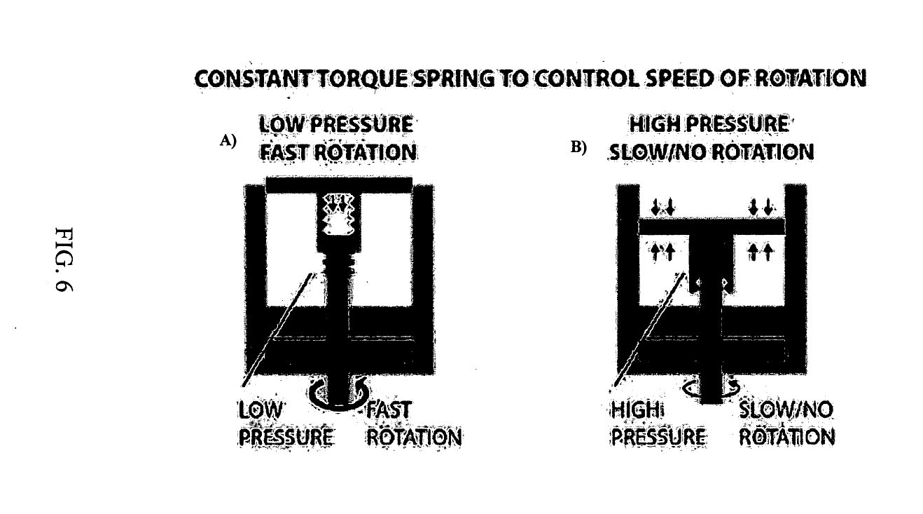

In some embodiments, the pressure build up in the cavity can be used to control the speed of rotation of another element in the device. For example, when low pressure is present, the torque created by the spring acting on the actuator (e.g., the central threaded shaft) can dominate and the parts can rotate at high speed FIG. 6. When the pressure builds up in the cavity (e.g. because the pressure cap is pulled down), the pressure can generate resistance to rotation and can slow down the moving parts (FIG. 6, Panel A). When the pressure reaches a maximum value equal to the force applied, rotation can slow or stop (FIG. 6, Panel B).

In some embodiments, the friction between moving parts can be controlled by modifying their dimensions, materials or relative positions. Higher friction will reduce the speed of movement and rotation. In some embodiments the friction is adjusted to regulate the operation of the device in combination with pressure changes. Since both friction and increased pressure decrease rotation speed, both may be optimized to control the rate of rotation for automation of the device.

Use of Pressure and Movement to Regulate Speed of Movement and Multi-Step Processes

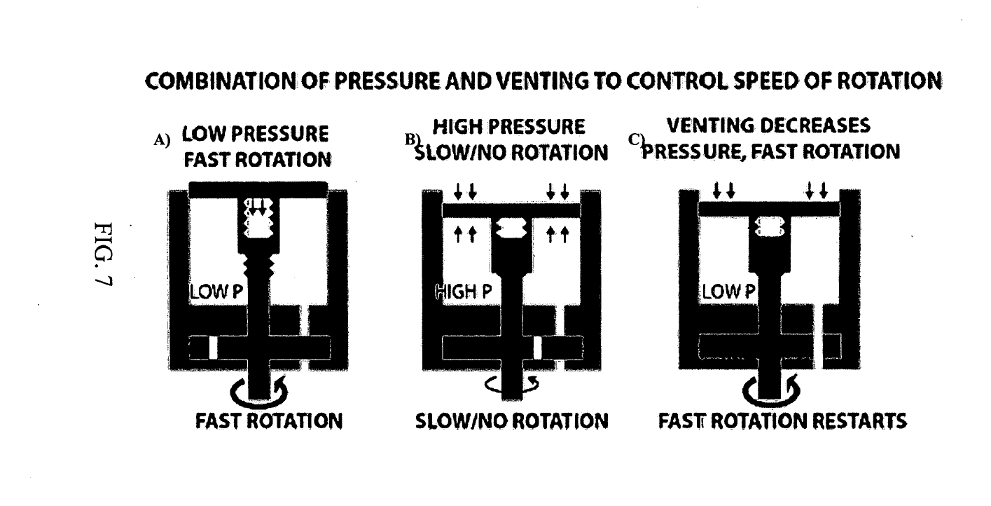

In some embodiments, the invention comprises a device (e.g., a microfluidic device, e.g., for two or more of sample preservation, sample storage, sample preparation, sample treatment, sample volume quantification, and/or sample analysis) that contains a spring, such as a constant torque spring, that can be used to control pressure in a cavity and the speed of movement of parts in devices, as described above. In some embodiments the pressure in the cavity can be reduced by creating reversible connections between the cavity and the external atmosphere. In some embodiments, when the connection is present, the air in the cavity is free to escape and the pressure is reduced. This situation can be referred to as “venting,” since the extra pressure in the cavity is released through a venting connection. An example of this strategy is shown in FIG. 7. In FIG. 7, a central threaded shaft is rotated by a driving module (e.g., a constant torque spring). The central threaded shaft is attached to a rotating layer. The rotating layer and the device both have via holes. When holes are not aligned, the system behaves as the one described in FIG. 6, where pressure increase decreases rotation speed. When the holes are aligned (FIG. 7, Panel C) the pressure in the cavity is released and the rotation speed of the central threaded shaft and attached layer will increase. In Panel A of FIG. 7, rotation of a central threaded shaft actuated by the motor (e.g., the spring motor) begins, the interface between the threads of the pressure cap and the threads of the central shaft pulling down the pressure cap to increase pressure. As pressure increases, the rotation of the central threaded shaft decreases (Panel B). Once a via hole in a rotating layer aligns with a via hole in the device, a vent is formed, and air is released from the chamber, decreasing pressure and allowing the rotation of the central shaft to increase in speed under the power of the driving module (Panel C).

In some embodiments of the device, this strategy can be used to pre-program the speed of rotation, such as for example by producing venting holes that align with the moving part in different positions: speed can be reduced by building up pressure, and then increased by inducing venting of the extra pressure in the cavity. In some embodiments, a multistep processes can be pre-programmed by the device design, and actuated by a single spring.

Use of Pressure and Liquid Pumping to Control Multi-Step Processes

In some embodiments, the system can be adapted to pump liquids through a device. In some embodiments, the devices are designed so that overlap of holes between two layers to form an open channel happens when the system has reached the maximum pressure (and/or minimum rotational speed), such as for example in the plateau condition described in FIG. 5.

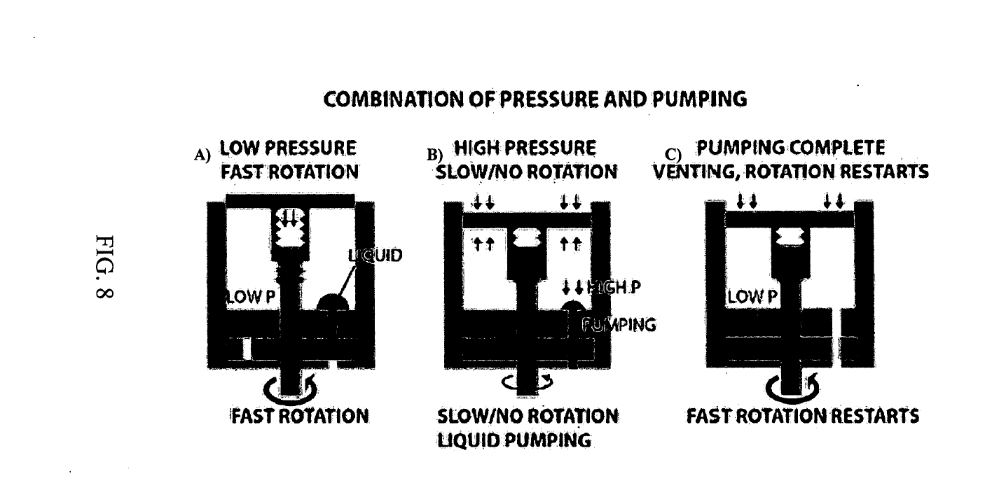

An example of the use of pumping to control multistep processes is described in FIG. 8. In this example, pressure will build up by rotation of the moveable part actuated by the constant torque spring (moving the pressure cap to increase pressure in the pressure chamber) until the overlap is reached. The liquid present in the cavity will be pumped out by the increased pressure present in the cavity, while the rotational speed is small or zero. After all the solution has been pushed out of the venting hole, the pressure is released and the system starts rotating at high speed again. In some embodiments, additional venting of air pressure will occur after all the liquid is pumped out to allow an increased angular velocity of the rotation of the layer.

In some embodiments, this approach can be used to control multistep delivery of liquid originally placed in a cavity. In some embodiments, liquids will be pumped separately and/or sequentially. For example, in some device embodiments, when the device is in the first overlap position, a solution will be pumped out of a first cavity and then the device will move into a second overlap position and pump a second solution. Devices may contain one or more overlaps and one or more solutions. The distance and/or angle among different overlap positions can be programmed by the device design.

In some embodiments the device is configured so that if no sample is present or insufficient sample is present at a specific location in the device, the device will continue to operate, such as by moving to the next step in the protocol.

In some embodiments, the device can be designed so that if a solution volume in one location is not pumped out completely in the time of overlap and venting is not possible, the rotation will stop between the two overlaps, and the protocol will not continue to completion.

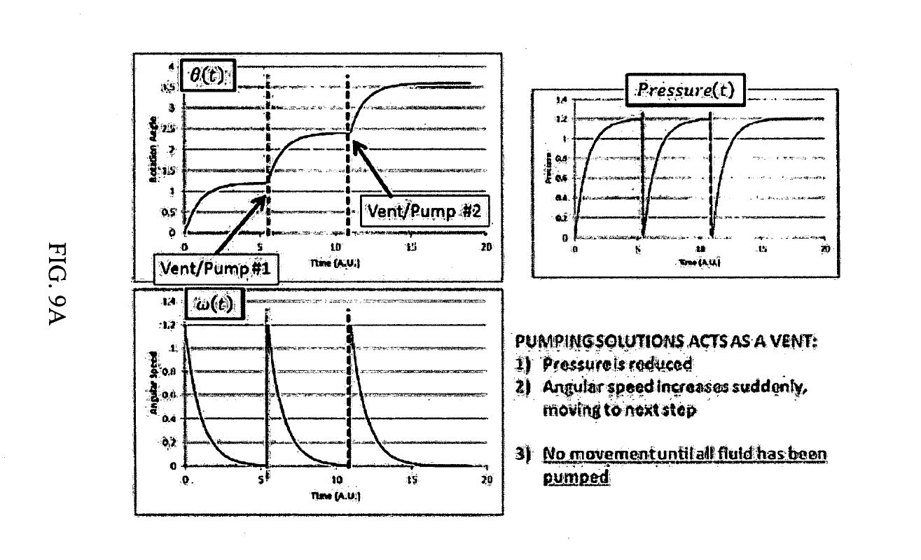

In some embodiments, the devices integrate multistep processes. FIG. 9A shows an example of a multistep process controlled by pumping and venting. The traces show the predicted value for the rotation angle (θ), rotation speed (μ(t)) and pressure in the cavity over time. Dashed lines each indicate the moment in which pumping and venting occurs at two sequential unique overlap position, calculated using the rotation model described above. Once the venting has released the pressure in the cavity, the layer starts rotating to the next position and building up pressure once again.

In an embodiment, the multistep process is the delivery of multiple solutions to a solid porous matrix, such as in the case of DNA and RNA purification protocols described in U.S. patent application Ser. No. 13/868,028. An example depicting the pumping of lysis buffer, wash buffer, and elution buffer to purify DNA or RNA from a sample through an extraction layer is depicted in FIG. 9B. The rotation angle versus time is reported in the graph. The shaded lines show the angle regions in which the holes overlap, and flow can be produced. This configuration allows up to three liquids to be pushed, but other embodiments can increase the number of total solutions pumped.

Combination of Pressure and Rotation to Generate Flow

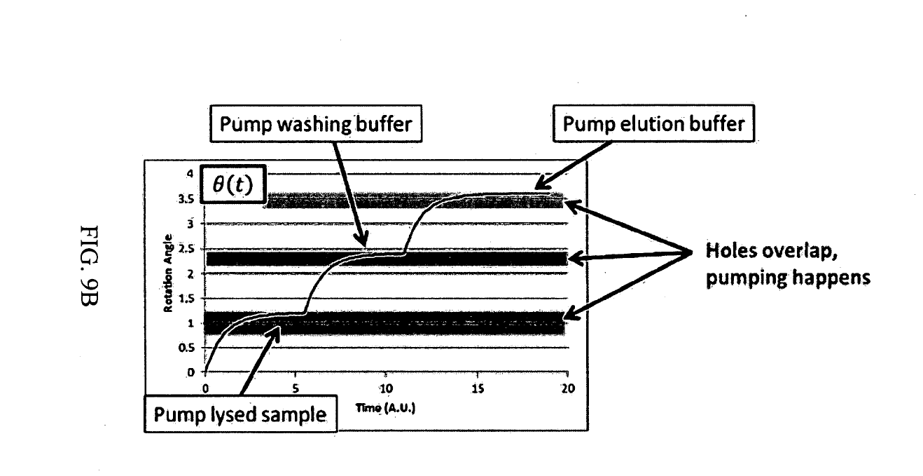

In some embodiments the pressure in the cavity can be used to generate liquid flow through the device, such as for example through a microchannel or other fluidic element and into an integrated or separate device. An example of this approach, showing the generation of liquid flow using the pressure produced by the constant torque approach is shown in FIG. 10. In Panel A, the system builds up pressure by moving the pressure cap and compressing the air in the cavity, while the hole in the rotating layer is moved towards the overlap position with a microchannel. Once the overlap position is reached, pumping is obtained by the pressure in the cavity, and is used to flow a liquid in a microchannel, a SlipChip, or other fluidic devices. The movable part is actuated by a constant torque spring in this example.

Mixing

In some embodiments the device uses flow and/or bubbles to mix multiple liquids. For example, the flow generated by the previous examples can be used to push one solution into another solution, and/or to push air into a liquid to create bubbles. In both cases mixing happens. This can be implemented in the devices described above by using two cavities containing liquids connected by a fluidic element (such as for example a channel or orifice), or by using a cavity/well and a blister.

In some embodiments the device uses flow and or bubbles to generate mixing. For example, the flow generated by the previous examples can be used to push a solution (such as a sample, a buffer or the eluted solution from a nucleic extraction module, etc.) in a compartment containing one or more compounds. Examples of these compounds can include reagents, enzymes, buffers, salts, nucleic acids, proteins, cells etc. in some embodiments these compounds are pre-stored in the device, in other embodiments they are placed in the compartment during device operation. These compounds can be in liquid state, or can be in solid state (e.g. dried) or other forms (such as in a gel, encapsulated in a sealed compartments etc.).

In some embodiments, the flow generated by the previous examples can be used to push a mixed solution to a different location in the device, such as for example in an amplification module for multiplex and/or quantitative (digital) detection. Primers for amplification may be present in the reagents compartment and/or pre-stored in the amplification module.

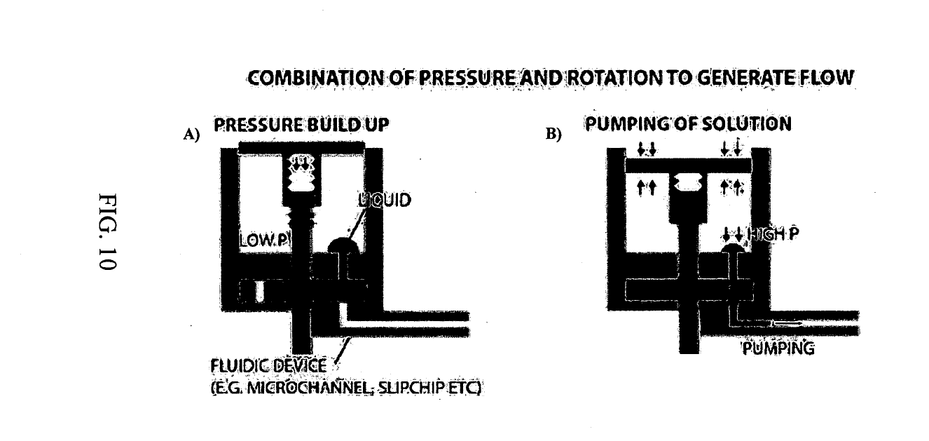

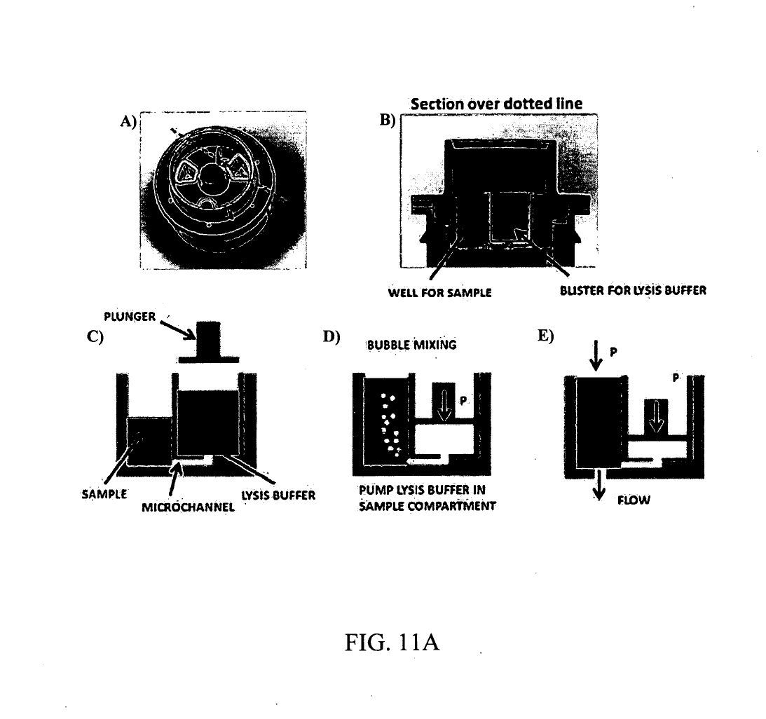

FIG. 11A shows an example of using the mixing process to mix a lysis buffer with a sample. Panels A and B show integration of reagent blisters in the extraction module described in U.S. patent application Ser. No. 13/868,028. Principles of the mixing operation are shown in Panels C-E. In Panel C, the two solutions are originally placed in two different compartments. The cavity may be used to place a first solution (“sample”) while the blister is used to store a second solution (“Lysis buffer”). These two solutions may be separated when the blister is intact, and may later be put in contact through a microchannel after piercing the blister. In Panel D, a pressure cap is used to push a second solution in the compartment containing the first solution to mix the two solutions. In some embodiments, after a solution has been pumped, air is pushed through the liquid, adding an extra step of mixing. Once the pressure is applied to both compartments, the mixed liquids can be pushed in a different location (Panel E) (e.g. through the venting hole for the multistep process described above). In some embodiments this approach can be used with multiple solutions, and multiple steps and it can also be integrated with an extraction module, such as for example the DNA/RNA extraction module described in U.S. patent application Ser. No. 13/868,028.

FIG. 11B shows an example of using the mixing process to mix a sample with amplification reagents. In Panel A, the sample and reagent are in separate compartments. In Panel B, a pressure cap is used to push the sample into the compartment containing amplification reagent to mix them. In Panel C, the mixed solution is then pushed to an amplification module for amplification of a target nucleic acid.

Layers

General Layer Characteristics

Each layer can have similar or different geometry compared to the other layers. A layer can be round, can have a plurality of straight edges, or can comprise a combination of arcs and straight edges.

Each layer can comprise at least one complete fluid passageway through the layer. Each layer can comprise one or more dead end paths that have an entry on the upstream side, but do not pass through the layer (e.g., a well). Wells can be used for applications such as holding waste products (e.g., lysed cell debris below filter, wash fluid) or receiving processed product material (e.g., purified nucleic acids).

Leakage between layers can be mitigated or prevented by methods including but not limited to the use of seals (e.g., elastomeric materials), sealing fluids (e.g., fluids immiscible with the fluids being flowed through the device), grease, soft waxes, and lubrication (e.g., dry lubricants, wet lubricants). For example, one or more layers can comprise an elastomeric material useful for creating a seal with adjacent layers.

In order to improve layer's sealing with adjacent layers, in some implementations, a deformable layer can be attached to both the top and bottom surfaces of the layer. Examples of deformable layer materials include but are not limited to silicone, polysiloxanes, polyurethane, rubber, chlorosulfonated polyethylene synthetic rubber, neoprene nylon, expanded polytetrafluoroethylene (PTFE), nitrile butadiene rubber (NBR), neoprene, nitrile nylon, and polydimethylsiloxanes. In an exemplary implementation, the deformable layer is silicone. The silicone, or other appropriate material, can deform under pressure and match the shape of the structures, e.g. entries and/or outlets, on the adjacent layers of the device, thus sealably engaging the present layer with adjacent layers. When a deformable layer is attached to the surface of a layer, sealing can be further enhanced by providing slightly raised ridges on the adjacent layer's surface. Preferably such ridges define an enclosed shape, typically surrounding an opening in that layer's surface. While such ridges can be any shape or height that would enhance sealing to the adjacent layer, the ridges can be about 0.05 μm, 0.075 μm, 0.1 μm, 0.11 μm, 0.12 μm, 0.13 μm, 0.14 μm, 0.15 μm. Inclusion of such raised ridges does not alter the complementary character of the surface of the layer.

Any layer in the device described herein can comprise one or more heating elements for altering or maintaining the temperature of fluid in one or more passageways through or wells in that layer. Said heating element can comprise, for example, Peltier elements or resistive heaters located in or adjacent to the layer. In other implementations, a foil heater can be wrapped around a passageway, such as a lysis well, to heat solution(s) contained therein. In other implementations, a heater can be placed inside the passageway.

After pressurization and forcing the sample into or through a particular passageway, excess air pressure can be released by allowing air to pass through one or more passageways. In this manner, an air flush can be used to remove dead volume in a matrix, chamber, conduit or other structure. In some embodiments, such an air flush can dry separation material in a separation layer. In some implementations, pressure within the device increases with each rotation providing additional force to move solutions through later-formed fluid paths.

In certain implementations, one or more layers are permanently affixed to other structures of the device. For example a layer engaged with the central shaft can be permanently affixed to (e.g. co-molded with) the threaded central shaft. See, for example, the separation layer 240 and central shaft 280 of FIGS. 2A and 2B. Similarly, a layer engaged with the housing can be permanently affixed to the housing. See, for example, the housing 210 and reagent layer 220 of FIGS. 2A and 2B. In other implementations, one or more layers can engage with the central shaft or housing in a detachable manner. For example, the layer can engage with one or more posts, knobs, keys/keyseats, grooves, gear teeth, slots, guiderails, or other features on the shaft or housing.

Wells, chambers, reagent packs, and other regions can be characterized by a volume. Such regions can have the same volume, or different regions can have different volumes. The volume of a well, chamber, reagent pack, or other region can be at least about 1 nanoliter (nL), 2 nL, 5 nL, 10 nL, 20 nL, 30 nL, 40 nL, 50 nL, 60 nL, 70 nL, 80 nL, 90 nL, 100 nL, 150 nL, 200 nL, 250 nL, 300 nL, 350 nL, 400 nL, 450 nL, 500 nL, 600 nL, 700 nL, 800 nL, 900 nL, 1 microliter (μL), 2 μL, 5 μL, 10 μL, 20 μL, 30 μL, 40 μL, 50 μL, 60 μL, 70 μL, 80 μL, 90 μL, 100 μL, 200 μL, 300 μL, 400 μL, 500 μL, 600 μL, 700 μL, 800 μL, 900 μL, 1 milliliter (mL), 2 mL, 3 mL, 4 mL, 5 mL, 6 mL, 7 mL, 8 mL, 9 mL, 10 mL, 15 mL, 20 mL, 25 mL, 30 mL, 35 mL, 40 mL, 45 mL, or 50 mL. The volume of a well, chamber, reagent pack, or other region can be at most about 1 nanoliter (nL), 2 nL, 5 nL, 10 nL, 20 nL, 30 nL, 40 nL, 50 nL, 60 nL, 70 nL, 80 nL, 90 nL, 100 nL, 150 nL, 200 nL, 250 nL, 300 nL, 350 nL, 400 nL, 450 nL, 500 nL, 600 nL, 700 nL, 800 nL, 900 nL, 1 microliter (μL), 2 μL, 5 μL, 10 μL, 20 μL, 30 μL, 40 μL, 50 μL, 60 μL, 70 μL, 80 μL, 90 μL, 100 μL, 200 μL, 300 μL, 400 μL, 500 μL, 600 μL, 700 μL, 800 μL, 900 μL, 1 milliliter (mL), 2 mL, 3 mL, 4 mL, 5 mL, 6 mL, 7 mL, 8 mL, 9 mL, 10 mL, 15 mL, 20 mL, 25 mL, 30 mL, 35 mL, 40 mL, 45 mL, or 50 mL. The volume of a well, chamber, reagent pack, or other region can be about 1 nanoliter (nL), 2 nL, 5 nL, 10 nL, 20 nL, 30 nL, 40 nL, 50 nL, 60 nL, 70 nL, 80 nL, 90 nL, 100 nL, 150 nL, 200 nL, 250 nL, 300 nL, 350 nL, 400 nL, 450 nL, 500 nL, 600 nL, 700 nL, 800 nL, 900 nL, 1 microliter (4), 2 μL, 5 μL, 10 μL, 20 μL, 30 μL, 40 μL, 50 μL, 60 μL, 70 μL, 80 μL, 90 μL, 100 μL, 200 μL, 300 μL, 400 μL, 500 μL, 600 μL, 700 μL, 800 μL, 900 μL, 1 milliliter (mL), 2 mL, 3 mL, 4 mL, 5 mL, 6 mL, 7 mL, 8 mL, 9 mL, 10 mL, 15 mL, 20 mL, 25 mL, 30 mL, 35 mL, 40 mL, 45 mL, or 50 mL.

Reagent Layer

The device described herein can comprise one or more reagent layers. The reagent layer can comprise one or more conduits or chambers capable of holding liquid or solid reagents for use during sample preparation. Reagents conduits or chambers can comprise simple chambers formed in the device layer, blister packs, or other containers.

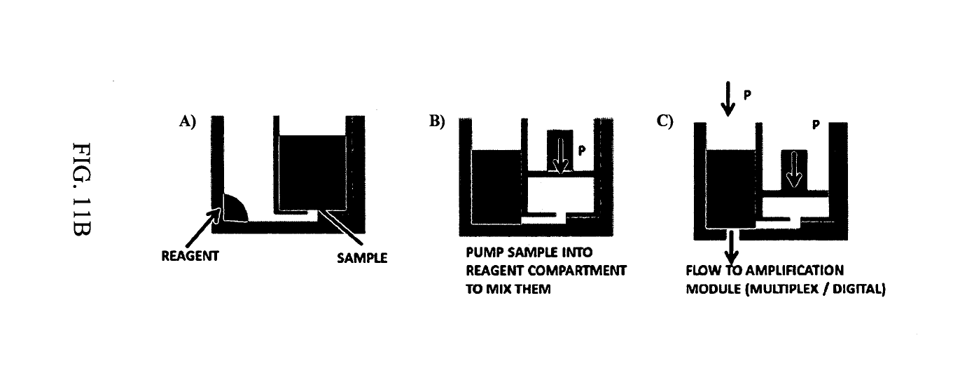

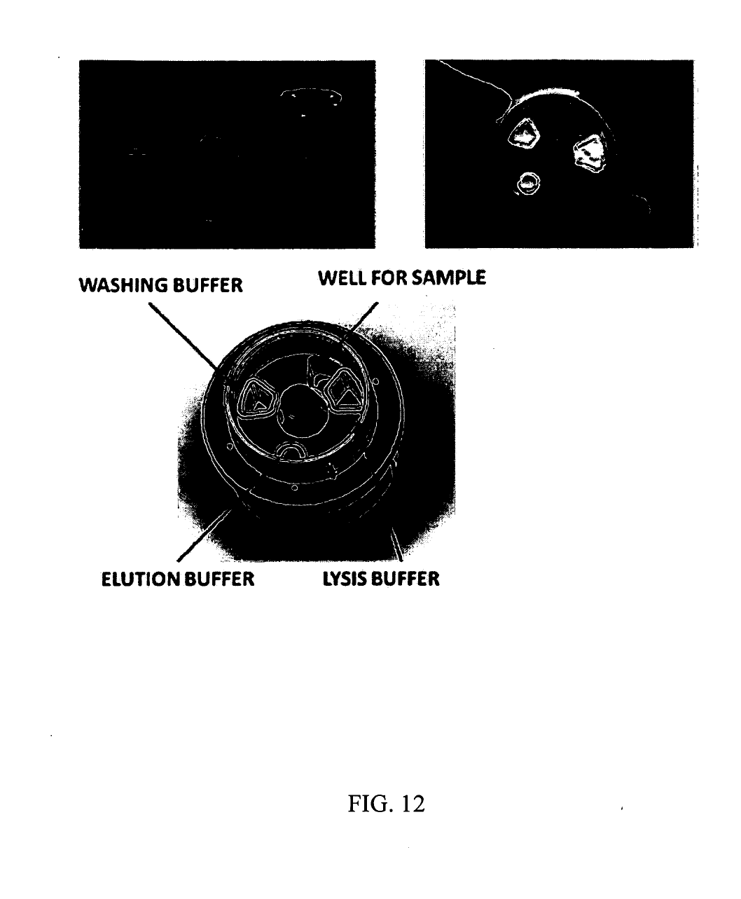

The device can comprise a blister pack layer. Such a blister pack can comprise blisters of a variety of sizes, for example from 1 μL to 10 mL. While any method known in the art can be utilized, preferably blisters are formed using heat-sealing, adhesive, pressure sealing, or other sealing mechanisms. In certain implementations, the layer comprising the blister pack has a piercing structure under the blister, and further the layer contains one or more passageways to dispense solution from blister to designed area after piecing. The piercing structure can be fabricated from metal, plastic, glass, or ceramics. Pressure built up with the device can be applied on the blister to cause shape deformation, and piercing structure can break the bottle seal of the blister, and solution can be dispensed through the fluidic path. Alternatively, the device can comprise a mechanism for physically breaking the blisters, such as a cam or posts projecting from an adjacent layer. An example of a blister pack for use in a reagent layer is depicted in FIG. 12. Panels A and B are photographs of blisters produced by 3D printing, and designed for integration with the reagent layer of the DNA/RNA extraction module describe in U.S. patent application Ser. No. 13/868,028, incorporated by reference herein in its entirety.

In some cases, the reagent layer includes a passageway adapted for receiving a sample and lysing cells, i.e. a lysis chamber. The lysis chamber may have any suitable shape and configuration, but typically will be in the form of a well or chamber of sufficient volume to receive and process a clinically relevant sample. The lysis chamber may be adapted for lyses of eukaryotic or prokaryotic cells as well as disruption of certain particles in a fluid sample.

In one implementation, the reagent layer receives the sample in a first fluid path that is open to the upstream and downstream surfaces. The reagent layer can comprise a second fluid path that is open to the upstream and downstream surfaces

In certain examples, the device comprises a plurality of reagent storage layers. For example, the module can comprise a first reagent storage layer holding reagents related to nucleic acid isolation and a second reagent storage layer holding reagents related to nucleic acid amplification. In such an example, the first reagent storage layer would typically be placed upstream of a separation layer and the second reagent storage layer would be placed downstream of the separation layer.

In certain implementations, the reagent layer can comprise a passageway arranged as a fragmentation unit. In some methods, random fragmentation of DNA or RNA can be desirable, or even necessary, as a sample pre-treatment step. Fragmentation can be achieved biochemically using restriction enzymes, or through application of a physical force to break the molecules. The positive pressure generated by the pressure cap during operation of the devices described herein can be used to fragment nucleic acid as the sample passes through a short constriction (e.g., by shear or other stress). In some implementations, DNA and/or RNA breaks under mechanical force when pushed through a narrow orifice, due to rapid stretching of the molecule. A pressure-driven flow can lead to a shear force, which leads to fragmentation of the nucleic acids.

Reagents can be preloaded on the device. Reagents also can be loaded by a user. In some cases, some reagents are provided preloaded on the device and some (e.g., perishable reagents) are provided by a user prior to operation. Reagents can be provided in wet or dry form. In some examples, the reagent storage layer is preloaded with one or more reagents. In such an example, the reagents can be contained with a membrane configured to be pierced or disrupted during operation of the module. In some examples, the membrane comprises foil, laminate and/or plastic. In other examples, dry reagents are rehydrated by a user prior to use of the device. For example, a user can load water into a device to rehydrate reagents, and then a user can load a sample into the device and operate the device.

Exemplary reagents can include, but are not limited to, lysis solutions, wash solutions, elution solutions, rehydration solutions, enzyme solutions (e.g., nucleic acid amplification enzymes, polymerase enzymes, restriction enzymes), buffers, liquid, powder, pellets, a gel, microbeads, probes, primers, nucleic acids, DNA, RNA, polypeptides, nucleoside triphosphates (NTPs), antibodies, a sacrificial reagent or any combination thereof. A sacrificial reagent can comprise an aqueous solution, a lubricant, an oil, an aqueous-immiscible liquid, a gel, a gas, a fluorocarbon oil, a surfactant, gas, air, or any combination thereof. For example, the air can be used to generate air bubble for mixing. As another example, air and immiscible liquid can be used to remove leftover solution (dead volume) in the matrix. Reagents can be mixed to change their composition. For example, one type of buffer can be mixed with another buffer or a dry reagent to change its composition to another buffer.

A device can include at least 1, 2, 3, 4, 5, 6, 7, 8, 9, 10, 15, 20, 25, 30, 35, 40, 45, 50, or more different reagents. A device can include volumes of reagents including at least about at least about 1 nanoliter (nL), 2 nL, 5 nL, 10 nL, 20 nL, 30 nL, 40 nL, 50 nL, 60 nL, 70 nL, 80 nL, 90 nL, 100 nL, 150 nL, 200 nL, 250 nL, 300 nL, 350 nL, 400 nL, 450 nL, 500 nL, 600 nL, 700 nL, 800 nL, 900 nL, 1 microliter (μL), 2 μL, 5 μL, 10 μL, 20 μL, 30 μL, 40 μL, 50 μL, 60 μL, 70 μL, 80 μL, 90 μL, 100 μL, 200 μL, 300 μL, 400 μIL, 500 μL, 600 μL, 700 μL, 800 μL, 900 μL, 1 milliliter (mL), 2 mL, 3 mL, 4 mL, 5 mL, 6 mL, 7 mL, 8 mL, 9 mL, 10 mL, 15 mL, 20 mL, 25 mL, 30 mL, 35 mL, 40 mL, 45 mL, or 50 mL each.

Separation Layer

The device described herein can comprise one or more separation layers. The separation layer can comprise one or more passageways occluded with a separation material, said separation material capable of binding an analyte of interest, such as a molecule or cell.

Systems and devices described herein can comprise filters, membranes, gels, and other separation materials, which can be located on a separation layer. The presence of a separation material in a channel, open chamber, or conduit can increase the resistance to flow through that structure. Parameters of a separation material can be chosen to provide a specific resistance to control the operation time and pressure required to pass a sample or reagent into or through the separation material. Parameters of a separation material can include thickness, density, porosity, pore size, wettability or hydrophobicity, binding affinity, or electrical charge of the material. For example, a separation material with greater thickness or smaller pore size can be characterized by a larger resistance to flow and can increase the amount of time or pressure required for a fluid or reagent to dispense from a prior layer. On the other hand, a separation material with lesser thickness or larger pore size can be characterized by a smaller resistance to flow and can decrease the amount of time for a fluid or reagent to dispense from a prior layer.

The separation material can be any useful material for binding one or more molecules of interest. The separation material can be any useful material for binding one or more other analytes of interest (e.g., cells, spores, particles). Exemplary materials includes a filter, a matrix, a polymer, a charge switch material, a gel, and a membrane (e.g., a silica membrane, a glass-fiber, membrane, a cellulose membrane, a nitrocellulose membrane, a polysulfone membrane, a nylon membrane, a polyvinylidene difluoride membrane, a vinyl copolymer membrane, or an ion exchange membrane, including any described herein a fiber (e.g., a glass fiber), or a particle (e.g., a silica particle, a bead, an affinity resin, or an ion exchange resin). In certain implementations, the separation material can comprise a capture moiety. Suitable capture moieties include small organic molecules, such as dyes and tryazines, and biopolymers such as peptides, proteins (including antibodies, and fragments thereof), polynucleotides, oligosaccharides or lipids. Capture moieties of the present invention may be molecules having molecular weights of 100 KDa or more, such as antibodies, but preferably are smaller molecules with a molecular weight in the range of 10 KDa, more preferably around 1 KDa, desirably less than 1 KDa for example, less than 750, 500, or 250 Da. Ideally, capture moieties are coupled to an insoluble particulate or polymeric material. Each insoluble particle preferably carries several copies of the same capture moiety, with each particle type coupling a different capture moiety. Separation materials, such as membranes or filters, can be characterized by a pore size or an average pore size of at least about 0.1 μm, 0.2 μm, 0.3 μm, 0.4 μm, 0.5 μm, 0.6 μm, 0.7 μm, 0.8 μm, 0.9 μm, 1.0 μm, 1.1 μm, 1.2 μm, 1.3 μm, 1.4 μm, 1.5 μm, 1.6 μm, 1.7 μm, 1.8 μm, 1.9 μm, 2.0 μm, 2.1 μm, 2.2 μm, 2.3 μm, 2.4 μm, 2.5 μm, 2.6 μm, 2.7 μm, 2.8 μm, 2.9 μm, 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, 5.0 μm, about 10 μm, about 15 μm, about 20 μm, or about 25 μm. In some cases, the pore size or average pore size of a separation material is from about 0.7 μm to about 4.0 μm.

Separation materials can have a thickness of about 0.1 μm, 0.5 μm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 mm, 2 mm, 3 mm, 4 mm, 5 mm, 6 mm, 7 mm, 8 mm, 9 mm, 10 mm, or more. Separation materials can have an area of about 1, 2, 5, 10, 20, 50, 100, 200, 500, 1000, 2000, 5000, 10000, 20000, 50000, or 100,000 square micrometers or 1, 2, 3, 4, 5, 6, 7, 8, 9, 10, 20, 30, 40, 50, 60, 70, 80, 90, or 100 square millimeters, or more.

Receiving Layer

A receiving layer can comprise one or more chambers or wells to receive fluids. For example, a receiving layer can comprise one or more product wells to receive a product, such as purified nucleic acids. A receiving layer can comprise one or more waste wells to receive waste fluids, such as wash buffers. A waste well can comprise one or more absorbents. Absorbents can be used to absorb fluids such as waste fluids. Exemplary absorbents include but are not limited to pads, diaper polymers (such as polyacrylic acid variants), powders, particles, pellets, gels, paper, fabric, fibers, capillary and desiccants.

Conduits, chambers, wells, and other structures on a receiving layer can be open- or closed-ended. Open-ended conduits can allow transit of fluids of interest to subsequent layer, such as an analytic layer. At least a portion of the material or substrate defining a passageway, e.g. a conduit, chamber, well, or other structure, can be transparent, translucent, or otherwise compatible with taking measurements of the sample inside (e.g., optical measurements). In some cases, an entire layer can be transparent or translucent. In some implementations, particularly those in which detection is performed by a base station, the layer most proximal to the drive shaft is partially or fully transparent.

Chambers or wells in a receiving layer can comprise or connect to outlets allowing the recovery of material. For example, purified nucleic acids can be recovered for subsequent use off-device.

Analytic Layer

The modules, devices and systems described herein can be integrated with other devices to allow multistep processes. For example, the sample preparation modules can be included in a device by exploiting the modularity of SlipChip devices, in order to prepare the sample before storage. Examples include but are not limited to devices for multistep protocols for nucleic acid extraction and filtration elements to separate plasma from whole blood using membranes and/or integrated filtration elements such as geometrical features in the device (for example, restrictions or a gap between the layers). In a preferred implementation, the analytic layer comprises a microfluidic device.

An analytic layer can comprise chambers or other features for analysis of a sample, such as by nucleic acid amplification. An analytic layer can comprise reagents for analysis, such as nucleic acid reagents; alternatively, an analytic layer can receive analysis reagents from a reagent layer.

Analysis can indicate the presence, absence, or quantity of an analyte of interest. For example, nucleic acid amplification can provide qualitative or quantitative information about a sample, such as the presence, absence, or abundance of a cell, cell type, pathogen (e.g., bacteria, virus), toxin, pollutant, infectious agent, gene, gene expression product, methylation product, genetic mutation, or biomarker (e.g., nucleic acid, protein, or small molecule).

Analytical targets of interest can include indicators of diseases or illnesses such as genetic diseases, respiratory diseases, cardiovascular diseases, cancers, neurological diseases, autoimmune diseases, pulmonary diseases, reproductive diseases, fetal diseases, Alzheimer's disease, bovine spongiform encephalopathy (Mad Cow disease), chlamydia, cholera, cryptosporidiosis, dengue, giardia, gonorrhea, human immunodeficiency virus (HIV), hepatitis (e.g., A, B, or C), herpes (e.g., oral or genital), human papilloma virus (HPV), influenza, Japanese encephalitis, malaria, measles, meningitis, methicillin-resistant Staphylococcus aureus (MRSA), Middle East Respiratory Syndrome (MERS), onchocerciasis, pneumonia, rotavirus, schistosomiasis, shigella, strep throat, syphilis, tuberculosis, trichomonas, typhoid, and yellow fever. Analytical targets can include biomarkers indicative of traumatic brain injury, kidney disease, cardiovascular disease, cardiovascular events (e.g., heart attack, stroke), or susceptibility of certain infectious agents (such as bacteria or viruses) to certain therapeutic agents. Analytical targets can include genetic markers, such as polymorphisms (e.g., single nucleotide polymorphisms (SNPs), copy number variations), gene expression products, specific proteins or modifications (e.g. glycosylation or other post-translational processing) of proteins.

Passageways

Layers can comprise one or more passageways. A passageway can be a channel, conduit, chamber, or other structure in which at least one complete path traverses the layer. The passageways can include any useful cross-section or plurality of cross-sections along their paths. Cross-sections can be of any useful shape (e.g., rectangular, square, circular, oval, trapezoidal, triangular, or irregular cross-sections). Cross-section shape or dimensions can vary along the axis of any structure. For instance, the cross-section of the passageway along the axis of fluid flow can change from one cross-sectional shape or area to another, such as from a circular to a rectangular cross-section. In another instance, the dimensions of the cross-section can be uniform or can vary along any axis, such as a passageway that tapers or expands along the axis of fluid flow.

Similarly, the path of any passageway can be linear, twisting, curved, serpentine, or any other track shape. Twisting or serpentine paths may be selected to encourage mixing of components of a fluid. The passageways can additionally contain columns, posts, dimples, humps, weirs, hydrophobic patches, hydrophilic patches, or other structures to improve mixing of fluids as they pass. Implementations in which the passageway is linear can achieve rapid transfer of a fluid under minimal pressure. The passageway can be substantially axially aligned, with the upstream opening being directly or nearly directly above the downstream opening. Alternatively, the upstream and downstream opening can be offset by any distance.

A passageway or channel can have a cross-sectional area of at least about 1, 2, 5, 10, 20, 50, 100, 200, 500, 1000, 2000, 5000, 10000, 20000, 50000, or 100,000 square micrometers, or 1, 10, 20, 30, 40, 50, 60, 70, 80, 90, 100, 200, 300, 400, 500, 600, 700, 800, 900 or 1000 square millimeters. A passage or channel can have a cross-sectional area of at most about 1, 2, 5, 10, 20, 50, 100, 200, 500, 1000, 2000, 5000, 10000, 20000, 50000, or 100,000 square micrometers or 1, 2, 3, 4, 5, 6, 7, 8, 9, 10, 20, 30, 40, 50, 60, 70, 80, 90, or 100 square millimeters. A passage or channel can have a cross-sectional area of about 1, 2, 5, 10, 20, 50, 100, 200, 500, 1000, 2000, 5000, 10000, 20000, 50000, or 100,000 square micrometers or 1, 2, 3, 4, 5, 6, 7, 8, 9, 10, 20, 30, 40, 50, 60, 70, 80, 90, or 100 square millimeters.

For passageways, the size, length, cross-sectional area, other geometric factors, or any combination thereof can be selected to control flow rates, pressures, or other characteristics of fluid flow through the passageway.

Layers can comprise additional passageways (e.g., vents) for pressure balance or equalization rather than liquid handling. Such passageways can be distributed around the edge or circumference of the layer.

Each layer can include at least one passageway that opens to both the upstream and downstream surfaces of the layer. Additionally, the openings can encompass channels or apertures arranged on the surface that form conduits and/or chambers when mated to adjacent layer(s). These channels can be formed by a variety of means, such as by being molded or cut into the layers. Alternatively, the openings in the surfaces can be defined by a gasket material between the layers.

Modules and Integration

In some embodiments, the device comprises one or more modules for sample processing or analysis. In some embodiments, the processing steps in each module are automated by rotation of one or more layers in the device. In some embodiments, the rotation of one or more layers in the device is actuated by a torque spring or other motor integrated with the device.

Sample Preparation Module

In some embodiments, the device comprises a sample preparation module. In some embodiments, samples can be split into different compartments that may or may not contain a drug of interest (such as an antibiotic). In some embodiments, after incubation of the sample with/without antibiotic drug, the cells of interest (such as for example those of a bacterial pathogen) are lysed (such as by lysis buffer) in order to extract RNA and/or DNA. In some embodiments, the sample-preparation module can be used to automatically extract nucleic acids from urine. In some embodiments, for sample preparation, a filter column can be used to capture RNA/DNA from a lysed sample and wash away cell debris or other components present in the lysed sample.

In some embodiments, an automated or semi-automated sample preparation module is provided. In some embodiments, the automated or semi-automated sample preparation module generates pressure to the lysed sample in a sealed environment, forcing the lysed sample through a filter column (e.g., a nucleic acid-binding column, such as a glass fiber column).

In some embodiments, the sample prep module is designed to process between 1 mL and 10 mL. In some embodiments, the sample prep module is designed to process between 100 μL and 100 mL. In some embodiments, the sample prep module is designed to process between 10 μl and 1 L. In some embodiments, the sample prep module is designed to process between 1 μL and 1 mL. In some embodiments, the sample preparation module can be made of plastic and in some embodiments does not require external power or active user intervention.

In some embodiments, the sample preparation module comprises a reagent layer, a separation layer comprising a membrane (e.g., a capture layer), and a receiving layer. The reagent layer can comprise a lysis buffer chamber, a wash buffer chamber, and an elution buffer chamber. The reagent layer can also comprise a chamber for sample. In some embodiments, the receiving layer comprises one or more receiving chambers. The reagent layer can include more than one chamber for the washing buffer and/or elution buffers to effect more than one washing and/or eluting steps. At least one layer can be moved to connect a sample chamber, a capture region, and a receiving well along a single path. Pressure can then be applied to transport the sample through the capture region and into the aligned receiving well, where the desired analyte is captured in the capture region. In some embodiments, at least one layer is then moved to a second position to connect the capture region, a wash buffer chamber, and a receiving chamber. Pressure is applied to transport wash buffer through the capture region, thereby performing the washing step. In some embodiments, at least one layer is then moved to a third position to connect a capture region, an elution chamber, and a receiving well. Pressure is applied to transport the elution buffer through the capture region, thereby eluting the molecule of interest into the receiving layer. Movement of the at least one layer can include any useful movement that moves the layers relative to the chamber or capture region including the sample and/or analyte.

Amplification Module

In some embodiments the device can be used for multiplex amplification, such as nucleic acid amplification and detection. In some embodiments the device can be produced by 3D printing or other fabrication methods, using a variety of plastic and non-plastic materials.

In some embodiments the device design includes two layers, including wells and ducts for fluid handling (FIG. 13). The layers can be produced using a rigid material (such as e.g. Veroclear, from Stratasys) and can include a thin layer (˜100-200 microns) of soft material (such as e.g. TangoPlus, from Stratasys) in the contact surface. A lubricant can be used to seal the gap and allow relative movements of the layers. In some embodiments these devices can be integrated with an extraction module, such as for example the DNA/RNA extraction module described in U.S. patent application Ser. No. 13/868,028. In some embodiments modules of the device can be clamped, such as for example using pins and hex fits, in order to maintain the parts in tight contact.

In some embodiments layers in the amplification module can be operated in multiple positions, and/or can have different alignments between the layers. An embodiment of a two layer device for multiplex nucleic acid amplification is shown in FIG. 13. The loading position is used for loading the solutions in the wells. In this embodiment, sample is injected through an inlet, and fills six wells (each 10 microliters in volume). Buffer is injected in a separate series of wells for negative controls. Extra wells (labeled as “thermal expansion wells”) are present in the device, and can be pre-filled with amplification reagents and primers. These may be provided in liquid or dried (e.g., lyophilized) from. Each of these extra wells can be filled with different sets of primers for multiplex amplification.

In some embodiments, one or more layers of the amplification module are rotated to place the amplification module in the amplification position (FIG. 13). In the amplification position, each sample/buffer well is separated from the others so that amplification can be performed in individual wells without cross-contamination. The sample/buffer wells contact a well comprising reaction mix and primers to facilitate the amplification reaction in each well. This enables different reactions to be performed in the same device, and using a single inlet for the sample. Amplification reactions that can be used in some embodiments include PCR, LAMP, RPA, NASBA etc.

In some embodiments, when the device is in the amplification position, each well containing the loaded sample is placed in contact with one or more additional wells, in a predetermined way; the entities contained in these wells are then able to mix and the reaction is initiated. In some embodiments of the device multiplex amplification can be obtained by loading different primers in different additional wells, so that each sample well is exposed to a different set of primers. FIG. 13 shows an example with three different additional wells, each labeled with a different color, wherein each well can be used to load a different reagent (such as for example an amplification mix, primers, probes etc.) for multiplex reactions.

In some embodiments the device can include lyophilized reagents, such as amplification reagents for LAMP detection. The reagents stored in the device can also include indicators for visual readout, that can be lyophilized together with the other reagents or dried independently. In some embodiments the stored reagents can be included in stabilizing matrices (e.g. trehalose) or in other forms, such as the Biospheres commercialized by Biolyph Corporation.

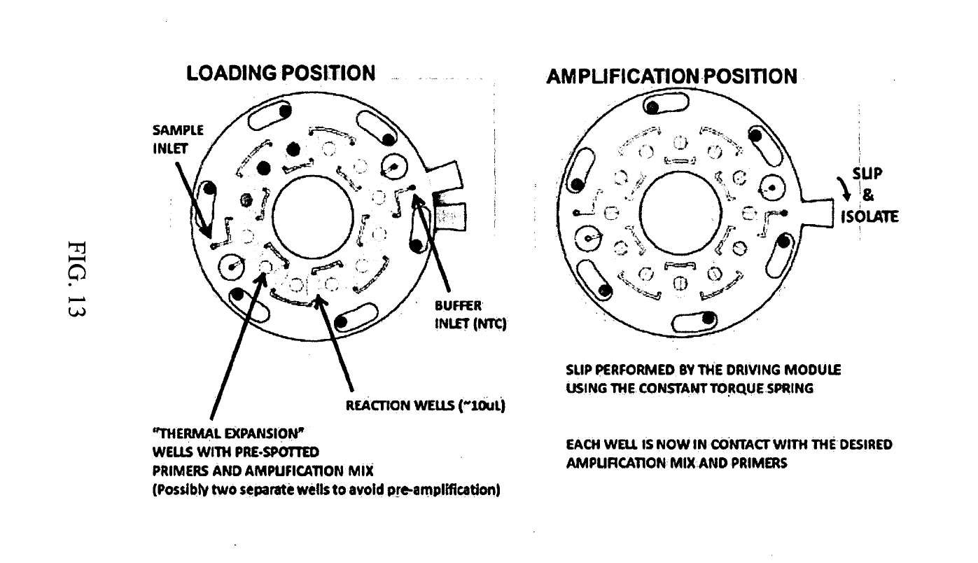

FIG. 14 shows the loading of two solutions in a device made with 3D printing for multiplex amplification. Each “thermal expansion” well is pre-filled with Fe(III) solution, while the sample wells are filled with two different aqueous solutions containing KSCN and a food dye (green or yellow). Upon slipping, all wells enter in contact with the Fe(III) solution, and thus turn red due to the presence of Fe(SCN)3 (iron dye).

In some embodiments the amplification module can be used for detection, such as for example quantitative detection of analytes (such as nucleic acids, proteins, cells, molecules, and others). In some embodiments, the amplification module is a digital (or binary) quantification modules, such as modules containing multiple detection elements in which 0, 1, or more target (e.g. RNA/DNA) molecules are captured per each detection element and are suitable for integration.

Visual readouts can also be used in some embodiments of the device, such as systems based on alpha naphtol (Colorimetric detection of loop-mediated isothermal amplification reaction by using hydroxy naphthol blue, Motoki Goto, Eiichi Honda, Atsuo Ogura, Akio Nomoto, and Ken-Ichi Hanaki, D.V.M. BioTechniques, Vol. 46, No. 3, March 2009, pp. 167-172).

In some embodiments, enriched target (e.g. DNA/RNA) are recovered from the device for use in subsequent or parallel experiments. In some embodiments, the integrated device has a plurality of quantification modules.

In some embodiments, the amplification module is designed to process between 30 μL, and 60 μL. In some embodiments, the amplification module is designed to process between 1 μL and 100 μL. In some embodiments, the amplification module is designed to process between 100 nL and 1 mL. In some embodiments, the amplification module is designed to process between 10 nL and 10 mL.

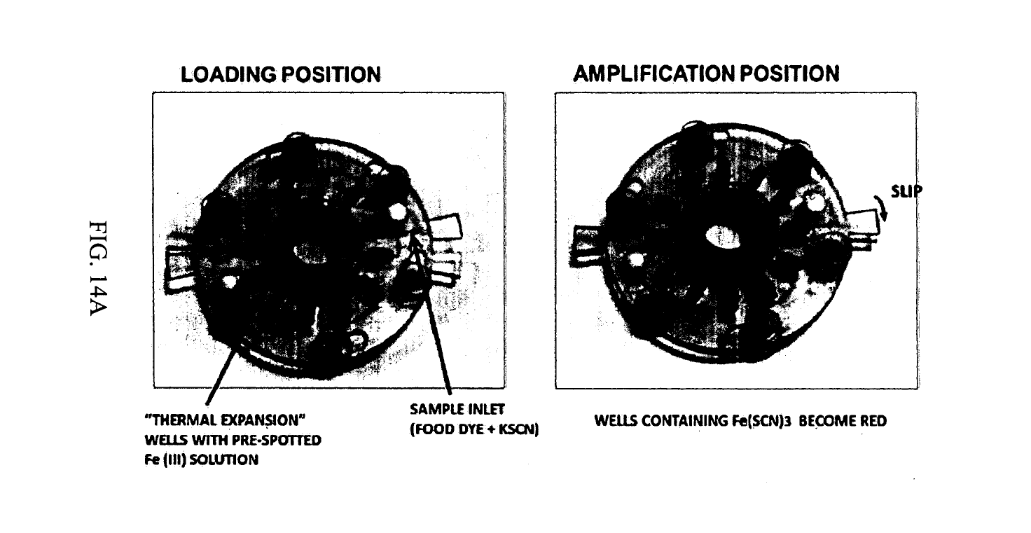

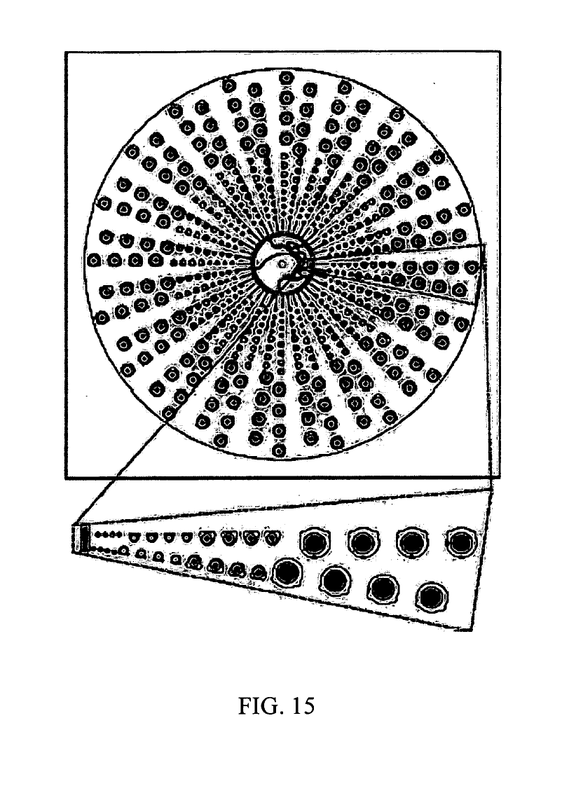

In some embodiments, an amplification module for digital single molecule detection can be integrated with other modules, including an extraction module, such as for example the DNA/RNA extraction module described in U.S. patent application Ser. No. 13/868,028, or any of the other features described in this document (including but not limited to actuators based on springs, reagent storage containers, heating modules, etc.). The integration of these modules can enable digital and quantitative autonomous measurements. FIG. 15 shows an embodiment of a design of an amplification module for digital/single module detection and/or analyte quantification.

In some embodiments, the amplification module can include multiple sets of wells to be added to the reagents at different times, enabling the performance of multistep reactions in the device. For example, a sample inserted in the amplification module can be split into a set of aliquots (wells) and combined with a first set of wells containing one or more amplification reagents. Another aliquot can be combined with a second set of wells, containing one or more amplification reagents. Multiple sets of wells can be added, allowing the addition of three, four or more reagents to each well.

In some embodiments, this multi-step approach can be used to inject the eluted sample from a nucleic acid purification module, then mix it with amplification reagents (such as enzymes, buffers, primers etc.) to perform the amplification reaction (e.g. a multiplex amplification reaction). After the amplification reaction has completed, the wells can be put into contact with one or more reagents (e.g. detection reagents) present in another set of wells. The amplification module thus enables multistep reactions to be performed therein.

The rotation of layers within the amplification module can be performed by the automated device and/or by the user when detaching the disposable device from its base station.

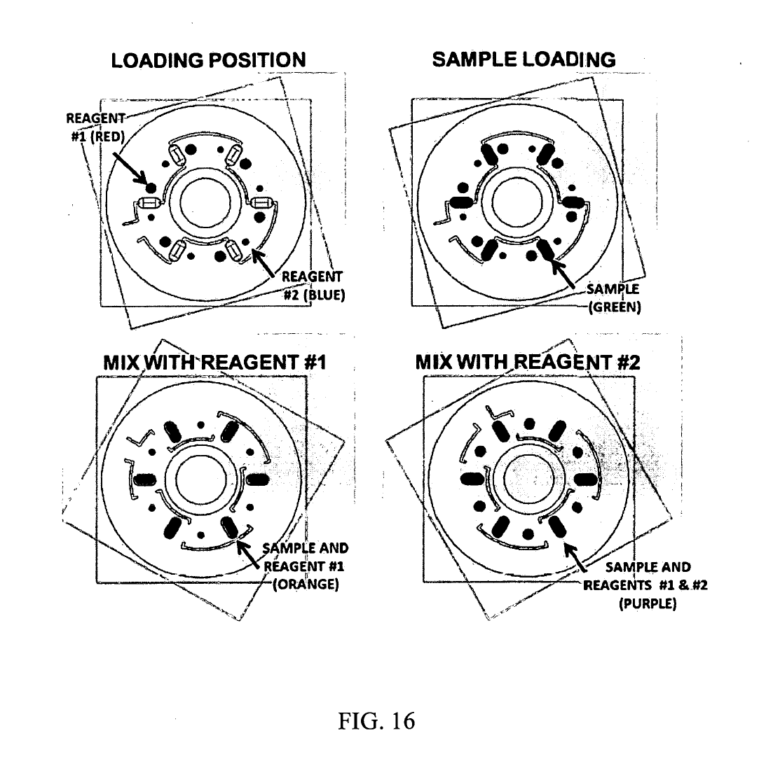

Shown in FIG. 16 is an example of a two-step reaction performed in an amplification module. First, the device is provided in the loading position. Reagent #1 and Reagent #2 are contained in separate sets of wells in a first layer of the module. Sample is then loaded into the device into wells in a second layer separate from the reagent wells in the first layer. Next, a layer in the amplification module is rotated so that the sample wells of the second layer and the wells of the first layer containing Reagent #1 overlap, thereby combining each aliquot of sample Reagent #1. Finally, a layer in the amplification module is again rotated so that the sample wells of the second layer overlap with the wells of the first layer containing Reagent #2, thereby resulting in the combination of sample, Reagent #1, and Reagent #2.

Base Station/Driving Module

The devices of the present disclosure can be used in conjunction with a base station. In some examples, the coaxially arranged layers have minimal or no active mechanical or electronic components. When carrying out an assay, such mechanical or electronic functionality can be provided by a driving module. In one embodiment, the driving module is a base station.

Primarily, a driving module can engage with the central shaft of a device and provide the motive force and control of rotation. Rotation of a device can be performed with the aid of a source of mechanical force, such as a motor, a spring (e.g., a linear spring, a spiral spring, a torsional spring, a constant force spring), or an elastic band. Sources of mechanical force, such as motors, can be driven by power supplies. Examples of power supplies include but are not limited to batteries, solar panels, connectors or adaptors for wall or grid power, connectors or adaptors for motor vehicle power (e.g., 12 volt adaptor), hand cranks, and capacitors.

In some embodiments, the driving module comprises a spring to provide torque to the central shaft. In some embodiments, the spring is a rotational spring, linear spring, constant force spring, or constant torque spring. FIG. 3 shoes an embodiment of a driving module comprising a constant torque spring wound around one or more support cylinders (e.g., drums). This configuration can provide constant torque, independent of the degree of rotation of the drums, and in some embodiments can be used to drive mobile parts and/or layers in a device.

Heating Module

In some embodiments the device comprises an integrated heating module (i.e., a heating element). In some embodiments, heating modules are embedded in the device, such as those into which an incubation module can be inserted, such as for example a bath containing high thermal conductivity materials. In some embodiments, amplification and detection of a target (e.g. RNA/DNA) can include heating elements on the device. Examples of heating elements can include, but are not limited to, heating by using electrical power, chemical reaction, or phase change materials. In some embodiments, transparent thermally conductive materials can be used such that images of the amplification region can be captured through the heating unit.

Heat from the heating module can be obtained, for example by electrical power, e.g., using resistors and a voltage or current source (such as a battery, a voltage generator etc.). In some embodiments the heating process can be activated by external triggers, such as for example a reversible connector actuated by mobile parts in an integrated device.

In some embodiments energy generated by the heating module can be used to heat the desired parts directly, or it can be used in combination with a phase change material (PCM). Below melting temperature (Tm), the PCM is solid. When heat is transferred to the PCM, its temperature rises up to the Tm, and stays at Tm until all the material is melted (similarly to an ice bath), thus maintaining a defined temperature. In some embodiments, a device combining an electrical heater with a PCM can be used to heat a system to the desired temperature for a desired time.

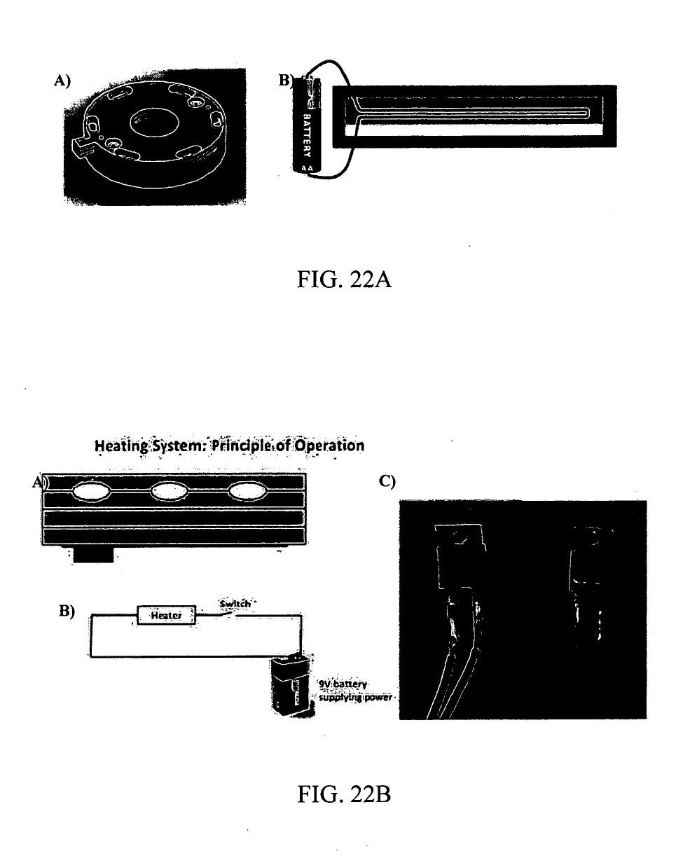

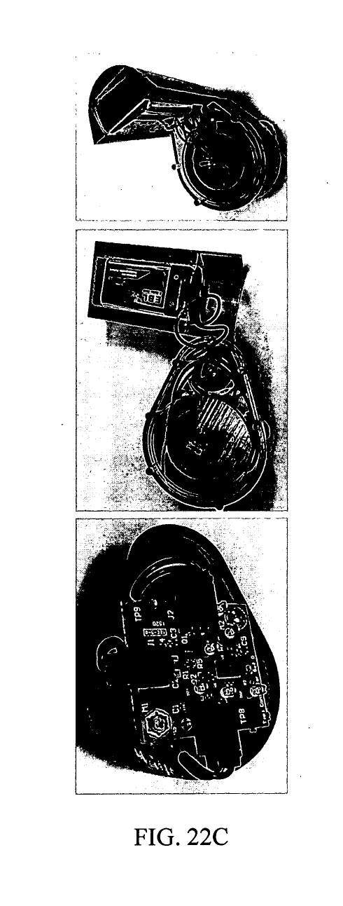

In one embodiment, paraffin wax with melting temperature of 65° C. can be used to bring a system to that temperature, and this temperature can be used in combination with some embodiments of the device to run a reaction, such as an amplification reactions (e.g. LAMP). FIG. 22A shows an example of heating module in which a resistor is embedded in paraffin wax. This heating module can be used to heat an amplification module. The tight contact between the two modules can be obtained by using clamps, pins with hex-fits or any other clamping mechanism. As shown in Panel A, the heater (bottom) is placed in contact with an amplification module (top). As shown in Panel B, this embodiment of the heating module comprises a resistor (black line attached to battery) attached to a power source, where the resistor is placed inside a phase change material (e.g., paraffin wax) in the heating module. When the circuit is closed the heater starts melting the PCM, bringing the system to a defined temperature (e.g., 65° C. for paraffin wax).

In some embodiments a desired temperature can be achieved by using a sensing element to control the heating circuit. Example of sensing elements include thermostats, thermal switches, thermistors and other thermal sensors. These thermal sensing units can be used to control the heater, limiting the heating action by reducing the current or switching the current on and off depending on the temperature at the sensing unit.

As an example, mechanical thermostats can be used to produce the circuit. FIG. 22B shows an embodiment of a heating circuit with a sensing element and switch for assays requiring temperature control. In an embodiment, a heater and switch are attached to an aluminum block and the amplification module is placed on top of the aluminum, as shown in Panel A. An embodiment of an electrical circuit for use with a heating element, including a 9V battery, a 10 Ohm heater and a mechanical thermal switch is shown in Panel B. Photos of an embodiment of the thermostat are shown in Panel C. This circuit was used to perform the LAMP amplification in the amplification modules described above.

A heating module with a sensing element as depicted in FIG. 22B can be successfully used to generate a temperature of 63° C. in a device, and was used to perform LAMP amplification in the amplification modules described above. This heating module was integrated with an amplification module to amplify DNA (lambda), viral RNA (HCV) and bacterial 16S RNA (Chlamydia trachomatis and Neisseria gonorrhoeae) as detected both with fluorescence and visual readout (e.g., hydroxynaphthol blue and Eriochrome Black T).

In one embodiment the heating module comprises integrated circuit boards comprising a switching circuit with a comparator and transistors to switch the heating element on and off depending on the temperature detected by a thermistor. In an embodiment, the integrated circuit board is connected to the heating element that is heated using a Nichrome wire. In some embodiments, the heating module controlled by the integrated circuit board is capable of heating up the amplification module to 63+/−2° C. within 2 min. FIG. 22C illustrates how the circuit board is integrated in an autonomous device, according to an embodiment of the heating module described above. In one embodiment, the circuit board is mounted underneath the constant torque spring housing (i.e., the driving module). In one embodiment, a battery attached to the circuit board is placed in a dedicated holder that next to the DNA/RNA extraction module. FIG. 22C shows photographs of an embodiment of the heating module comprising an integrated circuit board and battery for heating up a heater for performing nucleic acid amplification.

Integration of Modules

In some embodiments, the devices provided herein are integrated devices comprising one or more modules. These modules include, but are not limited to, an incubation module, a sample preparation module, an amplification module, and a readout module. In some embodiments, the integrated device combines 2 or more modules to provide a simplified processing flow for performing an assay. The modules and devices described in this document can be integrated to obtain a complete device, capable of performing biological assays, even if they require multiple steps.

In some embodiments the device can be used for an assay such as for example sample processing (e.g. lysis of raw biospecimen), analyte purification, analyte amplification and detection, sample mixing, sample aliquoting, sample drying, heating, and more. Analytes that can be treated include and are not limited to proteins, nucleic acids, microorganisms such as bacteria, viruses etc., small molecules, metabolites and others. Samples include and are not limited to urine samples, blood samples, blood plasma samples, serum samples, sputum samples, sweat samples, culture media, buffers, water samples, environmental samples, etc.

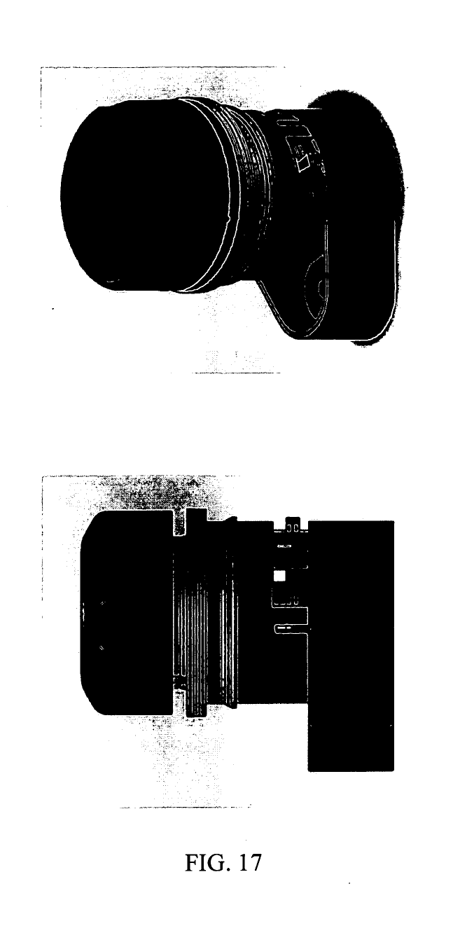

In some embodiments device integration can be obtained by producing multiple modules in a single part (using injection molding, 3D printing, or other fabrication techniques) or connecting different modules present in different parts. In some embodiments of the device, connection of different modules can be obtained by complementary features (including but not limited to luer locks, screw mechanisms etc.), or by keeping the parts in close contact (by using clamps or other similar mechanisms). Examples of integrated devices that can be used for detection of nucleic acids in a sample (e.g., for multiplex detection of Chlamydia and Gonorrhea) are shown in FIGS. 17, 18, 19 and 20. FIG. 17 shows an example of an integrated device that can be used to detect nucleic acid in urine samples. FIG. 18 shows a cross-sectional view of an embodiment of an integrated device comprising a pumping lid, blisters for reagent storage, a nucleic acid extraction module, an amplification module, a heating module, and a driving module.

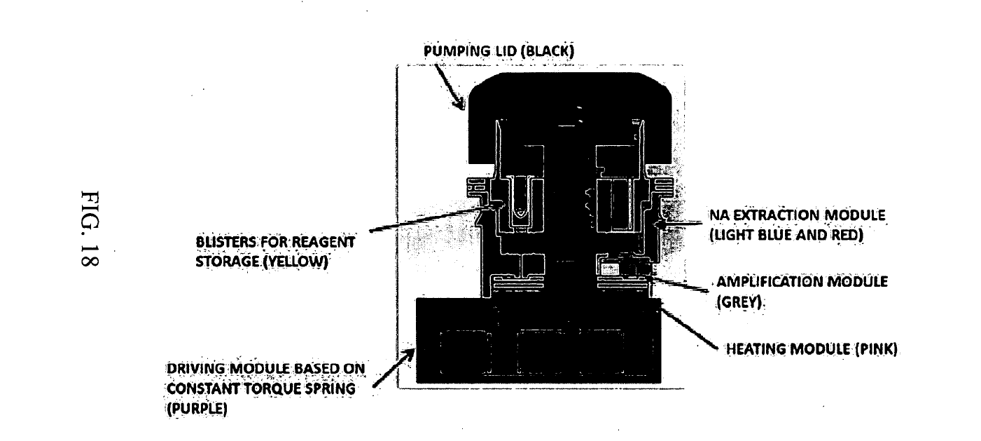

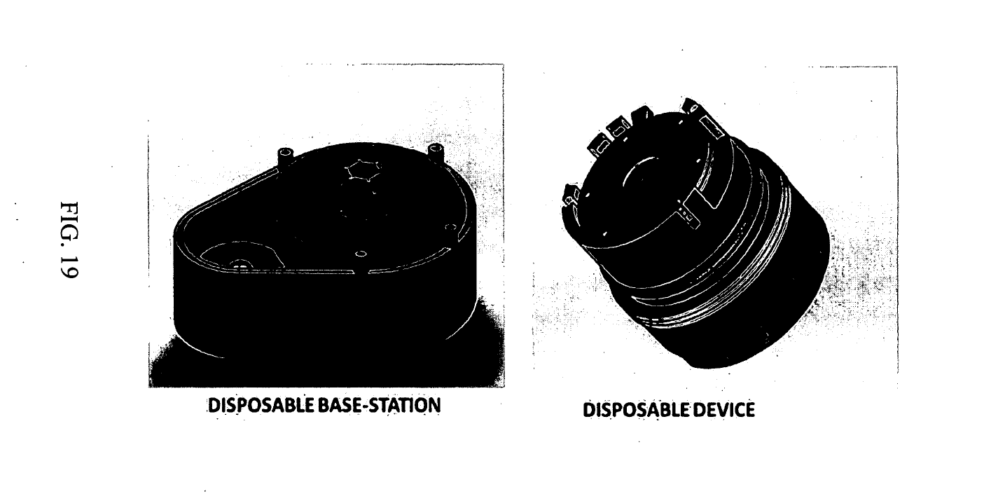

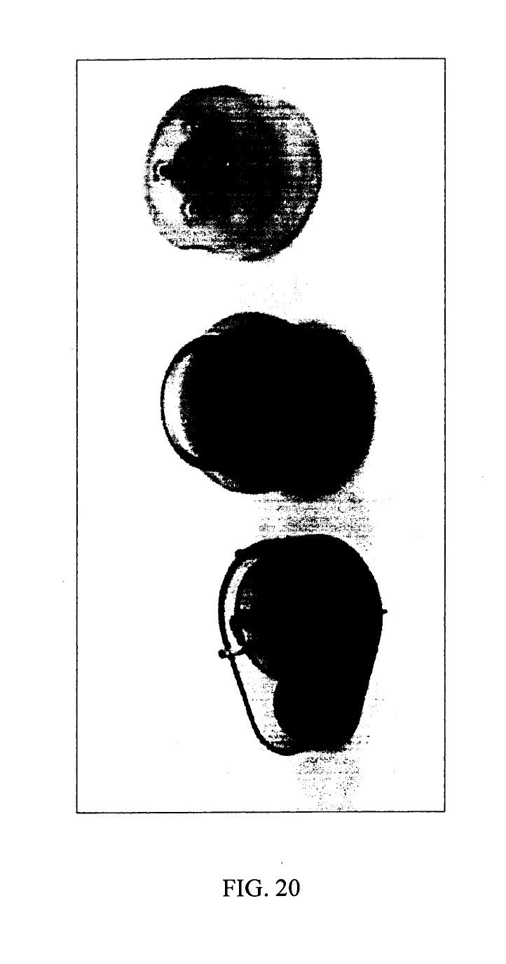

In some embodiments, some parts of the integrated device can be detached after use. For example, the sample preparation and amplification module can be removed from the rest of the device. FIG. 19 shows a disassembled integrated device after use, exposing the amplification module. Images can be taken of the amplified solutions in the amplification module to detect presence of a nucleic acid target in the starting sample. FIG. 20 shows a photograph of modules of an embodiment of the device fabricated with 3D printing and having the same geometry as the embodiments depicted in FIGS. 17, 18, and 19.

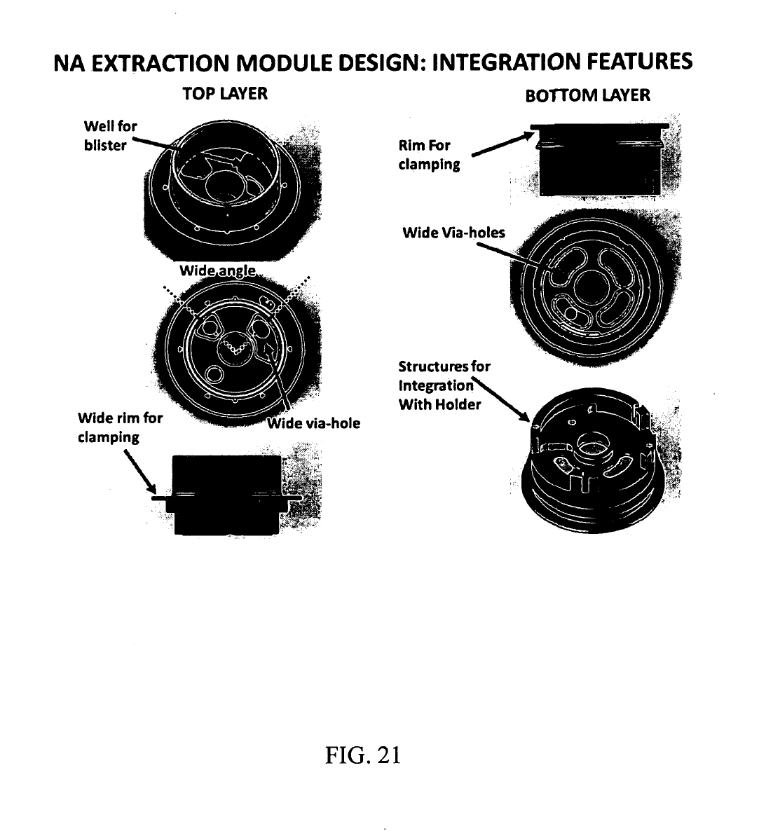

In some embodiments, the nucleic extraction module can include features to enable integration and automation, such as such as: well geometries that accommodate blisters, wide via holes and wide angles between via holes, rims for clamping and structures for integration with the driving module/holder. Examples of these features are shown in FIG. 21.

In some embodiments, the detachment of one or more parts can be used as an actuation mechanism to perform operations on one or more modules. For example, the removal of one module can trigger slipping or other mechanisms in the device, by actuating parts while the module is removed. For example, this removal can include twisting or other movements that can be used for actuation.

In some embodiments, connections between modules consists of a soft material, which can be, but is not limited to, silicone, polydimethylsiloxane or TangoPlus (from Stratasys). To ensure successful fluid loading and enable accurate fluid metering, air is evacuated through a hydrophobic, porous membrane, such as for example, but not limited to, PTFE membrane (for example, 0.45 μm pore size) while blocking aqueous solution when it contacts the membrane.

Formation of Module

Modules and/or devices can be formed by any useful process, including but not limited to molding (e.g., injection molding, vacuum molding, or over-molding), machining (e.g., drilling, milling, or sanding), and etching (e.g., deep reactive ion etching, KOH etching, or HF etching). In microfluidic applications, the layers can be fabricated from a material that enables formation of high resolution features (e.g., microchannels, chambers, mixing features, and the like, that are of millimeter, micron, or submicron dimensions), such as by using microfabrication techniques (e.g., dry etching, wet etching, laser etching, laser ablation, molding, embossing, or the like, to have desired miniaturized surface features). Further, the material can be optionally treated to provide a chemically inert surface (e.g., by silanization with tridecafluoro-1,1,2,2-tetrahydrooctyl-1-trichlorosilane), a biocompatible surface (e.g., by treatment with bovine serum albumin), and/or a physically stable material (e.g., by extensive cross-linking). In some embodiments, modules or devices can be fabricated by a number of methods including 3D printing, injection molding, embossing, wet etching, and other methods known in the art.

Materials

The substrates or layers can include any useful material. For instance, a portion of a layer can include a membrane, or the entire layer can include a continuous membrane or a patterned membrane. Furthermore, such membranes can be integrated with one or more layers (e.g., by over-molding or lamination) having one or more chambers and/or inlets. Alternatively, such membranes can be present in a separate layer. Exemplary membranes include a PTFE (e.g., Teflon®) membrane, a polycarbonate membrane, a cellulose membrane, a nitrocellulose membrane, a nylon membrane, a paper membrane, or other membranes that are known in the art.

In some embodiments, the device comprises flexible materials such as, but not limited to PDMS, Tango+ (a 3D-printable soft material), or rubber. The flexible materials enable the device to perform leak-proof liquid movement of a sample among modules (e.g., between the sample preparation module to the amplification module). In some embodiments, the device comprises channels/microchannels to interface modules. In some embodiments, the device comprises syringe pumps/teflon tubing. In some embodiments, the device comprises embedded channels in 3D-printed parts. In some embodiments, the device comprises 3D-printed valves. These components can also enable the device to perform leak-proof liquid movement of a sample among modules.

In some embodiments, the device can also include one or more deformable layers. Such deformable layers can be designed to deform as pressure is applied, such as to redistribute local pressure into uniform pressure over a surface of the device and/or to control connection or disconnection between layers or chambers.

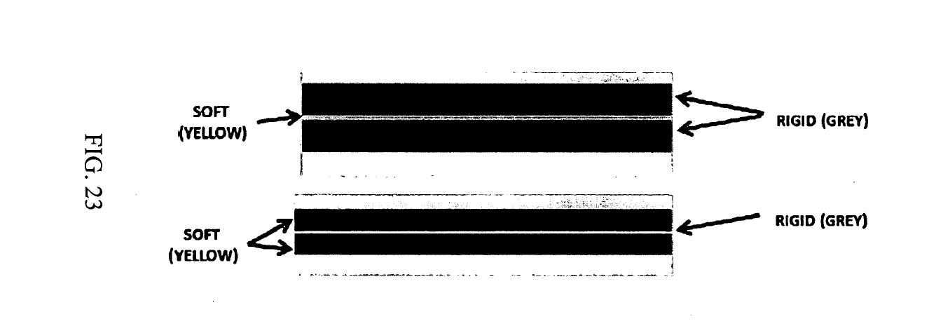

In some embodiments a device or module can be produced using materials showing different properties, such as elastic module, color, chemical composition, thicknesses etc. For example, devices can be made of two materials, showing different mechanical properties: one material being rigid (e.g. veroclear from Stratasys) and one material being soft, or rubber like (e.g. tango plus or tango black plus from Stratasys). Using a combination of soft and rigid material enables the presence of compliant and rigid regions in the device. This can be used to improve the clamping in a SlipChip device. If there is any imperfection in the device (e.g. due to manufacturing strategy), this can be adsorbed by the compliant layer and the performances of the device will not be affected. The compliant (soft) layer can be used in proximity of the gap and/or in the bulk of the devices. An example of a series of layers in a module or device having compliant layers in proximity of the gap and in the bulk of the device is shown in FIG. 23, showing soft material in proximity of the gap, while the majority of the device is made of rigid material (grey).

In some embodiments, the device comprises polymeric materials, such as silicone polymers (e.g., polydimethylsiloxane and epoxy polymers), polyimides (e.g., commercially available Kapton® (poly(4,4′-oxydiphenylene-pyromellitimide, from DuPont, Wilmington, Del.) and Upilex™ (poly(biphenyl tetracarboxylic dianhydride), from Ube Industries, Ltd., Japan)), polycarbonates, polyesters, polyamides, polyethers, polyurethanes, polyfluorocarbons, fluorinated polymers (e.g., polyvinylfluoride, polyvinylidene fluoride, polytetrafluoroethylene, polychlorotrifluoroethylene, perfluoroalkoxy polymer, fluorinated ethylene-propylene, polyethylenetetrafluoroethylene, polyethylenechlorotrifluoroethylene, perfluoropolyether, perfluorosulfonic acid, perfluoropolyoxetane, FFPM/FFKM (perfluorinated elastomer [perfluoroelastomer]), FPM/FKM (fluorocarbon [chlorotrifluoroethylenevinylidene fluoride]), as well as copolymers thereof), polyetheretherketones (PEEK), polystyrenes, poly(acrylonitrile-butadiene-styrene)(ABS), acrylate and acrylic acid polymers such as polymethyl methacrylate, and other substituted and unsubstituted polyolefins (e.g, cycloolefin polymer, polypropylene, polybutylene, polyethylene (PE, e.g., cross-linked PE, high-density PE, medium-density PE, linear low-density PE, low-density PE, or ultra-high-molecular-weight PE), polymethylpentene, polybutene-1, polyisobutylene, ethylene propylene rubber, ethylene propylene diene monomer (M-class) rubber), and copolymers thereof (e.g., cycloolefin copolymer); ceramics, such as aluminum oxide, silicon oxide, zirconium oxide, and the like); semiconductors, such as silicon, gallium arsenide, and the like; glass; metals; as well as coated combinations, composites (e.g., a block composite, e.g., an A-B-A block composite, an A-B-C block composite, or the like, of any materials described herein), and laminates (e.g., a composite material formed from several different bonded layers of identical or different materials, such as polymer laminate or polymer-metal laminates, e.g., polymer coated with copper, a ceramic-in-metal or a polymer-in-metal composite) thereof.

Coating

One or more substrates, one or more layers, and/or chambers can be optionally coated. In particular cases, a coating can be used to minimize cross-contamination between layers, where relative movement between layers can result in thin films of reagents forming between layers. The coating can be used to control surface chemistry (e.g., by increasing the contact angle to about 154° with water). In particular cases, one or more layers and/or chambers are coated with a fluoropolymer. Exemplary fluoropolymers include fluorinated ethylene propylene resin (e.g., Teflon® FEP TE-9568, a dispersion composed of approximately 54% (by total weight) of a negatively charged, hydrophobic colloidal fluoropolymer resin (0.1 rto 0.30*m(m FEP particles suspended in water) and approximately 6% (by weight of FEP resin) of a nonionic wetting agent and stabilizer based on the weight of the FEP solids), perfluoroalkoxy copolymer resin (e.g., Teflon® PFA TE-7224, a dispersion composed of approximately 60% (by total weight) of PFA resin (0.05 to 0.5 μm particles) dispersed in water and approximately 5% by weight of a nonionic wetting agent and stabilizer based on the weight of the PFA solids; or Teflon® PFAD 335D, a dispersion composed of approximately 60% (by total weight) of PFA resin (0.20 μm average diameter particles) dispersed in water and approximately 6% by weight of a nonionic surfactant based on the weight of the PFA solids), polytetrafluoroethylene (e.g., Teflon® PTFE DISP 30, a dispersion composed of approximately composed of approximately 60% (by total weight) of PFTE resin (0.220 μm average diameter particles) dispersed in water and approximately 6% by weight of a nonionic surfactant based on the weight of the PTFE solids), or a copolymer of tetrafluoroethylene and ethylene (e.g., Tefzel® Type LZ, CLZ, or CLZ-20, available in nominal gauges of 50, 100, 200, 500, 750, 1000, or 2000, having a thickness of 0.0005, 0.0010, 0.0020, 0.0050, 0.0075, 0.0100, or 0.0200 inches).

The systems, devices, and methods can include any useful lubricant. In some examples, the lubricant is used as a sacrificial fluid (e.g., as described herein), facilitates movement of the first, second, and/or intermediate substrates or layers, and/or minimizes contamination between the first, second, and/or intermediate layers or chambers within these layers or substrates.

In addition, the lubricant can be selected to be substantially inert with respect to the substances (e.g., reagents and/or samples) that will be in contact with and/or transported through the device. For instance, the lubricant can optionally be a fluid that is substantially immiscible with the reagent(s) and/or sample(s). The lubricant can optionally be selected to have physical characteristics that promote compartmentalization of the reagent(s) and/or sample(s). For instance, the layers and/or chambers can be fluorophilic, and the lubricant can be a fluorous liquid. In this example, compartmentalization occurs by competing surface characteristics, where surface tension results in separating reagent and/or sample fluids into separate plugs or droplets encapsulated by the lubricant.

Exemplary lubricants include a hydrocarbon, a fluorous substance, an ionic liquid, a non-Newtonian fluid, or a lubricating powder or bead. Exemplary hydrocarbons include alkanes, paraffin oils, hexane, hexadecane, silicon oil, greases (e.g., Dow Corning high vacuum grease, Fomblin vacuum grease, Krytox greases), mineral oil, and other organic materials or polymers, as well as mixtures thereof. Exemplary fluorous substances include fluorocarbons (including perfluorinated and semifluorinated alkanes, e.g., octadecafluoro-decahydronaphthalene and perfluorooctylethane), alkyl and aryl fluorocarbons, halofluorocarbons (e.g., perfluorooctyl bromide), fluorinated alcohols (e.g., 1-(1,2,2,3,3,4,4,5,5,6,6-undeca-fluorocyclohexyl)ethanol or C₆F₁₁C₂H₄OH), fluorinated oils, liquid fluoropolymers (e.g., perfluoropolyethers), Fluorinert (3M), Krytox oils, Fomblin oils, and Demnum oils.

Ionic liquids include a cation and an anion, which form a salt and are in a liquid state. Exemplary cations include choline; imidazolium-based cations, such as optionally substituted imidazolium-based cations (e.g., 1-C1-10 alkyl-3-C1-10 alkyl-imidazolium, (3-C1-10 alkyl-imidazolium-1-yl)-C1-10 alkanol, or 1-C₁₋₁₀alkyl-2,3-di-C₁₋₁₀ alkyl-imidazolium, such as 1-C₁₋₁₀ alkyl-3-methyl-imidazolium, (3-methylimidazolium-1-yl)-C₁₋₁₀alkanol, or 1-C₁₋₁₀ alkyl-2,3-dimethylimidazolium) or bicyclic imidazolium-based cations (e.g., optionally substituted 2,3-(CH₂)₂₋₆-imidazolium, such as 1-alkyl-2,3-trimethyleneimidazolium or 1-alkyl-2,3-tetramethyleneimidazolium); pyridinium-based cations, such as 1-C₁₋₁₀ alkyl-pyridinium; pyrrolidinium-based cations, such as 1-R₁-1-R₂-pyrrolidinium, where each of R₁ and R₂ is independently C₁₋₁₀ alkyl; ammonium-based cations, such as NR₁R₂R₃R₄, where each of R₁, R₂, R₃, and R₄ is independently C₁₋₁₀alkyl; and phosphonium-based cations, such as PR₁R₂R₃R₄, where each of R₁, R₂, R₃, and R₄ is independently C₁₋₁₀ alkyl. Exemplary anions (e.g., such as X for any ionic liquid described herein) include a halogen (e.g., fluoride, bromide, chloride, or iodide); a phosphate anion (e.g., hexafluorophosphate [PF₆], dihydrogen phosphate [dhp], or tris(pentafluoroethyl) trifluorophosphate [FAP]); a borate anion (e.g., tetracyanoborate [TCB], tetrafluoroborate [BF₄], or bis(oxalato)borate [BOB]); a sulfonylimide anion N(SO₂CnF₂ₙ₊₁)(SO₂CₘF₂ₘ₊₁), where each of n and m is, independently, an integer between 1 to 10, and optionally n=m, such as bis(trifluoromethanesulfonyl)imide (N(SO₂CF₃)₂ or [TFSI]) or bis(perfluoroethanesulfonyl) imide (N(SO₂C₂F₅)₂; [BETI] or [PFSI]); a sulfonate anion (e.g., triflate [SO₃CF₃], mesylate [SO₃CH₃], or tosylate [SO₃C₆H₄CH₃]); an alkylsulfate anion (e.g., C₁₋₁₀ alkyl-OSO₃); a cyanimide anion (e.g., [(CN)₂N]); or a carboxylate anion (e.g., formate, acetate, lactate, oxalate, citrate, malate, glycolate, or saccharinate).

Exemplary ionic liquids include choline ionic liquids (e.g., choline dihydrogen phosphate (choline dhp) or choline saccharinate); 1-alkyl-3-methylimidazolium [R-mim] ionic liquids (e.g., such as 1-alkyl-3-methylimidazolium anion [R-mim][X] ionic liquids, including 1,3-dimethylimidazolium iodide, 1-ethyl-3-methylimidazolium bromide, 1-propyl-3-methylimidazolium bromide, 1-propyl-3-methylimidazolium chloride, 1-propyl-3-methylimidazolium bis(trifluoromethanesulfonyl)imide, 1-propyl-3-methylimidazolium bis(perfluoroethanesulfonyl)imide, 1-butyl-3-methylimidazolium bromide, 1-butyl-3-methylimidazolium chloride, 1-butyl-3-methylimidazolium bis(trifluoromethanesulfonyl)imide, 1-butyl-3-methylimidazolium bis(perfluoroethanesulfonyl)imide, 1-pentyl-3-methylimidazolium bromide, 1-hexyl-3-methylimidazolium bromide, 1-heptyl-3-methylimidazolium bromide, 1-octyl-3-methylimidazolium bromide, or 1-nonyl-3-methylimidazolium bromide); (3-methylimidazolium-1-yl)alkanol [ROH-mim] ionic liquids (e.g., such as (3-methylimidazolium-1-yl)alkanol anion [ROH-mim][X] ionic liquids, including 3-(3-methylimidazol-3-ium-1-yl)propan-1-ol bromide, 3-(3-methylimidazol-3-ium-1-yl)propan-1-ol chloride, 4-(3-methylimidazol-3-ium-1-yl)butan-1-ol bromide, 5-(3-methylimidazol-3-ium-1-yl)pentan-1-ol bromide, or 6-(3-methylimidazol-3-ium-1-yl)hexan-1-ol bromide); 1-alkyl-2,3-dimethylimidazolium [R-dmim] ionic liquids (e.g., such as 1-alkyl-2,3-dimethylimidazolium anion [R-dmim][X] ionic liquids, including 1,2,3-trimethylimidazolium iodide, 1-ethyl-2,3-dimethylimidazolium bromide, 1-propyl-2,3-dimethylimidazolium bromide, 1-butyl-2,3-dimethylimidazolium bromide, 1-pentyl-2,3-dimethylimidazolium bromide, 1-hexyl-2,3-dimethylimidazolium bromide, 1-heptyl-2,3-dimethylimidazolium bromide, 1-octyl-2,3-dimethylimidazolium bromide, or 1-nonyl-2,3-dimethylimidazolium bromide); 1-alkyl-2,3-trimethyleneimidazolium [R-3C-im] ionic liquids (e.g., such as 1-alkyl-2,3-trimethyleneimidazolium anion [R-3C-im][X] ionic liquids, including 1-methyl-2,3-trimethyleneimidazolium iodide, 1-ethyl-2,3-dimethyleneimidazolium bromide, 1-propyl-2,3-dimethyleneimidazolium bromide, 1-butyl-2,3-dimethyleneimidazolium bromide, 1-pentyl-2,3-dimethyleneimidazolium bromide, or 1-hexyl-2,3-dimethyleneimidazolium bromide); 1-alkyl-2,3-tetramethyleneimidazolium [R-4C-im] ionic liquids (e.g., such as 1-alkyl-2,3-tetramethyleneimidazolium anion [R-4C-im][X] ionic liquids, including 1-methyl-2,3-tetramethyleneimidazolium iodide, 1-ethyl-2,3-tetramethyleneimidazolium bromide, 1-propyl-2,3-tetramethyleneimidazolium bromide, 1-butyl-2,3-tetramethyleneimidazolium bromide, 1-pentyl-2,3-tetramethyleneimidazolium bromide, or 1-hexyl-2,3-tetramethyleneimidazolium bromide); and 1-butyl-3-methylimidazolium [Bmim] ionic liquids (e.g., such as 1-butyl-3-methylimidazolium anion [Bmim][X] ionic liquids, including 1-butyl-3-methylimidazolium hexafluorophosphate (Bmim PF₆) or 1-butyl 3-methylimidazolium lactate (Bmim lactate)).

In particular examples, the following ionic liquids can be used in combination with a nucleic acid (e.g., DNA and/or RNA): 1-alkyl-3-methylimidazolium [R-mim] ionic liquids (e.g., such as [R-mim][X] ionic liquids or any described herein); (3-methylimidazolium-1-yl)alkanol [ROH-mim] ionic liquids (e.g., such as [ROHmim][X] ionic liquids or any described herein); 1-alkyl-2,3-dimethylimidazolium [R-dmim] ionic liquids (e.g., such as [R-dmim][X] ionic liquids or any described herein); [R-3C-im] ionic liquids (e.g., such as [R-3Cim][X] ionic liquids or any described herein); [R-4C-im] ionic liquids (e.g., such as [R-4C-im][X] ionic liquids or any described herein); or [Bmim] ionic liquids (e.g., [Bmim][X] ionic liquids or any described herein). Further ionic liquid are described in Shi et al., Chem. Commun. 48:5325-5327 (2012), Wang et al., Anal. Chem. 79:620-625 (2007), and Fukaya et al., AE1-Fourteenth International Symposium on Molten Salts Joint International Meeting, Oct. 3-Oct. 8, 2004, “Evaluation of a series of imidazolium based ionic liquids as solvents for nucleic acids,” Abstract 2437, each of which is incorporated herein by reference in its entirety.

Exemplary non-Newtonian fluids include shear-thickening fluids, gels, including hydrogels, and carbohydrate-rich or lipid-rich phases, including lipidic cubic phase and other lipid mesophases. In some cases, permeability to gases may be desirable, for example in some applications that use live cells and tissues inside the device. Exemplary lubricating powders or beads include various Teflon® beads or powders (e.g., composed of PTFE (poly(1,1,2,2-tetrafluoroethylene), PFA (perfluoroalkoxy copolymer resin), or FEP (fluorinated ethylene propylene resin)), graphite, molybdenum disulfide, or tungsten disulfide. Any of these lubricants can optionally include one or more surfactants, for example to cause or prevent surface aggregation and/or to influence the stability of substances.

Reagent Storage

In some embodiments, the integrated device comprises stored reagents. In some embodiments, the liquids in the devices (including those described above) can be stored, such as in blister packs, reagent packs, or other types of containers in which they are sealed.

In some embodiments, the device comprises a reagent pack suitable for preparing nucleic acid of the cell or microorganism for nucleic acid quantification reaction. In some embodiments, the device comprises a reagent pack suitable for nucleic acid quantification reaction. In some embodiments, the reagent pack comprises enzymes for performing a nucleic acid quantification reaction. In some embodiments, the reagent pack comprises primers for performing a nucleic acid quantification (e.g., for PCR or isothermal amplification)

Exemplary reagents in the reagent pack can include, but are not limited to, lysis solutions, wash solutions, elution solutions, rehydration solutions, enzyme solutions (e.g., nucleic acid amplification enzymes, polymerase enzymes, restriction enzymes), buffers, liquid, powder, pellets, a gel, microbeads, probes, primers, nucleic acids, DNA, RNA, polypeptides, nucleoside triphosphates (NTPs), antibodies, a sacrificial reagent or any combination thereof. A sacrificial reagent can comprise an aqueous solution, a lubricant, an oil, an aqueous-immiscible liquid, a gel, a gas, a fluorocarbon oil, a surfactant, gas, air, or any combination thereof. For example, the air can be used to generate air bubble for mixing. As another example, air and immiscible liquid can be used to remove leftover solution (dead volume) in the matrix. Reagents can be mixed to change their composition. For example, one type of buffer can be mixed with another buffer or a dry reagent to change its composition to another buffer.

In some embodiments, the device comprises a reagent pack for assaying enzyme presence and/or activity, such as, for example, monobromobimane, 7-Diethylamino-3-(4′-Maleimidylphenyl)-4-Methylcoumarin, N-(7-Dimethylamino-4-Methylcoumarin-3-yl))Maleimide), NiWa blue (1-Benzyl 4-methyl 5-(tert-butoxycarbonylamino)-2-(2,5-dioxo-2,5-dihydropyrrol-1-yl)terephthalate), NiWa Blue II (Dimethyl 5-acetamido-2-(2,5-dioxo-2,5-dihydropyrrol-1-yl)terephthalate), NiWa orange (Dimethyl 2-amino-5-((2,5-dioxo-2,5-dihydro-1H-prrol-′-yl)ethyl)amino)terephthalate), Ellman's reagent/DTNB (5,5′-dithiobis-(2-nitrobenzoic acid)), Umbelliferone-derived cephalosporins, Fluorescein-derived cephalosporins, Resorufin-derived cephalosporins, Rhodamine-derived cephalosporins, Imipenem, and p-nitrophenol releasing substrates

In some embodiments, the device comprises a reagent pack containing one or more drugs at one or more concentration. In some embodiments, the device comprises a reagent pack containing one or more antibiotics at one or more concentration. In some embodiments, the device comprises a reagent pack containing components suitable for accelerating response of a cell or microorganism to a drug (e.g. quorum sensing molecules, etc.). In some embodiments, the device comprises a reagent pack containing culture media to enhance cell growth.

In some embodiments, the device comprises a reagent pack containing gases or gas mixtures, containing H₂S, CO, and NO. Such gases are known, for example, to affect susceptibility of microorganisms to antibiotics. Such gases can be diluted with a gas mixture which could be anaerobic, aerobic, or microoxic. Such gas mixture can contain CO₂. In some embodiments, the device comprises a reagent pack containing lysis reagents to expose intercellular components.

In some embodiments, these containers avoid evaporation of solutions. In some embodiments, reagents can be released from one or more container by piercing certain regions of the containers, or by selectively opening parts of the containers at the appropriate times. In some embodiments, the generated pressure in the device can release the contents of the blisters/containers; in some embodiments these fluids are transported to other parts of the device. In some embodiments, reagents (such as e.g. amplification reagents) are stored as dried reagents, on device, in blister packs, or in containers (or some combination thereof). The reagents can be lyophilized, stored with sugars (e.g. sucrose, trehalose, among others), and/or stored with beads.

### Assays

The device of the present invention can be used to study and perform a number of assays, including but not limited to coagulation or clotting assays, protein aggregation, protein crystallization (including the use of lipidic cubic phase), crystallization and analysis of small molecules, macromolecules, and particles, crystallization and analysis of polymorphs, crystallization of pharmaceuticals, drugs and drug candidates, biomineralization, nanoparticle formation, the environment (via aqueous and air sampling), culturing conditions (e.g., stochastic confinement, lysis of cells, etc.), drug susceptibility, drug interactions, high throughput screening (e.g., one first substance with many, different second substances, or many, different first substances with many, different second substances), multiplex assays (e.g. PCR, Taqman, immunoassays (e.g., ELISA, FISH, etc.)), flow through immunoassays (e.g. that use capture reagents or, capture regions), digital-single molecule ELISA and digital immunoassays, amplification (e.g., PCR, ligase chain reaction (LCR), transcription mediated amplification (TMA), reverse transcriptase initiated PCR, DNA or RNA hybridization techniques, sequencing, and the like), sandwich immunoassays, chemotaxis assays, ramification amplification (RAM), etc. Exemplary techniques for blood assays, crystallization assays, protein aggregation assays, culturing assays are described in U.S. Pat. Nos. 7,129,091, 6,949,575, 5,688,651, 7,329,485, 6,949,575, 5,688,651, 7,329,485, and 7,375,190; U.S. Pub. Nos. 2007/0172954, 2006/0003439, 2003/0022243, and 2005/0087122; and Int. Pub. Nos. WO 2007/089777 and WO 2009/015390, each of which is incorporated herein by reference in its entireties. The device of the present invention can be used for various syntheses, including catalysis, multistep reactions, immobilized multistep synthesis (e.g., small molecule, peptide and nucleic acid syntheses), solid state synthesis, radioisotope synthesis, etc. Finally, the device of the present invention can be used for purification and enrichment of samples.

The devices and methods described herein can be applied for assays for detection of drug susceptibility or resistance in an organism. The detection can be detection of a signal generated by an assay, for example, an assay to detect a nucleic acid or quantification of a nucleic acid associated with a resistance or susceptibility to a drug in an organism.

An assay can comprise conducting a reaction (e.g., amplification) on a nucleic acid from an organism exposed to a drug and comparing the results of the reaction (e.g., reaction outcome, positive or negative signal generation) to a reaction conducted on a nucleic acid from an organism that has not been exposed to the drug. This can reveal a susceptibility or a resistance of the organism to the drug.

The devices and methods described herein can be applied for assays to detect genetic variation, including differences in genotypes, ranging in size from a single nucleotide site to large nucleotide sequences visible at a chromosomal level. Such genotype or polymorphism analysis can be used for applications including but not limited to early diagnosis, prevention and treatment of human diseases; systematics and taxonomy; population, quantitative, and evolutionary genetics; plant and animal breeding; identifying individuals and populations (paternity and forensic analysis), infectious disease diagnostics and monitoring and surveillance, epidemiology. Examples of some applications of inventions described herein are provided herein. These applications are not limited to the use of any of the methods described herein may be used for these applications, including those using different modulators (e.g., restriction enzymes or oligonucleotides), including inhibitors or promoters.

The devices and methods described herein may be used to perform assays that can comprise genetic fingerprinting (e.g., DNA testing, DNA typing or DNA profiling). This methodology can use the individuality of DNA molecules to distinguish between organisms or to show the relationships between them. For example, restriction fragment length polymorphism (RFLP) analysis can be used for genetic fingerprinting. Detecting RFLPs involves fragmenting a sample of DNA by a restriction enzyme, which can recognize and cut DNA wherever a specific short sequence occurs. The resulting DNA fragments can then be separated by length, for example through an agarose gel electrophoresis, and analyzed, for example by transfer to a membrane via the Southern blot procedure followed by hybridization of the membrane to a labeled DNA probe to determine the length of the fragments which are complementary to the probe. An RFLP occurs when the length of a detected fragment varies between individuals and can be used in genetic analysis. Assays disclosed herein can produce a distinct pattern based on the generation of a specific amplicon and the combination of different restriction enzymes during the amplification method, for example as shown in FIG. 3. This methodology does not require post-amplification treatment for the readout and can generate a specific identity or fingerprint for each analyzed target. Using a panel comprising one or more preloaded restriction enzymes can be used to generate a DNA profile for a specific amplicon or amplicons. Exemplary applications of this assay include epidemiological surveillance (for example, microbiological typing systems for Salmonella spp., Escherichia spp., Staphylococcus spp., Campylobacter spp., Listeria spp. and others); bacterial species are grouped showing maximal similarity phenotypic and genotypic characters, however species may often be subdivided (“typed”) on the basis of characters of a single class (e.g., biotyping, serotyping, phage typing, bacteriocin typing) and practical use of this can be made to obtain information about sources and routes of infection (epidemiological surveillance). Other applications include characterization of genetic patterns associated to health or diseases status (e.g., cancer), detection of drug resistance mutations (e.g., HIV and HCV), and identification of antibiotic resistance (e.g., Methicillin-resistant Staphylococcus aureus (MRSA)) can be accomplished using the methodologies described herein.

The devices and methods described herein can be used for various syntheses, including catalysis, multistep reactions, immobilized multistep synthesis (e.g., small molecule, peptide and nucleic acid syntheses), solid state synthesis, radioisotope synthesis, etc. Finally, the device of the present invention can be used for purification and enrichment of samples. In some embodiments, the device can contain chambers that are used as a positive control (e.g., an analyte pre-loaded in a chamber) and/or a negative control (e.g., a buffer pre-loaded in a chamber). The devices and methods of the invention can be used to conduct any useful reaction. Exemplary, non-limiting reactions include photochemical and electrochemical reactions, chemical reactions such as synthetic reactions (e.g., synthesis of radioisotopes), neutralization reactions, decomposition reactions, displacement reactions, reduction-oxidation reactions, precipitation, crystallization (e.g., protein crystallization by free interface diffusion and/or vapor diffusion), combustion reactions, and polymerization reactions, as well as covalent and noncovalent binding, phase change, color change, phase formation, dissolution, light emission, changes of light absorption or emissive properties, temperature change or heat absorption or emission, conformational change, and folding or unfolding of a macromolecule such as a protein. Multistep reactions may be performed by controlling conditions at each subsequent relative movement of the device.

The devices and methods described herein can be used to perform one or more chemical and/or biochemical reactions. Reactions using the devices and methods described herein can be used to perform synthesis of chemical and biological molecules, perform chemical reactions, perform growth of cells and organisms and/or study interaction of different entities. Analysis and detection of viral, archaeal, bacterial, fungal, mammalian, human, and other nucleic acids can be performed. Such reactions can be used to perform qualitative and quantitative analysis (or steps of such analyses) of a variety of analytes. Analytes may include, but are not limited to viruses, including hepatitis viruses such as HCV, HBV, HAV, HIV, HPV, and other analytes of relevance to human health, agriculture, agricultural biotechnology or practical application. Analysis and detection includes comparative analysis and detection, where a target nucleic acid is compared with another nucleic acid. Such reactions can also be used to perform synthesis of chemical and biological molecules, and grow cells and/or organisms. The process to be modulated can comprise one or more reactions, including but not limited to nucleic acid amplification, reverse transcription, digestion, cloning, ligation, hybridization, phosphorylation, dephosphorylation, glycosylation, deglycosylation, ubiquitination, deubiquitination, S-nitrosylation, denistrosylation, methylation, demethylation, N-acetylation, deacetylation, lipidation, proteolysis, sequencing, or signal generation.

The devices and methods described herein may be used to perform various nucleic acid amplification methods, including modulation methods using restriction enzymes that can use nucleic acid amplification methods wherein the amplification conditions allow the activity of the restriction enzyme to be preserved or partially preserved. The nucleic acid amplification method can comprise polymerase chain reaction (PCR), reverse transcription PCR (RT-PCR), quantitative PCR (qPCR), reverse transcription qPCR (RTqPCR), nested PCR, multiplex PCR, asymmetric PCR, touchdown PCR, random primer PCR, hemi-nested PCR, polymerase cycling assembly (PCA), colony PCR, ligase chain reaction (LCR), digital PCR, methylation specific-PCR (MSP), co-amplification at lower denaturation temperature-PCR (COLD-PCR), allele-specific PCR, intersequence-specific PCR (ISS-PCR), whole genome amplification (WGA), inverse PCR, thermal asymmetric interlaced PCR (TAIL-PCR), or other methods including isothermal amplification methods. Isothermal amplification is a form of nucleic acid amplification which does not rely on the thermal denaturation of the target nucleic acid during the amplification reaction and hence may not require multiple rapid changes in temperature. Isothermal nucleic acid amplification methods can therefore be carried out inside or outside of a laboratory environment. A number of isothermal nucleic acid amplification methods have been developed, including but not limited to Strand Displacement Amplification (SDA), NEAR, Transcription Mediated Amplification (TMA), Nucleic Acid Sequence Based Amplification (NASBA), Recombinase Polymerase Amplification (RPA), Rolling Circle Amplification (RCA), Ramification Amplification (RAM), Helicase-Dependent Isothermal DNA Amplification (HDA), Circular Helicase-Dependent Amplification (cHDA), Loop-Mediated Isothermal Amplification (LAMP), Single Primer Isothermal Amplification (SPIA), Signal Mediated Amplification of RNA Technology (SMART), Self-Sustained Sequence Replication (3SR), Genome Exponential Amplification Reaction (GEAR) and Isothermal Multiple Displacement Amplification (IMDA). Further examples of such amplification chemistries are described in, for example, (“Isothermal nucleic acid amplification technologies for point-of-care diagnostics: a critical review, Pascal Craw and Wamadeva Balachandrana Lab Chip, 2012, 12, 2469-2486, DOI: 10.1039/C2LC40100B,”) incorporated here in its entirety by reference. Isothermal amplification methods that operate at temperatures lower than PCR operating temperatures can be used, e.g., to improve compatibility of restriction enzymes with the amplification process if the restriction enzyme is not sufficiently stable under typical PCR operating temperatures. Furthermore, detection methods based on both signal amplification and target amplification, such as branched-DNA-based detection methodologies, can be used in this approach. For example, for branched-DNA-based detection methodologies, using an enzyme that can cleave the target in a position located between two positions used for binding of the capture extender and the label extender (e.g., as described in Tsongalis, Branched DNA Technology in Molecular Diagnostics, Am J Clin Pathol 2006; 126: 448-453), can reduce the signal obtained in the assay when a restriction enzyme recognizes and cleaves the target.

Devices and methods described herein may be beneficial when analyzing samples with low concentrations of analytes, for example, dilute samples; rare nucleic acids, proteins, markers, and biomarkers of genetic or infectious disease; environmental pollutants; rare cells, such as circulating cancer cells, stem cells, or fetal cells in maternal blood for prenatal diagnostics; microbial cells in blood, sputum, bone marrow aspirates and other bodily fluids such as urine and cerebral spinal fluid for rapid early diagnostics of infections; viral loads (e.g., for HIV and/or HCV) in samples (e.g., in samples from subjects having or suspected of having chlamydia, gonorrhea, and/or HIV); enzymatic assays; cellular assays, such as to determine cell viability, cell adhesion, cell binding etc.; biological or chemical screens for catalytic activity, selectivity, or storage ability or sequestration (such as absorption of gas or trapping of toxic compounds, etc.); or analytical testing various properties such as electrical, magnetic, optical, etc. See e.g., U.S. Pub. Nos. 2005/0003399 and Int. Pub. No. WO 2009/048673, incorporated herein by reference. In particular, detecting low concentrations of an analyte (e.g., a single molecule or a single bacterium) remains a challenge in food, medical, and security industries. The device of the invention could be useful for concentrating such samples and performing analysis. In one example, the devices of the invention can be useful for creating a high local concentration of an analyte (e.g., by compartmentalization within a chamber and/or a droplet or by concentration by using a capture region) that would only be present in dilute concentrations for a bulk solution. In another example, devices of the invention can create high local concentrations of an analyte that can further be amplified, such as by PCR with a DNA sample or by quorum sensing with a bacterial sample. Accordingly, the devices of the invention can be used in combination with any useful PCR technique.

The devices, methods, and systems of the invention can be used to quantify volumes of a sample, a reagent, or any useful substance (e.g., any described herein). In particular, quantification of volumes can be used in combination with any of the other devices and methods described herein, such as for sample preservation, sample treatment, sample preparation, and/or sample analysis. In particular, such volume quantification techniques can be useful for screening of special populations (such as newborns, infants, or small animals, e.g., for screening inherited metabolic disorders or lysosomal storage disorders, such as Fabry, Gaucher, Krabbe, Niemann-Pick A/B, and Pompe disease; for screening viral infections, such as HIV or CMV; or for screening other disorders using useful diagnostic markers, such as screening for succinylacetone, acylcarnitines, and amino acids to detect tyrosinemia type I (TYR 1) in newborns or infants), for use with a dried blood spot (DBS) sample (e.g., in combination with one or more sample preservation and/or storage devices and methods, as described herein), for screening metabolites (e.g., for pharmacokinetic, pharmacodynamic, toxicokinetic, or other drug monitoring assessments), for use in clinical trials (e.g., for pharmacokinetic or pharmacodynamic assessment of investigational drugs in clinical trials), and for determining adherence with particular drugs (e.g., for pharmacokinetic, pharmacodynamic, toxicokinetic, or other drug monitoring assessments). In particular cases, the test sample is a dried blood spot sample. In one non-limiting example, the device including one or more of a membrane, a bridge, a matrix, a capture region, and/or a desiccant (e.g., a device for sample preservation including one or more of a membrane, a bridge, and/or a desiccant) is used, either with or without a collector, and a blood sample is introduced into the device. Next, the blood sample is dried (either partially or completely, e.g., as described herein). In some cases, the blood sample is dried onto a cellulose membrane that is optionally in fluidic communication with a desiccant. Then, the dried blood sample is processed and/or analyzed using one or more useful substances or reagents. Exemplary substances or reagents include a buffer (e.g., a wash buffer or an elution buffer, e.g., PBS containing 0.05% Tween 80 and 0.005% sodium azide, or any described herein), such as those used for screening in DBS technology, including amplification (e.g., PCR); detection of a virus, bacteria, protozoa, and/or helminth (e.g., HIV, hepatitis C virus, hepatitis B virus, hepatitis A virus, herpes simplex virus, rubella, measles, MMR (measles, mumps, and rubella), diphtheria, dengue, tetanus antitoxin, cytomegalovirus, human T-cell leukemia/lymphoma virus I or II, Mycobacterium leprae, Helicobacter pylori, Brucella sp, Treponema pallidum, Toxoplasma gondii, Plasmodium falciparum, Trypanosoma cruzi, Giardia lamblia, Leishmania spp, Echinococcus granulosus, Schistosoma haematobium, or Brugia malayi); detection of one or more metabolites (e.g., drug metabolites); detection of one or more analytes (e.g., any described herein, and including androstenedione, amino acids (e.g., arginine (Krebs cycle), histidine/urocanic acid, homocysteine, phenylalanine/tyrosine, and/or tryptophan), apolipoprotein (e.g., A-I or B), cortisol, CD4+ lymphocytes, cholesterol (e.g., including total cholesterol or high-density lipoprotein cholesterol (HDL)), C-reactive protein (CRP), dehydroepiandrosterone (DHEA, including its sulfate ester, DHEA-S), Epstein-Barr virus (EBV) antibodies, estradiol, folate, follicle-stimulating hormone (FSH), glucose, hemoglobin (e.g., including glycosylated Hemoglobin or HbAlc), hepatitis antigen/antibodies (e.g., hepatitis A, B, or C), HIV antibodies, homocysteine, IFNg, IGF-I, IGFBP-2, IGFB-3, IL-1b, IL-6, insulin, leptin, luteinizing hormone (LH), lipoprotein (e.g., (a), B/A-1 or β), prostate-specific antigen (PSA), progesterone, prolactin, retinol, sex hormone binding globulin (SHBG), somatomedin-C, testosterone, transferrin receptor, thyrotropin (TSH), thyroxine (T4), thyroglobulin, triglycerides, triiodothyronine (T3), or TNF (e.g., TNFa)); detection of one or more diagnostic markers for special populations, such as a newborn, a neonate, or an infant (e.g., detection of IgG antibodies for diagnosing infections; detection of succinylacetone, acylcarnitines, and amino acids for diagnosing tyrosinemia type I (TYR 1); detection of medium chain acyl CoA dehydrogenase for diagnosing MCAD deficiency; detection of human chorionic gonadotropin (hCG) for diagnosing Down syndrome; detection of glycated hemoglobin for diagnosing insulin-dependent diabetes; detection of trypsin for diagnosing cystic fibrosis; detection of HIV-specific antibodies and/or of HIV virus in combination with PCR; detection of thyroxine (T4) and thyrotropin (TSH) for diagnosing congenital hypothyroidism; detection of one or more enzymes (e.g., acid α-glucocerebrosidase (ABG), acid α-galactosidase A (GLA), lysosomal acid α-glucosidase (GAA), galactocerebroside α-galactosidase (GALC), or acid sphingomyelinase (ASM)) involved in lysosomal metabolism for diagnosing lysosomal storage disorders (e.g., Pompe, mucopolysaccharidosis (e.g., type I), Fabry, Gaucher, or Niemann-Pick type A/B diseases); for DNA analysis in combination with PCR analysis (e.g., for detecting or diagnosing acetylator polymorphism, alcohol dehydrogenase, alpha 1-antitrypsin, cystic fibrosis, Duchenne/Becker muscular dystrophy, glucose-6-phosphate dehydrogenase, hemoglobinopathy A, hemoglobinopathy S, hemoglobinopathy C, hemoglobinopathy E, D-Punjab, beta-thalassemia, hepatitis B virus, HCMV, HIV-1, HTLV-1, Leber hereditary optic neuropathy, MCAD, PKU, Plasmodium vivax, sexual differentiation, or 21-deoxycortisol); for detecting certain antigens (e.g., hepatitis B virus or HIV-1); for detecting certain antibodies (e.g., adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella Mycobacterium leprae, Mycoplasma pneumoniae, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli vesicular stomatis virus, Wuchereria bancrofti, or yellow fever virus); or screening of one or more drug metabolites or drug analytes (e.g., for pharmacokinetic, pharmacodynamic, toxicokinetic, or other drug monitoring assessments in clinical trials, in clinical monitoring, or in determining adherence with particular drugs, where exemplary drugs include anti-cancer drugs such as everolimus or tacrolimus; acetaminophen; investigational new drugs; or others). Further analytes, DBS assays, and methods are described in McDade et al., Demography 44:899-925 (2007); Cassol et al., J. Clin. Microbiol. 29:667-671 (1991); Bellisaro et al., Clin. Chem. 46:1422-1424 (2000); Williams et al., J. Gerontol. B Psychol. Sci. Soc. Sci. 64B(suppl_1): i131-i136 (2009); Parker et al., J. Clin. Pathol. 52:633-639 (1999); Li et al., Biomed. Chromatograph. 24:49-65 (2010); and De Jesus et al., Clin. Chem. 55:158-164 (2009), each of which is incorporated herein in its entirety.

The devices and methods described herein may be used to perform assays that can be used in the identification of single point mutations, for example for viral genotyping. Genotyped viruses can include but are not limited to hepatitis C virus, hepatitis B virus, human immunodeficiency virus, human cytomegalovirus, norovirus and enterovirus. Assays can be used for viral typing and subtyping. Typed or subtyped viruses can include but are not limited to human papilloma virus, avian influenza virus, human influenza virus, swine influenza virus, herpes simplex virus, foot and mouth disease virus, dengue virus and rotavirus. Assays can be used for bacterial typing. Typed bacteria can include but are not limited to Francisella spp., Escherichia spp., Salmonella spp., Mycobacterium spp., Bacillus spp., Staphylococcus spp., Streptococcus spp., Acinetobacter spp., Helicobacter spp., Bordetella spp., Bordetella spp. and Vibrio spp. Assays can be used to assess for the presence or absence of drug resistance mutations, in subjects including but not limited to human immunodeficiency virus, hepatitis C virus, and cancer drug resistance.

The devices and methods described herein may be used to perform assays with results that can comprise a readout or detection mechanism chosen from a range of readouts used to detect progress or results of reactions. Examples include but are not limited to electrochemical readouts, optical readouts, including for example fluorescence readouts, colorimetric readouts, chemiluminescence, electrical signals, quenching, probe binding, probe hybridization, metal labeling, contrast agent labeling, absorbance, mass spectrometry, sequencing, lateral flow strips, and the generation of a heterogeneous substance (e.g., precipitation, gas bubble).

Devices and methods described herein may include tubes, capillary tubes, droplets, microfluidic devices (e.g., SlipChip devices), wells, well plates, microplates, microfluidic wells, microfluidic droplets, emulsions, solid supports (e.g., beads or microarrays), microchips, or gels (e.g., 2D gels, 3D gels) and reactions inside gels including “polonies” as in polony PCR on surfaces and in gels.

The devices and methods described herein may be used to perform an assay that can be conducted in less than or equal to about 600 minutes, 540 minutes, 480 minutes, 420 minutes, 360 minutes, 300 minutes, 240 minutes, 180 minutes, 120 minutes, 110 minutes, 100 minutes, 90 minutes, 80 minutes, 70 minutes, 60 minutes, 50 minutes, 40 minutes, 30 minutes, 20 minutes, 15 minutes, 10 minutes, 9 minutes, 8 minutes, 7 minutes, 6 minutes, 5 minutes, 4 minutes, 3 minutes, 2 minutes, or 1 minute. An assay can have an accuracy of at least about 50%, 60%, 70%, 80%, 90%, 95%, 96%, 97%, 98%, 99%, 99.9%, or 99.99%. The rates of false positives can be below 10%, below 1%, below 0.1%, below 0.01%, below 0.001%, or below 0.0001%. The rates of false negatives can be below 10%, below 1%, below 0.1%, below 0.01%, below 0.001%, or below 0.0001%.

The devices and methods described herein may be used to perform assays that can be used to identify drug resistance mutations (DRMs). Drug resistance can be achieved by multiple mechanisms, including but not limited to horizontal acquisition of resistance genes (carried by plasmids or transposons), by recombination of foreign DNA into the chromosome, or by mutations in different chromosomal loci. The identification of a characteristic restriction enzyme pattern or the activity of specific restriction enzymes associated with mutations that confer drug resistance can, in some cases, be accomplished using the methodologies described herein. Drug resistance can be determined using the methodologies described herein in subjects including but not limited to viruses, bacteria, fungi, plants, prokaryotes, and eukaryotes. These mutations can also be determined in, e.g., cancer cells and cell-free DNA. For example, this can be applied to identify drug resistance mutations in HCV (see, e.g., “Clinically Relevant HCV Drug Resistance Mutations Figures and Tables, from HCV Phenotype Working Group, HCV Drug Development Adivosry Group, Ann Forum Collab HIV Res. Volume 14 (2): 2012; 1-10” or “Forum for Collaborative HIV Research, University of California Berkeley School of Public Health”), HIV (see, e.g., “Victoria A. Johnson, MD, Vincent Calvez, MD, PhD, Huldrych F. Günthard, MD, Roger Paredes, MD, PhD, Deenan Pillay, MD, PhD, Robert W. Shafer, MD, Annemarie M. Wensing, MD, PhD, and Douglas D. Richman, MD, Update of the Drug Resistance Mutations in HIV-1: March 2013, Topics in Antiviral Medicine, 2013; 21:6-14”), and influenza A virus (see, e.g., “Goran Orozovic, Kanita Orozovic, Johan Lennerstrand, Bjorn Olsen, Detection of Resistance Mutations to Antivirals Oseltamivir and Zanamivir in Avian Influenza A Viruses Isolated from Wild Birds. PLoS ONE 6(1): e16028. doi:10.1371/journal.pone.0016028”).

The devices and methods described herein may be used to perform assays for genetic testing, including fetal genetic testing. For example, assays can be used for non-invasive prenatal Trisomy 21 (Down syndrome) diagnostics. Assays can be used with a screening test which indicates the likelihood of trisomy. Assays can be used with or as a screening test for a subsequent diagnostic test which is a more accurate test provided only to people with a high score in the screening test. A screening test can comprise an ultrasound test. A screening test can comprise a maternal serum screening blood test measuring the level of human chorionic gonadotropin (β-hCG), pregnancy associated plasma protein-A (PAPP-A), alpha fetoprotein (AFP), or other protein biomarkers. A screening test can provide a probability instead of a finite answer. If the screening test gives a high score, an invasive diagnostic test, such as chorionic villus sampling (CVS) or amniocentesis, can be used. Cell-free fetal DNA (cff DNA) or RNA (cff RNA) exist in maternal plasma that can be isolated and subjected to molecular analysis (see, e.g., “Y M Dennis Lo, Noemi Corbetta, Paul F Chamberlain, Vik Rai, Ian L Sargent, Christopher W G Redman, James S Wainscoat, Presence of fetal DNA in maternal plasma and serum, Lancet 1997, 350, 485-487”). The cff DNA and cff RNA can be used in assays for non-invasive biomarker discovery and detection. For cff DNA, a biological and therefore technical constraint is that it only takes up 3-6% of the total amount of cell-free DNA. The proportion increases at a later stage of gestation, but is still a minor fraction of the total amount of cell-free DNA in plasma. Assays can directly target fetal-specific DNA or RNA. The placenta is an organ that can represent genetic information from the fetus. Placental-specific RNA, when expressed and released in a tissue-specific manner, qualifies as a fetal-specific RNA because it exists only in pregnant individuals and does not exist before or after pregnancy. Assays can comprise detection of trisomy 21 using placental specific RNA by direct dosage-related difference in the expression of chromosome 21 encoded genes (see, e.g., “Chi-Ming Li, Meirong Guol, Martha Salas, Nicole Schupf, Wayne Silverman, Warren B Zigman, Sameera Husain, Dorothy Warburton, Harshwardhan Thaker, and Benjamin Tycko, “Cell type-specific over-expression of chromosome 21 genes in fibroblasts and fetal hearts with trisomy 21” BMC Medical Genetics 2006, 7: 24”). Assays can comprise detection of trisomy 21 using placental specific RNA by relative RNA allelic ratio assessment using SNP analysis (see, e.g., “Y M Dennis Lo., Nancy B Y Tsui, Rossa W K Chiu, Tze K Lau, Tse N Leung, Macy M S Heung, Egeliki Gerovassili, Yongjie Jin, Kypros H Nicolaides, Charles R Cantor, and Chunming Ding, Plasma placental RNA allelic ratio permits noninvasive prenatal chromosomal aneuploidy detection, Nat. Med. 2007, 13, 218”). Relative RNA allelic ratio assessment using SNP analysis can quantify the relative abundance of each allele in expressed placental specific RNA when there is a heterozygous loci on chromosome 21 genes, with the assumption that the ratio of the two alleles for mothers carrying a trisomy 21 baby should be 2:1, and for a normal baby it should be 1:1. Since only a relative ratio is required, the number of wells or other partitions can be small if digital PCR is used in such an assay.

The devices and methods described herein may be used to perform assays for epigenetic testing for diseases and other conditions, including but not limited to Angelman syndrome, Prader-Willi syndrome, Beckwith-Wiedemann syndrome, aberrant DNA methylation associated with cancer (hypermethylation, e.g. at CpG islands in the promoter region or hypomethylation, e.g. global hypomethylation), epigenetic changes (e.g., CpG island methylation) associated with reduced expression of DNA repair genes (e.g., BRCA1, WRN, FANCF, RAD51C, MGMT, MLH1, MSH2, ERCC1, Xpf, NEIL1, FANCB, MSH4, ATM), and variant histones.

The devices and methods described herein may be used to perform a rapid assay not requiring CVS or amniocentesis that can be used as a screening test or a diagnostic test with high sensitivity and specificity based on, in one example, RNA SNP quantification on chromosome 21. In some cases, this platform can have the capacity for collecting sample and purifying RNA out of the plasma sample, multiplexed SNP ratio quantification, and a relatively simple readout module, including but not limited to one that is cell-phone enabled. The results can be interpretable by, for example, the user or the physician, and in some cases can be offered together with consultation service at the clinic, to help parents make decisions and prepare.

The devices and methods described herein may be used to perform an assay with coverage that can be at least about 50%, 60%, 70%, 80%, 90%, 95%, 96%, 97%, 98%, 99%, 99.9%, 99.99%, or 100%. An assay can be used in combination with another screening test and can decrease the number of false-positives in the screening test. An assay can provide access to such tests to people who do not currently have access. The development of such a platform can fill the gaps between the large demand for trisomy 21 detection and limited access to diagnostics. In one example of this platform, a sample (such as, for example, human blood) can be collected and loaded onto the integrated device. In some cases, the sample can flow to merge with different reagents to complete the RNA extraction step driven by, for example, pressure. In some cases, the RNA can then be mixed with isothermal amplification reagents containing, for example, different inhibitors specific to different alleles. In some cases, after rapid sequence specific amplification, the digital counts for different reactions can be recorded (such as, for example, by a cell phone, which can have a built-in analysis application). In some cases, the assay can comprise a SNP assay for multiple loci, increasing the population coverage of the entire assay. In some cases, proper control and potentially other trisomy detection assays can be included as well. In some cases, for heterozygous loci, the ratio between the two alleles should be 1:1 for euploidy, while that for aneuploidy should be about 2:1. In some cases, for homozygous loci, only one allele will be detected.

In one embodiment, the devices can be used with sample preparation technology to isolate bacterial DNA and RNA rapidly and in high yield from target microorganisms or cells, such as for example Klebsiella pneumoniae, Pseudomonas aeruginosa, and extra-intestinal pathogenic E. coli and can include clinical samples and can include a variety of bodily fluids, such as for example urine. Sample-prep SlipChip for bacterial DNA and RNA extraction and purification from urine samples containing Chlamydia trachomatis has previously been validated. Viral RNA extraction and purification from plasma samples containing HCV or HIV viral particles has also been validated. In one embodiment, the devices can be used to perform sample preparation in less than 5 min and with samples handling a range of volumes, such as for example up to 0.5 mL. In some embodiments, the device can be used for rapid detection of samples containing low bacterial loads.

In some embodiments, the devices can be used to extract and purify nucleic acids, such as bacterial DNA and RNA from samples (e.g. a urine sample) spiked with a bacterial target (such as for example K. pneumoniae, P. aeruginosa, or E. coli) in some cases in an extremely short period of time (such as for example less than 5 minutes) and in some cases with a yield above 80% compared to standard bench methods, and at a quality suitable for both digital and real-time quantification.

Bacterial RNA is known to be unstable and expression levels can change rapidly. In cases where the quantity and quality of purified RNA can be compromised, the devices described in this disclosure can be used to further shorten the sample preparation protocol, such as for example to less than 3 min. This time frame has previously been demonstrated for isolation of viral RNA from blood plasma. In some embodiments, the lysis step can be modified by adding additional detergents or inhibitors to minimize the activity of RNase.

In some embodiments, the device can be used for quantification of gene expression level for at least 20, at least 50 or at least 100 clinical isolates with incubation in the presence and absence of selected drugs In some embodiments, the device performance, including bacterial load and AST, is comparable to CLSI reference methods. In some embodiments, the error of performance is less than 5% minor errors, less than 2.5% minor errors, less than 1% minor errors. In some embodiments, the performance has, less than 1% major errors, less than 0.5% major errors, less than 0.1% major errors. In some embodiments, the device has no very major errors.

### Kits

A kit can include an integrated device, and a supply of a reagent selected to participate in nucleic acid amplification. In some embodiments, the reagent can be disposed in a container adapted to engage with a conduit of the first component, the conduit of the second component, or both. Such a container can be a pipette, a syringe, and the like. In some embodiments, the kit includes a heater.

Some embodiments of the device could be used to detect different biological targets such as, for example, proteins, bacteria, viruses, infectious agents etc., using nucleic acid labels. In some embodiments the target is tagged with an oligonucleotide which can be used for detection. The oligonucleotide tag can be further amplified using any one of a number of different nucleic acid amplification strategies, such as for example, PCR, LAMP, RPA, NASBA, RCA, etc. The oligonucleotide tag could also be visualized using fluorescent probes for example as shown by Chen (Huang, Suxian, and Yong Chen.“Polymeric Sequence Probe for Single DNA Detection.” Analytical chemistry 83.19 (2011): 7250-7254.)

### EXAMPLES

These examples are provided for illustrative purposes only and not to limit the scope of the claims provided herein.

### Example 1: Fabrication of an Integrated Device

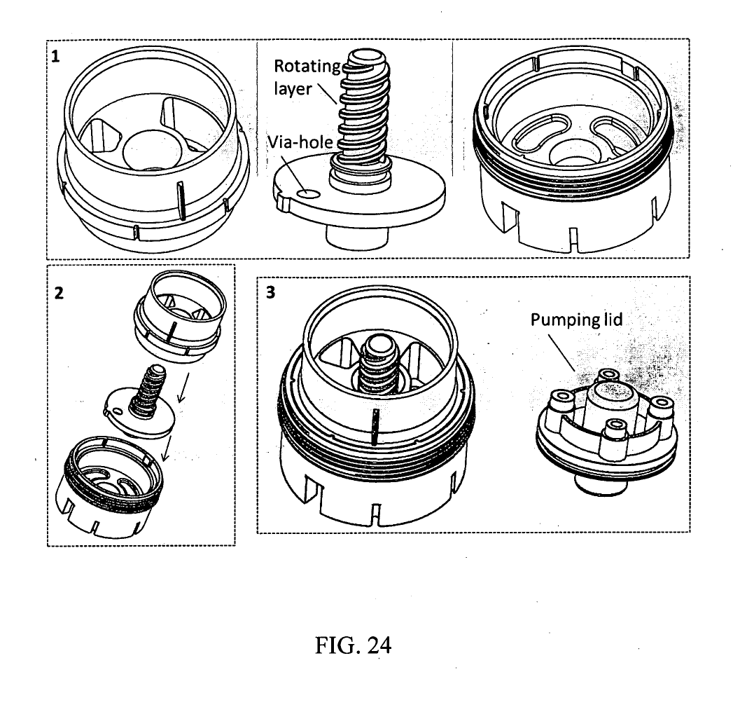

An integrated device was made by using a Stratasys Objet260Connex multi-material 3D printer and using Veroclear material (Stratasys) for printing. The device consists of a DNA/RNA extraction module (see FIG. 24), a pumping lid (right part of panel 3 in FIG. 1) and a constant torque spring motor. The DNA/RNA extraction module contains 3 layers (see panel 1 in FIG. 1), of which the middle layer is the rotating layer that contains a threaded shaft and a via-hole through which solutions can be pumped. The middle layer is rotated by a constant torque spring, while the two other layers are fixed in space. The device also contains a pumping lid, which contains the negative thread of the thread present on the threaded shaft attached to the middle layer of the DNA/RNA extraction module. The device validates the principle of generating pressure in a sealed environment using a constant torque spring.

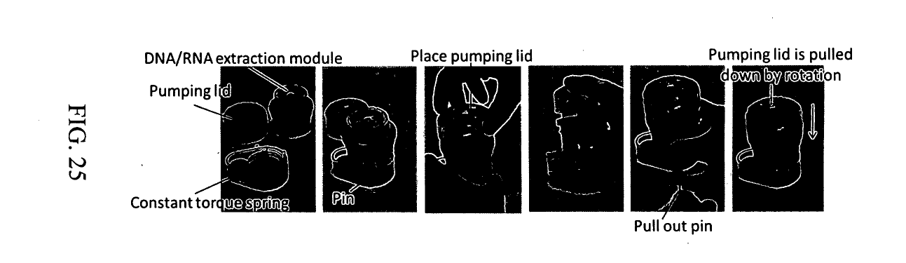

The device was assembled and operated as illustrated in FIG. 24. Photographs of the resulting device are shown in FIG. 25. The DNA/RNA extraction module was placed on top of a constant torque spring. The constant torque spring was pre-wound and held into place by a pin. The pumping lid was placed on top of the DNA/RNA extraction module to create a sealed environment inside of the device. Subsequently, the pin was pulled out of the device to initiate rotation of the constant torque spring. The constant torque spring then rotates the middle layer of the DNA/RNA extraction module, which pulls down the pumping lid by rotation and increases the pressure inside of the DNA/RNA extraction module to pump solutions. These photographs in FIG. 25 demonstrate the assembly of the device, and how it successfully pulls down the pumping lid by rotation of a constant torque spring in order to generate pressure in a sealed environment inside the device. The device validates the principle of generating pressure in a sealed environment using a constant torque spring.

### Example 2: Fluid Management in the Integrated Device

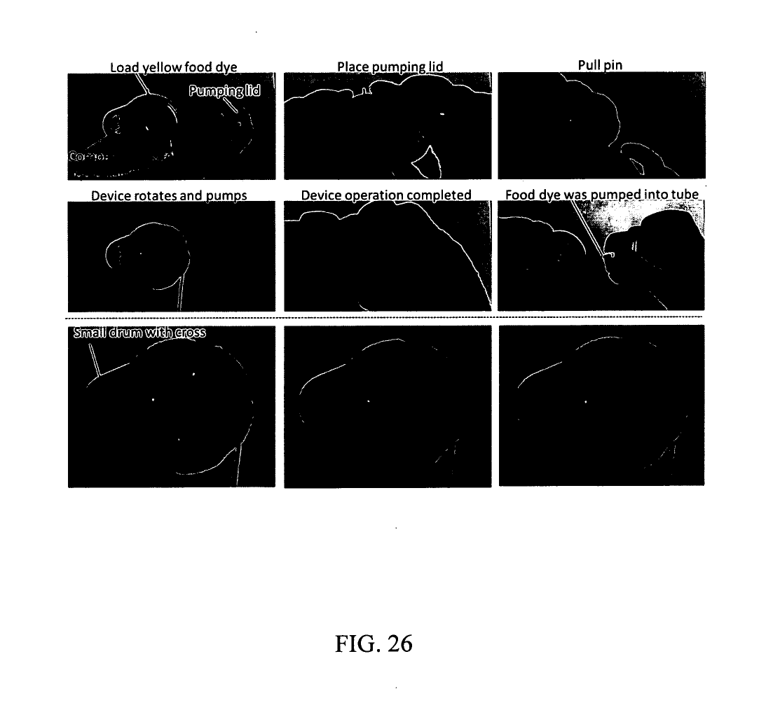

To illustrate the successful pumping of liquid solutions by the device, the device fabricated in Example 1 was used for pumping liquid, yellow food dye. The food dye was loaded into 3 wells of the device by transferring 1.5 mL, 0.8 mL and 0.2 mL into 3 different wells/compartments on a first layer of the integrated device (FIG. 26). After food dye was loaded, the pumping lid was placed on top of the DNA/RNA extraction module to create a sealed environment. The pin was pulled out of the constant torque spring motor, which initiated the rotation of the constant torque spring. The middle layer of the DNA/RNA extraction module started rotating, which caused the pumping lid to be pulled down. This caused the air in the device to be compressed, thereby creating an increase in pressure, which generated an increase in resistance to rotation. As such, the rotation of the middle layer is slowed down. After 10 seconds, the middle layer completed the rotation from the starting position to the position in which the first solution is pumped through the via-hole. Liquid food dye in the first liquid compartment was pumped out of the device through the via-hole in the rotating layer. After all the solution (1.5 mL) is pumped out of the first compartment, which took 3 seconds, air is free to escape through the via-hole, which decreases pressure and resistance to rotation, thereby speeding up rotation of the middle layer again. As the via-hole in the middle layer reaches the second compartment containing liquid solution (10 seconds after pumping out the first solution), pressure is built up again, which slows down rotation and allows liquid food dye solution (0.8 mL) to be pumped out of the second compartment (which takes 3 seconds). This process is repeated once more to pump liquid yellow food dye (0.2 mL) out of the third and last compartment of the device.

The yellow food dye out of the last compartment was captured into a transparent tube (last panel of FIG. 26), demonstrating the successful operation of the integrated device to control movement of fluid in a multi-step process. The bottom row of photographs in FIG. 26 represent video snapshots separated by one second in time to show a close-up of the rotation of the device. A cross was added to the small drum of the constant torque spring motor to indicate rotation. The complete rotation/operation of the device took ˜1 min to complete.

### Example 3: Coupling an Extraction Module to an Amplification Module

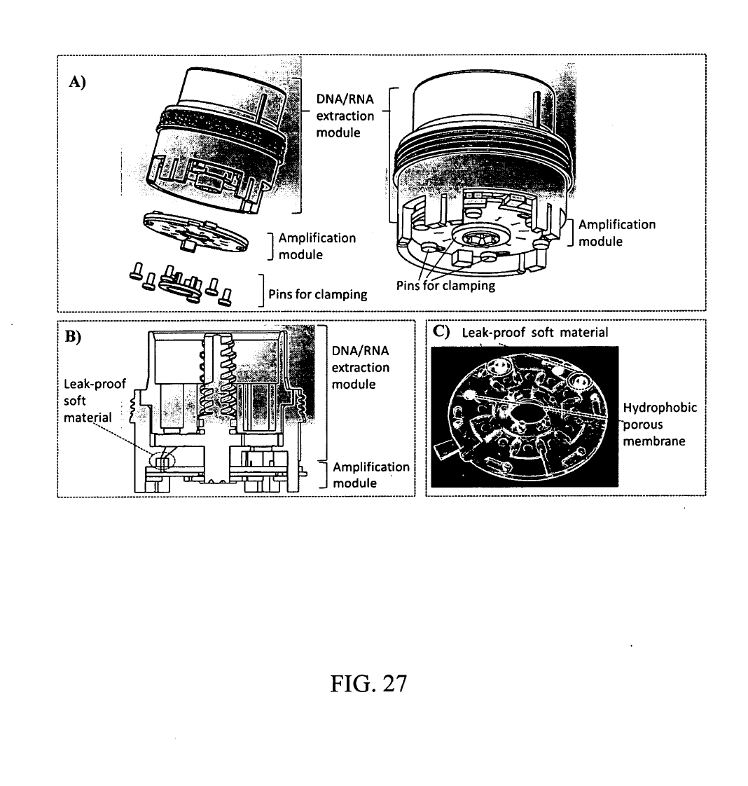

As shown in FIG. 27, the device uses pins to ensure clamping of top and bottom parts of the amplification module and the sample preparation module. In Panel A, an exploded view is shown to demonstrate how the DNA/RNA extraction module and the amplification module are integrated, and clamped together with pins. The assembled DNA/RNA extraction module and amplification modules are also shown in Panel A. In Panel B, a side view through the device is depicted to demonstrate the integration of a DNA/RNA extraction and an amplification module. The liquid transfer from DNA/RNA extraction module to amplification module is achieved by a leak-proof soft material. In Panel C, a photo of an amplification module is depicted, indicating the leak-proof soft material, and a hydrophobic porous membrane for accurate fluid metering. In some embodiments, connections between modules consists of a soft material, which can be, but is not limited to, silicone, polydimethylsiloxane or TangoPlus (from Stratasys). To ensure successful fluid loading and enable accurate fluid metering, air is evacuated through a hydrophobic, porous membrane, such as for example, but not limited to, PTFE membrane (for example, 0.45 μm pore size) while blocking aqueous solution when it contacts the membrane (FIG. 27).

### Example 4: Automated Transfer of Fluid from the Extraction Module to the Amplification Module

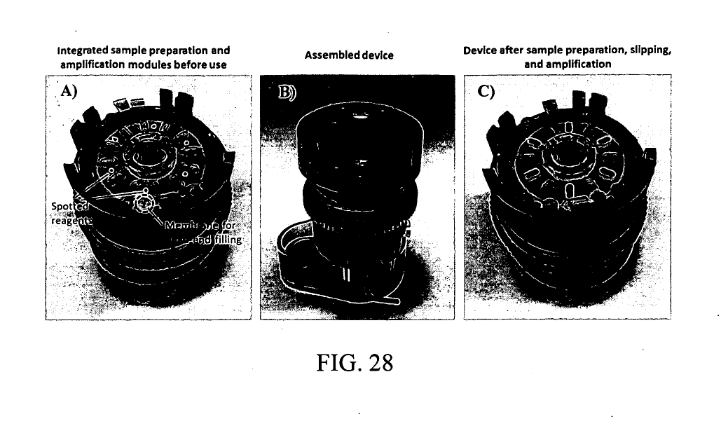

The integrated device automatically transferred liquid solution, represented by a yellow food dye, from the DNA/RNA extraction module, into the attached amplification module, as shown in the photographs of FIG. 28. The DNA/RNA extraction module described in Examples 1 and 2 was used in this experiment. This DNA/RNA extraction module was integrated with an amplification module as shown in FIG. 27 and described in Example 3. Rotation of the middle layer of the DNA/RNA extraction module was performed as described in Example 2 with a constant torque spring. 1.5 mL and 0.8 mL of water were dispensed in the first and second compartment of the DNA/RNA extraction module, and 0.2 mL of green food dye was dispensed in the third compartment of the DNA/RNA extraction module. After these solutions were dispensed, the pumping lid was placed on top of the device and the pin was pulled to start the rotation of the torque spring motor. After the first two liquids were pumped through the via-hole, the green food dye was pumped through the via-hole and directly into the amplification module. One plate of the amplification module slipped relative to each other, enabling straightforward and automated mixing of the purified nucleic acids with pre-stored lyophilized reagents for amplification (which are represented by green food dye in FIG. 28).

Photos illustrating the operation of the device described above are shown in FIG. 28. The device automatically transfers liquid solution resulting from the DNA/RNA extraction module (represented by yellow food dye) to the amplification module where it is automatically mixed with on-device stored reagents (represented by green food dye).

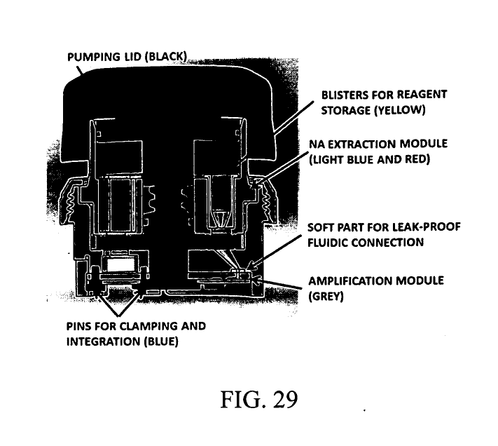

FIG. 29 shows another schematic cross-sectional side view of the integrated device including the pumping lid, indicating the soft parts used for successful leak-proof liquid connection between the DNA/RNA extraction and amplification modules, and pins used for clamping both together.

### Example 5: Extraction of RNA from Chlamydia Trachomatis Culture Using the Automated Integrated Device

The autonomous, integrated and stand-alone sample preparation module was validated by extracting RNA from a purified Chlamydia trachomatis culture sample. A DNA/RNA extraction module as described in Examples 1 and 2 was used, using a constant torque spring for rotation, pressure generation, and fluid flow. For validating this device's performance for nucleic acid extraction, the device was used to purify 16S rRNA from a Chlamydia trachomatis culture.

In a conical centrifuge tube, 1 mL of commercial lysis buffer was mixed with 0.5 mL of Chlamydia trachomatis in phosphate buffered saline solution. This mixture (1.5 mL) was dispensed in the first compartment of the DNA/RNA extraction module. In the second compartment, 0.8 mL of washing buffer (70% ethanol in water) was loaded, and in the third compartment, 0.2 mL of water was loaded. Subsequently, the pumping lid was placed on top of the device, as described in FIG. 2 and FIG. 3. In the middle layer of the DNA/RNA extraction module, a silica-based column for DNA/RNA-purification was placed inside the via-hole (see FIG. 1). The device operated as described in Example 2. When the third solution (water) was pumped through the silica-based column for DNA/RNA-purification, the captured nucleic acids were eluted from the silica-based column for DNA/RNA-purification. The purified RNA was collected from the device in a tube and used for RT-LAMP amplification.

DNA/RNA extraction performed in the integrated device was compared to a standard manual off-device extraction in parallel. The off-device extraction consisted of pipetting the same reagents used on the device in a commercial silica-based column for DNA/RNA purification that was placed in a polypropylene tube, as purchased. First, the 1.5 mL mixture (0.5 mL of Chlamydia trachomatis in phosphate buffered saline mixed with 1 mL of lysis buffer) was pipetted on top of the column, and a 25-mL syringe was manually placed on top of the polypropylene tube to push the solution through the column. This process was repeated twice for subsequently pushing 0.5 mL washing buffer (70% ethanol in water) and 0.2 mL elution buffer (water) through the silica-column.

Chlamydia trachomatis RNA extracted with on-device and off-device methods was amplified by RT-LAMP. The RT-LAMP mix solution contained: 10× in-house LAMP reaction mix (Tris-HCl 200 mM pH 8.8, KCl 100 mM, MgSO4 80 mM, (NH4)2SO4 100 mM, 1% Tween20), Betaine, Bst 2.0 WarmStart® DNA Polymerase, AMV Reverse Transcriptase, Bovine Serum Albumin, Deoxynucleotide Solution Mix, 10× Chlamydia Trachomatis LAMP primer mixture (20 μM BIP/FIP, 10 μM LB/LF, and 2.5 μM B3/F3), 20× EvaGreen dye, nuclease-free water, and on-device or off-device purified Chlamydia trachomatis RNA. 10 μL of positive and negative solution were loaded into 96-wells plate and heated at 63° C. for 50 min and 85° C. for 5 min (heat inactivation) on a LightCycler® 96 Real-Time PCR System.

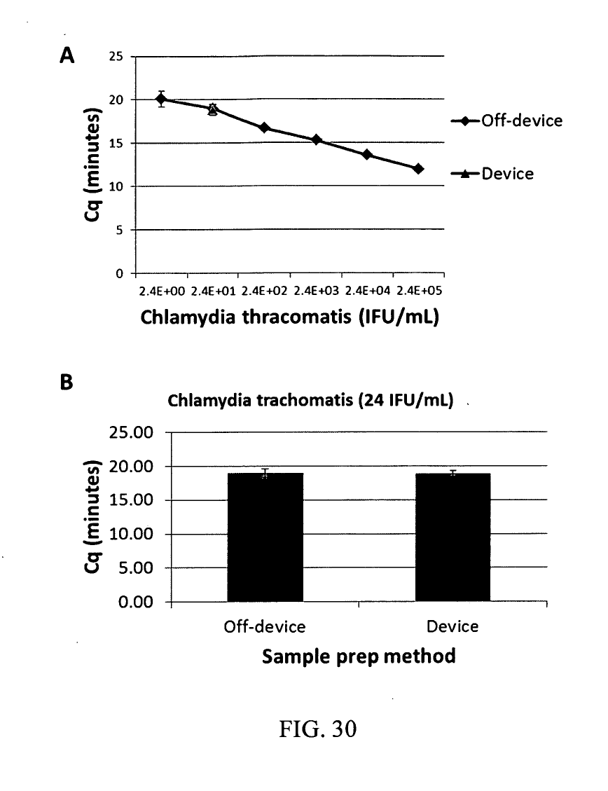

FIG. 30 shows a graph for detection of Chlamydia trachomatis in buffer on an integrated device (triangle shape), compared to off-device detection (square shape) (N=3 for all experiments) in Panel A. Panel B shows a bar chart comparing detection of Chlamydia trachomatis (24 IFU/mL) on the autonomous device and the off-device method (N=3). RT-LAMP was able to detect 24 IFU/mL (IFU=infectious units) of Chlamydia trachomatis (N=3) from the off-device purified RNA. As can be seen in FIG. 30, the autonomous device purifies nucleic acids with a yield sufficient to also detect 24 IFU/mL within 20 min (N=3), showing no significant difference between the off-device protocol and the autonomous device.

### Example 6: Primers for RT-LAMP Detection of C. trachomatis and N. gonorrhoeae 16S rRNA

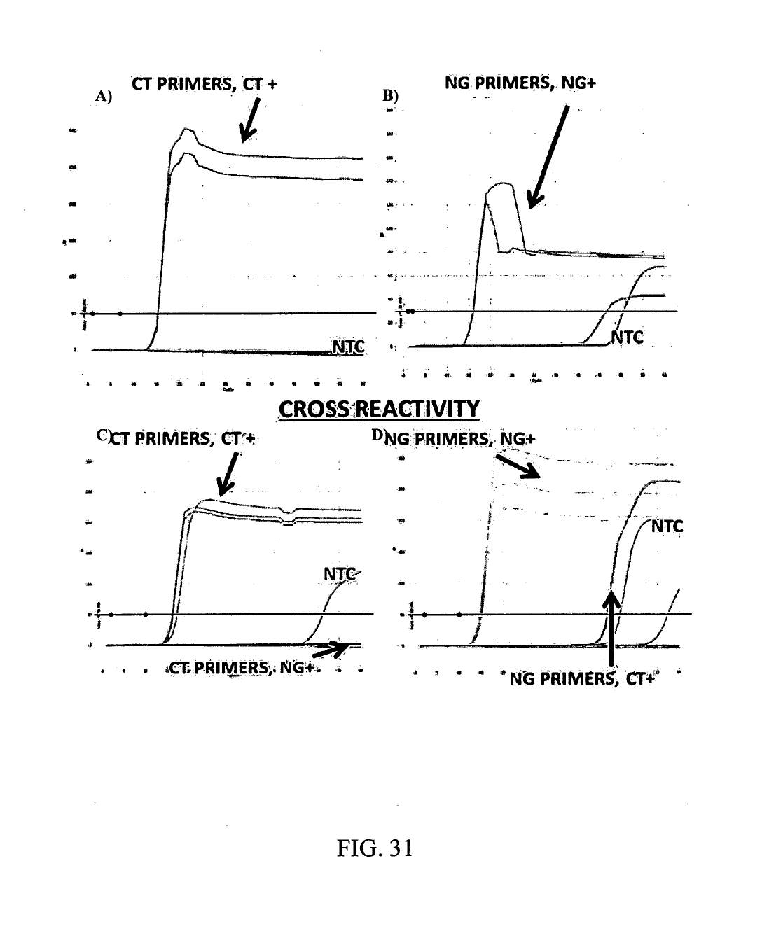

Primers for RT-LAMP detection of Chlamydia trachomatis (CT) or Neisseria gonorrhoeae (NG) 16S rRNA were performed using the primers shown in Table 1 and Table 2. Experimental results showing specificity and no cross reactivity are reported in FIG. 31.

TABLE 1
LAMP primers for Chlamydia trachomatis (CT)

| CT 1st generation | 5′ To 3′ Primer Sequence |
|---|---|
| F3_CT2 | GATTGGCCGCCAACACTG |
| B3_CT2 | CCGGTGCTTCTTTACCTGG |
| FIP_CT2 | GTCAGACTTCCGTCCATTGCGATTTTTGAC |
| | TGAGACACTGCCCAGA |
| BIP_CT2 | GACGCCGCGTGTGTGATGAATTTTTCAGCG |
| | GGTATTAACCGTCTT |
| LF_CT2 | ACTGCAGCCTCCCGTAGGAG |
| LB_CT2 | GGCTCTAGGGTTGTAAAGCACTT |
| CT 2nd generation | 5′ To 3′ Primer Sequence |
| F3_C4 | GGTGGGGTAAAGGCCTACCAAG |
| B3_C4 | GCCGGTGCTTCTTTACCTGG |
| FIP_C4 | TGGGCAGTGTCTCAGTCCCATTTTTGCTAT |
| | GACGTCTAGGCGGA |
| BIP_C4 | GACGCCGCGTGTGTGATGATTTTTCAGCGG |
| | GTATTAACCGTCTTC |
| LF_C4 | GTTGGCGGCCAATCTCTCAA |
| LB_C4 | GGCTCTAGGGTTGTAAAGCACTTTCG |
| CT 3rd generation | 5′ To 3′ Primer Sequence |
| F3_CX | TGGGGTAAAGGCCTACCAAG |
| B3_CX | CCGGTGCTTCTTTACCTGG |
| FIP_CX | TGGGCAGTGTCTCAGTCCCAGTTTTTGCTA |
| | TGACGTCTAGGCGGA |
| BIP_CX | GACGCCGCGTGTGTGATGAATTTTTCAGCG |
| | GGTATTAACCGTCTT |
| LF_CX | GTTGGCGGCCAATCTCTCAA |
| LB_CX | GGCTCTAGGGTTGTAAAGCACT |

TABLE 2
LAMP primers for Neisseria gonorrhoeae (NG)

| NG 1st generation | 5′ To 3′ Primer Sequence |
|---|---|
| F3_NG5b | GCCTTCGGGTTGTAAAGGAC |
| B3_NG5b | CCCGGGGATTTCACATCC |
| FIP_NG5b | GCACGTAGTTAGCCGGTGCTTATTTTTCAG |
| | GGAAGAAAAGGCCGTTG |
| BIP_NG5b | AGCAGCCGCGGTAATACGTAGTTTTTGCTT |
| | AAGTAACCGTCTGCGC |
| LF_NG5b | CATCGGCCGCCGATATTGG |
| LB_NG5b | CGTTAATCGGAATTACTGGGCGTAA |
| NG 2nd generation | 5′ To 3′ Primer Sequence |
| F3_N2 | ACACTGGGACTGAGACACGGC |
| B3_N2 | TCGCACCCTACGTATTACCGC |
| FIP_N2 | CTTCTTCAGACACGCGGCATGTTTTTCAGC |
| | AGTGGGGAATTTTGGA |
| BIP_N2 | TCAGGGAAGAAAAGGCCGTTGTTTTTGCTG |
| | GCACGTAGTTAGCCG |
| LF_N2 | CAGGCTTGCGCCCATTG |
| LB_N2 | AATATCGGCGGCCGATGAC |

TABLE 3
recipe for primers stocks (10X primer mix CT and NG stocks)

| 100 uM stock | μL | Final concentration (reaction) | |
|---|---|---|---|
| F3 | 2.5 | 0.25 | Mm |
| B3 | 2.5 | 0.25 | μM |
| FIP | 20 | 2 | μM |
| BIP | 20 | 2 | μM |
| LF | 10 | 1 | μM |
| LB | 10 | 1 | μM |
| Water | 35 | | |

Chlamydia trachomatis (CT) and Neisseria gonorrhoeae (NG) 16S rRNA were amplified using LAMP amplification with the primers reported in Table 1 and Table 2. The results of the target amplification and cross reactivity assay are shown in FIG. 31. Successful amplification for CT (Panel A) and NG (Panel B) was achieved when using the corresponding set of primers. Negative controls (NTC) show no amplification before cycle 45. Cross reactivity between different primers set was determined. Panel C shows experiments performed with CT primers. The CT RNA sample shows positive results before cycle 20, while negative control (NTC) and a NG RNA samples show no amplification before cycle 45. Panel D shows experiments performed with NG primers. The CNG RNA sample shows positive results before cycle 20, while negative control (NTC) and a CT RNA samples show no amplification before cycle 40. Thus the RT-LAMP primers provided in Tables 1 and 2 amplify and detect the respective 16S rRNA with high sensitivity and specificity.

### Example 7: Amplification of Polynucleotides from C. trachomatis Using Lyophilized Bead Reagents

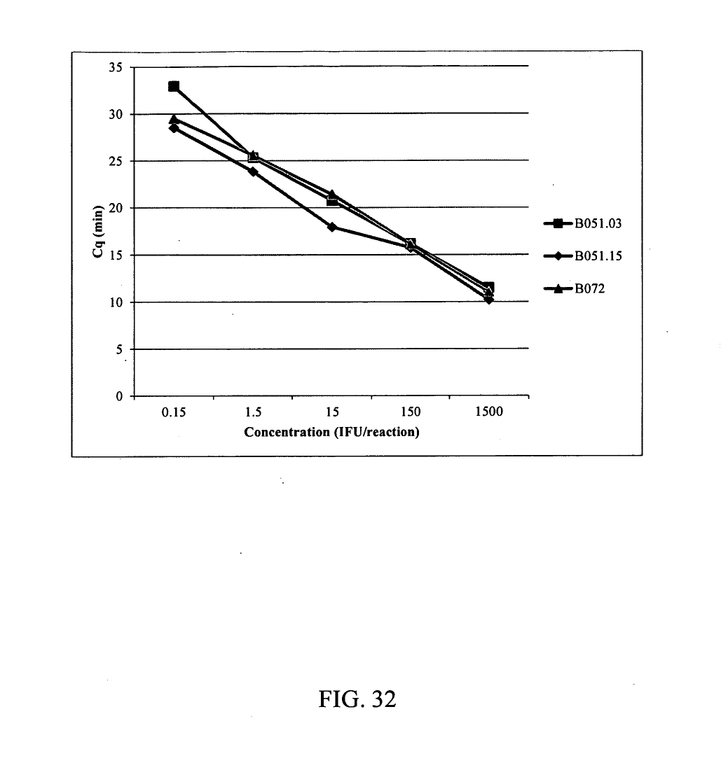

Three different formulations of Lyophilized beads containing LAMP reagents (Tris-HCl pH 8.8, KCl, MgSO4, (NH4)2SO4, Tween20, Bst 2.0 WarmStart® DNA Polymerase, AMV Reverse Transcriptase, Bovine Serum Albumin) were validated for the detection of Chlamydia trachomatis RNA in the amplification module. Nucleic acids from 500 μL of ten-fold diluted samples of Chlamydia trachomatis (ranging from 24 IFU/mL to 240,000 IFU/mL) were extracted with a commercial extraction kit. The purified RNA was mixed with Deoxynucleotide Solution Mix, 10× Chlamydia Trachomatis LAMP primer mixture (20 μM BIP/FIP, 10 μM LB/LF, and 2.5 μM B3/F3), 20× EvaGreen dye and nuclease-free water, and added to the lyophilized beads. After gently mixing, 15 μL of the solution was loaded into 96-wells plate and heated at 63° C. for 50 min and 85° C. for 5 min (heat inactivation) on a LightCycler® 96 Real-Time PCR System. The amplification reactions are shown in FIG. 32. Using the lyophilized beads for LAMP reagents, it is possible to detect as low as 0.15 IFU per amplification reaction (RNA extracted from a 24 IFU/mL sample) within 30 min.

### Example 8: LAMP Amplification in the Amplification Module

Phage lambda DNA was amplified in the multiplex amplification module. A negative and positive LAMP mix solution (Loopamp® RNA amplification kit) was prepared and added to the amplification module. Each solution contained the following:
i. Lambda positive LAMP reaction mix: 50 μL of 2× Reaction Mix (RM) (40 mM Tris-HCl pH 8.8, 20 mM KCl, 16 mM MgSO4, 20 mM (NH4)2SO4, 0.2% Tween20, 1.6 M Betaine and dNTPs 2.8 mM each), 5 μL of Bst 2.0 WarmStart DNA Polymerase (8,000 units/mL, from New England BioLabs), 2.5 μL of Fluorescent Detection Reagent (FD) (Calcein-based solution), 10 μL of BSA (20 mg/mL), 10 μL of 10× lambda LAMP primer mixture (20 μM BIP/FIP, 10 μM LB/LF, and 2.5 μM B3/F3), purified lambda DNA template solution from New England BioLabs, and enough nuclease-free water to bring the total volume of the mix to 100 μL.
ii. Lambda negative LAMP reaction mix: 50 μL of 2× RM, 5 μL of Bst 2.0 WarmStart DNA Polymerase, 2.5 μL of FD, 5 μL of BSA (20 mg/mL), 10 μL of 10× lambda LAMP primer mixture, and enough nuclease-free water to bring the total volume of the mix to 100 μL.

Lambda DNA primers for LAMP are described in “Gansen A, Herrick A M, Dimov I K, Lee L P, Chiu D T. Digital LAMP in a sample self-digitization (SD) chip. Lab Chip. 2012 Jun. 21; 12(12):2247-54,” incorporated herein by reference in its entirety.

Both LAMP mix solutions were loaded onto the amplification module and heated at 63° C. for 50 min in an oven. Fluorescence image was acquired with stereoscope imaging using Leica MZ F1 III stereoscope.

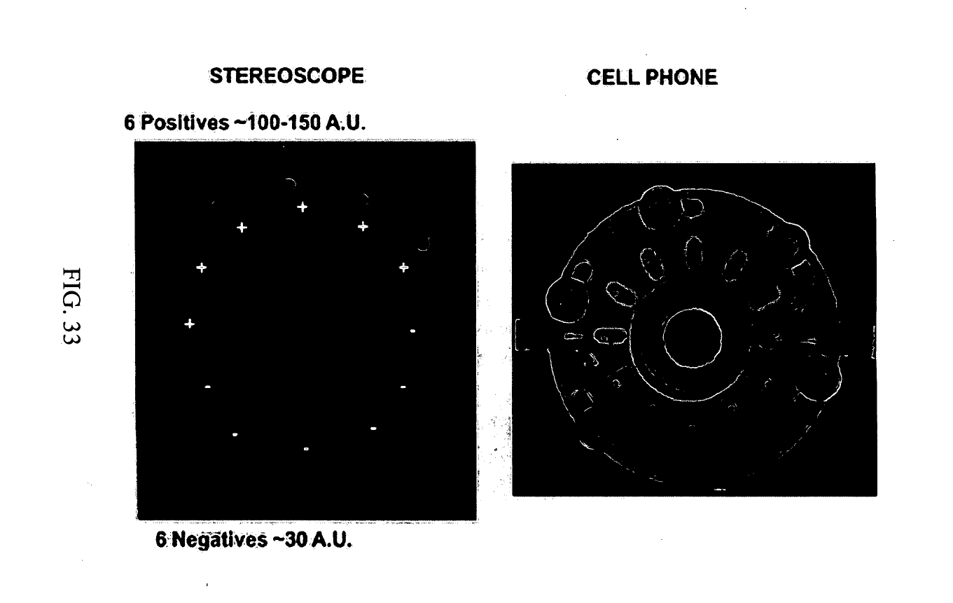

FIG. 33 shows the result of the LAMP amplification in the 3D printed amplification module using lambda DNA as a template. Here, the positive results of the amplification reaction in the amplification module (labeled with a “+”), show a stronger signal than the negative wells (labeled with a “−”).

### Example 9: Multiplex Detection in the Amplification Module

A multiplex amplification device was tested for multiplex detection of Chlamydia trachomatis (CT) or Neisseria gonorrhoeae (NG) 16S ribosomal RNA (rRNA). Specific primers for RT-LAMP amplification of these targets were used, CT_2 for CT and NG_5B for NG (Table 1, Example 6). To amplify CT and NG RNA using RT-LAMP method on the multiplex amplification device, the RT-LAMP mix solutions (Loopamp® RNA amplification kit) contained the following:
i. CT-positive RT-LAMP reaction mix: 50 μL of 2× Reaction Mix (RM) (40 mM Tris-HCl pH 8.8, 20 mM KCl, 16 mM MgSO4, 20 mM (NH4)2SO4, 0.2% Tween20, 1.6 M Betaine and dNTPs 2.8 mM each), 5 μL of Enzyme Mix (EM) (mixture of Bst DNA polymerase and AMV reverse transcriptase), 2.5 μL of Fluorescent Detection Reagent (FD) (Calcein-based solution), 10 μL of BSA (10 mg/mL), 10 μL of 10× CT LAMP primer mixture (20 μM BIP/FIP, 10 μM LB/LF, and 2.5 μM B3/F3), 20 μL of purified CT RNA template solution extracted from NATtrol Chlamydia trachomatis External Run Control Medium (LGVII 434 strain, from ZeptoMetrix Corp.), and enough nuclease-free water to bring the total volume of the mix to 100 μL.
ii. CT-negative RT-LAMP reaction mix: 25 μL of 2× RM, 2.5 μL of EM, 1.25 μL of FD, 5 μL of BSA, 5 μL of 10× CT LAMP primer mixture, and enough nuclease-free water to bring the total volume of the mix to 50 μL.
iii. NG-positive RT-LAMP reaction mix: 50 μL of 2× RM, 5 μL of EM, 2.5 μL of FD, 10 μL of BSA, 10 μl, of 10× NG LAMP primer mixture (20 μM BIP/FIP, 10 μM LB/LF, and 2.5 μM B3/F3), 20 μL of purified NG RNA template solution extracted from NATtrol Neisseria gonorrhoeae External Run Control Medium (Z017 strain, from ZeptoMetrix corn), and enough nuclease-free water to bring the total volume of the mix to 100 μL.
iv. CT-negative RT-LAMP reaction mix: 25 μL of 2× RM, 2.5 μL of EM, 1.25 μL of FD, 5 μL of BSA, 5 μL of 10× NG LAMP primer mixture, and enough nuclease-free water to bring the total volume of the mix to 50 μL.

Each RT-LAMP mix solution was loaded onto the multiplex amplification module and heated at 63° C. for 50 min in an oven. Fluorescence images were acquired with stereoscope imaging using Leica MZ F1 III stereoscope. For both NG and CT targets, each well used for amplification contained RNA from an average of ˜7 elementary bodies. For all experiments a bulk control was run in parallel to monitor amplification and guarantee the absence of false positives. CT and NG RNA was extracted by ZR Viral RNA Kit from Zymo Research, following manufacturer's instructions.

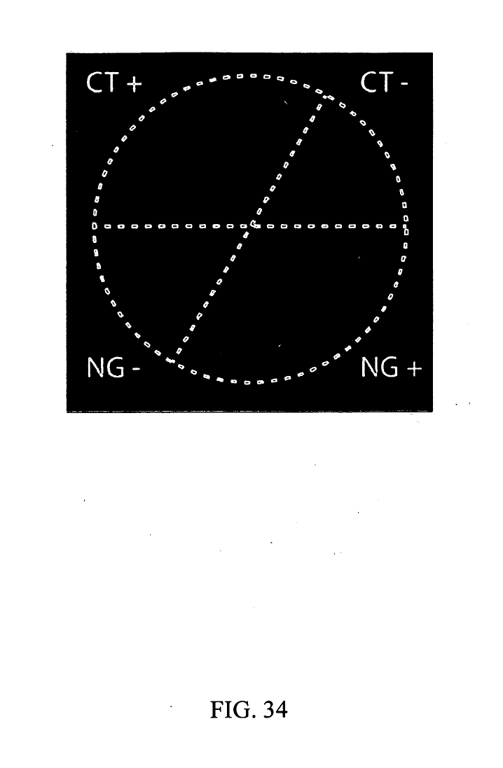

FIG. 34 shows the results of the amplification, providing multiplex detection of Chlamydia trachomatis (CT) and Neisseria gonorrhoeae (NG) 16S RNA in the same device, including negative controls, using LAMP amplification in the 3D printed amplification module.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

1. A method of regulating fluid flow in a device, comprising:
a. providing a device comprising
i. a pressure chamber, said pressure chamber comprising a pressure cap configured to increase or decrease the volume of the pressure chamber;
ii. a plurality of coaxially arranged layers, at least one being a rotor and one being a stator, said coaxially arranged layers having complementary facing surfaces assembled in a frictional, sealed engagement, each layer having at least one passageway with an upstream entry and a downstream outlet capable for successive selective placement in communication to establish plural dedicated flow paths within the assembly; and
iii. a central shaft, said central shaft engaged with the rotor layer, wherein said central shaft comprises threads engaged with the pressure cap to increase or decrease the volume of the pressure chamber, thereby increasing or decreasing pressure in the pressure chamber, and wherein rotation of said central shaft simultaneously rotates the rotor layer while increasing or decreasing pressure in the pressure chamber;
b. loading a sample into a coaxially arranged layer of the device, wherein the rotor layer is in a first position in which all fluidic paths are occluded; and
c. applying torque to the central shaft via a driving module engaged with the central shaft, wherein rotation of the central shaft also moves the pressure cap to reduce the volume of the pressure chamber, thereby increasing pressure in the pressure chamber, said increased pressure slowing the rate of rotation of the rotor layer until said rotor layer reaches a second position, wherein the second position provides an uninterrupted fluidic path, thereby pumping a fluid from a first coaxially arranged layer to a second coaxially arranged layer, and subsequently venting the pressurized pressure chamber, thereby reducing pressure after transfer of said fluid, increasing the rate of rotation of the rotor layer.

2. The method of claim 1, further comprising rotating the rotor layer to a subsequent position by applying torque to the central shaft via a driving module engaged with the central shaft,
a. wherein rotation of the central shaft also moves the pressure cap to reduce the volume of the pressure chamber, thereby increasing pressure in the pressure chamber, said increased pressure slowing the rate of rotation of the rotor layer until said rotor layer reaches the subsequent position, and
b. wherein the subsequent position provides an uninterrupted fluidic path between two coaxially arranged layers, and wherein the fluidic path is linked to the pressure chamber so that the increased pressure in the pressure chamber pumps a fluid along the fluidic path, and subsequently vents the pressurized chamber, thereby reducing pressure after transfer of the fluid, increasing the rate of rotation of the rotor layer.

3. The method of claim 1, wherein said subsequent position is a third, fourth, fifth, sixth, seventh, eighth, ninth, or tenth position.

4. The method of claim 1, wherein said fluid comprises the sample.

5. The method of claim 1, wherein said fluid comprises a reagent.

6. The method of claim 5, wherein said reagent is a lysis reagent, an extraction reagent, a purification reagent, an amplification reagent, or a detection reagent.

7. The method of claim 1, wherein said sample is loaded into said at least one passageway of said coaxially arranged layer.

8. The method of claim 1, wherein said applied torque is constant.

9. The method of claim 1, wherein said driving module comprises a constant torque spring.

10. The method of claim 1, wherein said rotor stops at said second position, restarting after said fluid has transferred from said first coaxially arranged layer to said second coaxially arranged layer.

11. The method of claim 1, wherein the angular velocity of said rotor layer is less than 90%, 80%, 70%, 60%, 50%, 40%, 30%, 20%, 10%, 9%, 8%, 7%, 6%, 5%, 4%, 3%, 2%, or 1% of the initial velocity of the rotor layer at equilibrium pressure when the rotor layer reaches the second position.

12. The method of claim 2, wherein said rotor stops at said subsequent position, restarting after said fluid has transferred from said first coaxially arranged layer to said second coaxially arranged layer.

13. The method of claim 2, wherein the angular velocity of said rotor layer is less than 90%, 80%, 70%, 60%, 50%, 40%, 30%, 20%, 10%, 9%, 8%, 7%, 6%, 5%, 4%, 3%, 2%, or 1% of the initial velocity of the rotor layer at equilibrium pressure when the rotor layer reaches the subsequent position.

14. The method of claim 1, wherein said device comprises a reagent layer comprising at least one reagent solution.

15. The method of claim 14, wherein said reagent layer is the rotor layer.

16. The method of claim 14, wherein said reagent layer is the first layer.

17. The method of claim 14, wherein said reagent layer comprises a holding chamber.

18. The method of claim 14, wherein said reagent layer comprises a surface on the interior of said pressure chamber.

19. The method of claim 14, wherein said reagent layer comprises lysis buffer, a wash buffer, and an elution buffer, each buffer occupying a separate passageway in said reagent layer.

20. The method of claim 1, wherein said rotor layer is the first layer.

21. The method of claim 1, wherein said rotor layer is the second layer.

22. The method of claim 1, wherein said rotor layer comprises a reagent solution.

23. The method of claim 1, wherein the second layer comprises a collection chamber, an analytic chamber, or a detection chamber.

24. The method of claim 1, wherein at least one of said coaxially arranged layers comprises a pre-loaded reagent.

25. The method of claim 1, wherein said pressure chamber comprises a pre-loaded reagent.

26. The method of claim 25, wherein said pre-loaded reagent is lyophilized.

27. An automated device, comprising:
a. a housing having an interior surface;
b. a central shaft comprising a threaded section, wherein the central shaft rotates relative to the housing;
c. a constant force spring engaged with said central shaft, wherein said constant force spring generates torque to rotate said central shaft;
d. a pressure cap comprising:
i. a gasket capable of engaging with the interior surface of the housing to create an airtight seal; and
ii. a central column having threads, wherein the threads engage the threaded section of the central shaft; and
e. a plurality of coaxially arranged layers, at least one being a rotor and one being a stator, said coaxially arranged layers having complementary facing surfaces assembled in a frictional, sealed engagement, each layer having at least one passageway with an upstream entry and a downstream outlet capable for successive selective placement in communication to establish plural dedicated flow paths within the assembly,
wherein rotation of said rotor selectively connects passageways of different coaxial layers, thereby serially forming and disrupting a plurality of fluid paths, wherein a first coaxially arranged layer engages with the interior surface of the housing to create an airtight seal, the surface of the first layer with the housing and the pressure cap thereby forming a compartment,
wherein rotation of the central shaft relative to the housing compresses the compartment, thereby generating pressure against the upstream surface of the first coaxially arranged layer; and
wherein rotation of the central shaft relative to the housing decreases in angular velocity due to increased pressure in said compartment until said rotor aligns with said stator to generate an aligned path for release of said pressure in said compartment.

28. The automated device of claim 1, wherein said aligned path provides a fluidic path for transfer of a fluid from a passageway in the rotor layer through a passageway in the stator layer.

29. The automated device of claim 1, wherein said aligned path further provides a path for air pressure to be released from said compartment, thereby increasing the angular velocity of the rotation of the central shaft.

30. A method of detecting a target nucleic acid in a sample, comprising:
a. providing an integrated device comprising a pressure chamber, an extraction module, an amplification module, and a driving module;
b. adding a sample to said extraction module;
c. initiating said driving module to generate torque to rotate a central shaft, thereby initiating isolation of nucleic acids from said sample and transfer of said nucleic acids to an amplification module, wherein said rotation results in increased pressure in said pressure chamber, thereby increasing resistance to said rotation until a fluid is transferred along a fluidic path created by said rotation, thereby alleviating said pressure through venting of said pressure chamber along the fluidic path; and
d. detecting the presence or absence of a target nucleic acid in said amplification module.

31. The method of claim 30, wherein said integrated device comprises a plurality of coaxially-arranged layers, and wherein friction between said coaxially-arranged layers affects said resistance to said rotation of the central shaft.

32. The method of claim 30, wherein the diver module comprises a constant torque spring motor.

33. The method of claim 30, wherein said detection comprises performing an amplification reaction.

34. The method of claim 33, wherein said amplification reaction is RT-LAMP.

35. The method of claim 30, wherein said amplification module comprises lyophilized reagents.
